(19) United States
(12) Patent Application Publication
(10) Pub. No.: US 2009/0203585 A1
(43) Pub. Date: Aug. 13, 2009

(54) CAPREOMYCIN DERIVATIVES AND THEIR USE AS ANTIBACTERIALS

(76) Inventors: Joseph P. Lyssikatos, Piedmont, CA (US); Steven Mark Wenglowsky, Boulder, CO (US); D. David Hennings, Loveland, CO (US); Daniel John Watson, Lafayette, CO (US)

Correspondence Address: ARRAY BIOPHARMA, INC., 3200 WALNUT STREET, BOULDER, CO, 80301, US

(21) Appl. No.: 12/299,258

(22) PCT Filed: Mar. 13, 2007
(86) PCT No.: PCT/US07/63884

§ 371 Date: Nov. 21, 2008

Related U.S. Application Data
(60) Provisional application No. 60/796,350, filed on May 1, 2006.

(51) Int. Cl. A61K 38/12; C07K 7/06; C07K 1/06; C12P 21/04; A61P 31/04

(52) U.S. Cl. 514/11; 530/321; 530/345; 435/71.1

(57) ABSTRACT

The present invention relates to phenylurea derivatives of capreomycin I, IIB, IIA, or IB, and metabolites and pharmaceutically acceptable salts and solvates thereof. The compounds of the present invention are useful as antibacterial agents for treating bacterial infections and for treating disorders caused by bacterial infections. The present invention also relates to pharmaceutical compositions containing such compounds and to methods of treating bacterial infections by administering such compounds. The present invention also relates to methods of preparing such compounds.

This application claims priority to U.S. Provisional Application Ser. No. 60/796,350, filed 1 May 2006, which is incorporated herein by reference in its entirety.

### FIELD OF THE INVENTION

The present invention provides phenylurea analogs of capreomycin and compositions comprising said analogs, which are useful as antibacterial agents for treating infections caused by Gram-positive or Gram-negative pathogens. The present invention also provides methods of treating bacterial infections.

### BACKGROUND

Bacterial pathogens usually fall in one of two groups: Gram-positive or Gram-negative. Antibacterial agents (including antibiotics) often exhibit selective activity for either Gram-positive or Gram-negative pathogens. Antibacterial agents that target both classes of pathogens are regarded as having broad spectrum activity.

There are many known classes of antibacterial agents such as the penicillins and cephalosporins, tetracyclines, sulfonamides, monobactams, fluoroquinolones and quinolones, glycopeptides, aminoglycosides, polymixins, macrolides, lincosamides, trimethoprim and chloramphenicol. The mechanisms of action of these various classes of antibacterial agents vary.

Resistant strains have evolved/arisen among Gram-positive pathogens such as Staphylococci, Streptococci, Mycobacteria and Enterococci, making the eradication of these strains very difficult. Examples of such strains include methicillin-resistant Staphylococcus aureus (MRSA), methicillin-resistant coagulase negative Staphylococci (MRCNS), penicillin-resistant Streptococcus pneumoniae and multiply-resistant Enterococcus faecium.

Resistance to aminoglycosides, β-lactams (penicillins and cephalosporins) and chloramphenicol analogs is often expressed in pathogenic bacteria. This type of resistance is due to the bacteria-mediated modification of the antibacterial agent through either cleavage of the drug (as in the case with β-lactams) or formation of inactive derivatives (as in the case with aminoglycosides). As for the β-lactams, the resistance observed in clinical isolates is most commonly a result of the expression of “penicillinase” (a β-lactamase) that hydrolytically cleaves the β-lactam ring, thereby inactivating the antibacterial agent.

A more recent threat is the emergence of vancomycin-resistant (VRE) strains of enterococci (Woodford N., 1998, J. Medical Microbiology, 47(10):849-62). VRE strains are frequent causes of hospital-acquired infections and are unfortunately inherently resistant to most antibiotics. Vancomycin inhibits bacterial cell wall synthesis by binding to the terminal D-Ala-D-Ala residues of the cell wall peptidoglycan precursor. The high level vancomycin resistance of VRE isolates is termed VanA and is mediated by genes located on a transposable element which changes the terminal D-Ala-D-Ala residues to D-Ala-D-lac, thereby reducing the affinity for vancomycin.

Capreomycin is a cyclic homopentapeptide obtained from fermentation of Streptomyces caprolus (Herr, E. B., Jr., et al., 1960, Proc. Ind. Acad. Sci., 69:134) and is produced as a four-component mixture, with capreomycin IA and IB present as major products, and IIA and IIB as minor ones. Capreomycin has potent activity against mycobacteria, with little activity against other genera of bacteria.

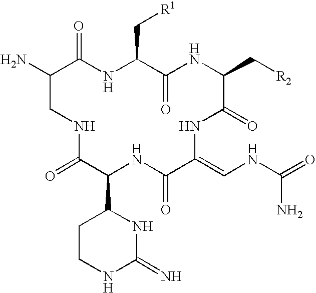

Capreomycin IA: R¹═OH,

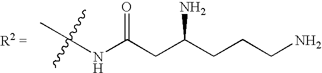

Capreomycin IB: R¹═H,

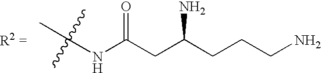

Capreomycin IIA: R¹═OH, R²═NH₂
Capreomycin IIB: R¹═H, R¹═NH₂

Capreomycin itself is used clinically as a second-line treatment for tuberculosis but is not efficacious against most Gram-positive bacteria (as in the case with Staphylococcus) or Gram-negative bacteria (as in the case with Escherichia coli). Certain alkyl-, cycloalkyl- and halogen-substituted phenylurea analogs of capreomycin have been demonstrated to be broad-spectrum (Gram-negative and Gram-positive) antibacterials, especially against resistant strains (Dirlam, et al., Bioorganic and Medicinal Chemistry Letters, 1997, 7(9), 1149-1152).

In light of the rapid emergence of multidrug-resistant bacterial pathogens, the development of antibacterial agents that are effective against both Gram-positive and Gram-negatives pathogens, irrespective of their resistance profiles, and particularly against VRE and MRSA, is urgently needed.

### SUMMARY

The present invention relates to phenylurea analogs of capreomycin and the use thereof in the treatment of microbial infections in a mammal. More specifically, one aspect of the invention provides compounds of the general Formula I

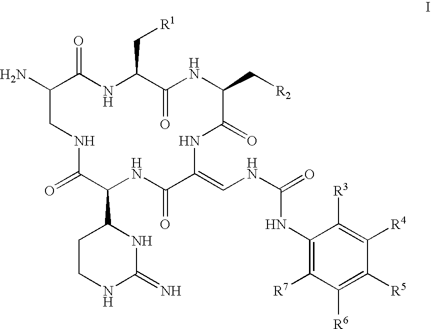

and solvates, metabolites, and pharmaceutically acceptable salts and prodrugs thereof wherein R¹, R², R³, R⁴, R⁵, R⁶ and R⁷ are as defined herein.

Further provided are compounds of Formula IIB

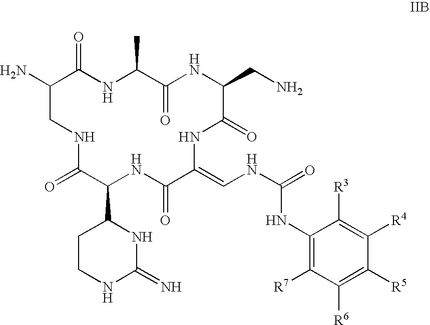

and solvates, metabolites, salts and pharmaceutically acceptable prodrugs thereof, wherein R³, R⁴, R⁵, R¹ and R⁷ are as defined herein.

Further provided are compounds of Formula IA

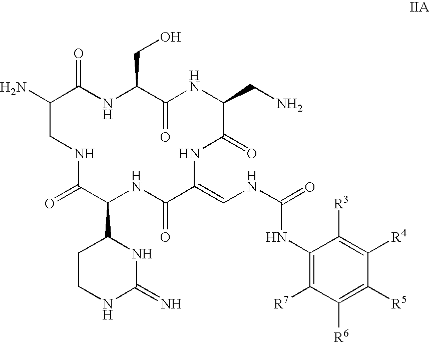

and solvates, metabolites, salts and pharmaceutically acceptable prodrugs thereof, wherein R³, R⁴, R⁵, R¹ and R⁷ are as defined herein.

Further provided are compounds of Formula IB

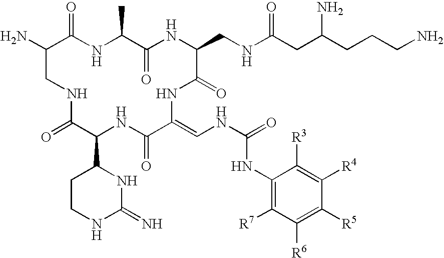

and solvates, metabolites, salts and pharmaceutically acceptable prodrugs thereof, wherein R³, R⁴, R⁵, R⁶ and R⁷ are as defined herein.

Another aspect of the invention provides compositions comprising one or more compounds of Formula I, IIB or IIA. Another aspect of the invention provides compositions comprising one or more compounds of Formula IB.

Another aspect of the invention provides methods of preventing or treating a bacterial infection in a mammal, comprising administering to said mammal in need of such treatment an effective amount of a compound of Formula I, IIB or IIA or a solvate, metabolite, or pharmaceutically acceptable salt or prodrug thereof. Another aspect of the invention provides methods of preventing or treating a bacterial infection in a mammal, comprising administering to said mammal in need of such treatment an effective amount of a compound of Formula IB or a solvate, metabolite, or pharmaceutically acceptable salt or prodrug thereof.

Another aspect of the invention provides methods of preventing or treating disorders related to bacterial infections, comprising administering to a mammal in need of such treatment an effective amount of a compound of Formula I, IIB or IIA or a solvate, metabolite, or pharmaceutically acceptable salt or prodrug thereof. Another aspect of the invention provides methods of preventing or treating disorders related to bacterial infections, comprising administering to a mammal in need of such treatment an effective amount of a compound of Formula IB or a solvate, metabolite, or pharmaceutically acceptable salt or prodrug thereof.

Bacterial infections which may be treated or prevented in mammals according to the methods of the present invention include, but are not limited to, hospital acquired (nosocomial) infections, pneumonia, otitis media, sinusitis, bronchitis, tonsillitis, mastoiditis, pharyngitis, rheumatic fever, glomerulonephritis, respiratory tract infections, blood and tissue infections such as edocarditis and osteomyelitis, uncomplicated skin and soft tissue infections and abscesses, complicated skin and skin structure infections, puerperal fever, uncomplicated acute urinary tract infections, complicated urinary tract infections, urethritis, cervicitis, sexually transmitted diseases, toxin diseases such as food poisoning and toxic shock syndrome, ulcers, systemic febrile syndromes, Lyme disease, conjunctivitis, keratitis, dacrocystitis, gastroenteritis, antibiotic-associated diarrhea, colitis, pseudomembraneous colitis, odontogenic infection, persistent cough, gas gangrene, atherosclerosis and cardiovascular disease.

Additional disorders related to bacterial infections which may be treated or prevented in mammals according to the methods of the present invention include bovine respiratory disease, dairy cow mastitis, swine respiratory disease, swine enteric disease, cow foot-rot, cow hairy warts, cow pink eye, skin and soft tissue infections in dogs and cats, and dental and mouth infections in dogs and cats.

A particular embodiment includes a method of treating infections in mammals caused by infection by C. difficile, comprising administering to said mammal an effective amount of a compound of Formula IIB or a solvate, metabolite, or pharmaceutically acceptable salt or prodrug thereof.

Another aspect of the invention includes methods of preparing compounds of Formula I, IIB or IIA.

Another aspect of the invention includes methods of preparing compounds of Formula IB.

Another aspect of the invention includes kits comprising a compound of Formula I or IIB or a solvate, metabolite, or pharmaceutically acceptable salt or prodrug thereof, and optionally a package insert or label indicating a treatment.

Also provided are compounds of Formula I, IIB or IIA for use in therapy. Also provided are compounds of Formula IB for use in therapy.

An additional aspect of the invention is the use of a compound of Formula I, IIB or IIA in the manufacture of a medicament for treating bacterial infections in a mammal. An additional aspect of the invention is the use of a compound of Formula IB in the manufacture of a medicament for treating bacterial infections in a mammal.

Additional advantages and novel features of the invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### BRIEF DESCRIPTION OF THE FIGURE

The accompanying drawing, which is incorporated herein and forms a part of the specification, illustrates non-limiting embodiments of the present invention, and together with the description, serves to explain the principles of the invention.

In the FIGURE:

FIG. 1 is an HPLC trace of a product mixture containing the Boc-protected compounds IA-1, IB-2, IIA-2 and IIB-2, prepared as described in Example 69.

### DETAILED DESCRIPTION

Provided are compounds that exhibit potent broad spectrum activity against both Gram-positive microbes such as, but not limited to, Staphylococcus aureus, Staphylococcus haemolyticus, Enterococcus faecalis, Enterococcus faecium, and Streptococcus pneumoniae, and Gram-negative microbes such as, but not limited to, Pseudonomas aeruginosa, Klebsiella pneumoniae, Escherichia coli, Haemophilus influenzae, Citrobacter freundii and Enterobacter spp.

The compounds of the invention are especially noteworthy because of their excellent activity against resistant and multi-resistant strains such as MRSA, VISA, vancomycin-resistant Enterococcus faecium, Linezolid-resistant MRSA, Gentamycin-resistant Enterococcus faecalis, PVL positive MRSA, Synercid-resistant Streptococcus pneumoniae, Escherichia coli resistant to both beta-lactams and fluoroquinolones, multi-resistant (beta-lactams, fluoroquinolines, tetracycline, nitrofurantoin) Citrobacter freundii, multi-resistant Enterobacter spp., beta-lactam and aminoglycoside-resistant Klebsiella pneumoniae and multi-resistant (fluoroquinolone, tetracycline, trimethoprim, sulphamethoxazole, augmentin, nitrofurantoin, gentamycin, amikamycin, cefuroxime and impenem) Psuedonomas aeruginosa.

More specifically, one aspect of the invention provides compounds of the general Formula I

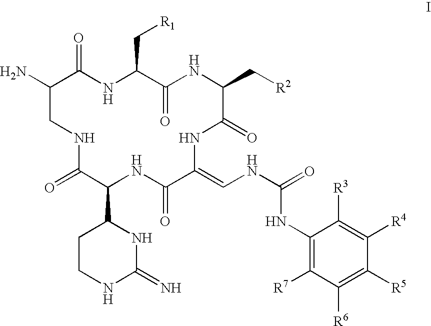

and solvates, metabolites, and pharmaceutically acceptable salts and prodrugs thereof, wherein:

R¹ is OH or H;

R² is NH₂ or;

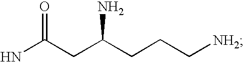

R³, R⁴, R⁵, R⁶ and R⁷ are independently selected from aryl, heteroaryl, X-aryl, X-heteroaryl, hydrogen, halogen, cyano, nitro, trifluoromethyl, difluoromethyl, fluoromethyl, fluoromethoxy, difluoromethoxy, trifluoromethoxy, azido, —OR⁸, SR⁸, —C(O)R⁸, —C(O)OR⁸, NR⁹C(O)OR¹³, —OC(O)R⁸, —NR⁹SO₂R¹³, —SO₂NR⁸R⁹, —NR⁹C(O)R⁸, —C(O)NR⁸R⁹, —NR¹⁰C(O)NR⁸R⁹, —NR¹⁰C(NCN)NR⁸R⁹, —NR⁸R⁹, alkyl, alkenyl, alkynyl, cycloalkyl, cycloalkylalkyl, —S(O)ⱼ(alkyl), —S(O)ⱼ(CR¹¹R¹²)ₘ-aryl, arylalkyl, heteroarylalkyl, heterocyclyl, heterocyclylalkyl, —O(CR¹¹R¹²)-heterocyclyl or —NR⁹(CR¹¹R¹²)ₙ-heterocyclyl,

wherein at least one of R³, R⁴, R⁵, R⁶ and R⁷ is aryl, heteroaryl, X-aryl or X-heteroaryl, and

wherein any of said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from oxo, oxime, halogen, cyano, nitro, fluoromethyl, difluoromethyl, trifluoromethyl, fluoromethoxy, difluoromethoxy, trifluoromethoxy, azido, alkyl, alkenyl, alkynyl, cycloalkyl, cycloalkylalkyl, —OR⁸, —C═NOR⁸, —C(O)R⁸, —C(O)OR⁸, —OC(O)R⁸, —C(O)NR⁸R⁹, —NR⁸R⁹, —NR⁹C(O)OR¹³, —NR⁹C(O)R⁸, —NR¹⁰C(O)NR⁸R⁹, —NR¹⁰C(NCN)NR⁸R⁹, —O(CR¹¹R¹²)ₙ-aryl, —NR⁹(CR¹¹R¹²)ₘ-aryl, —O(CR¹¹R¹²)ₙ-heteroaryl, —NR⁹(CR¹¹R¹²)ₘ-heteroaryl, —O(CR¹¹R¹²)ₙ-heterocyclyl, —NR⁹(CR¹¹R¹²)ₙ-heterocyclyl, —S(O)ⱼ(alkyl), —S(O)ⱼ(CR¹¹R¹²)ₘ-aryl, —SO₂NR⁸R⁹, —NR⁹SO₂R¹³, aryl, heteroaryl, arylalkyl, heteroarylalkyl, heterocyclyl and heterocyclylalkyl;

X is O, O(CR¹¹R¹²)ₙ, NR⁹, (CR¹¹R¹²)ₙ, CR¹¹═CR¹², or S(O)ⱼ(CR¹¹R¹²)ₘ, with the proviso that when R⁵ is CH₂-phenyl, then R³, R⁴, R⁶ and R⁷ are not hydrogen;

R⁸ is hydrogen, trifluoromethyl, alkyl, alkenyl, alkynyl, cycloalkyl, cycloalkylalkyl, aryl, arylalkyl, heteroaryl, heteroarylalkyl, heterocyclyl, heterocyclylalkyl, phosphate, or an amino acid residue, wherein any of said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from oxo, halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl;

R⁹, R¹⁰, R¹¹ and R¹² are independently hydrogen or alkyl, and

R¹³ is trifluoromethyl, alkyl, cycloalkyl, aryl, arylalkyl, heteroaryl, heteroarylalkyl, heterocyclyl, heterocyclylalkyl, wherein any of said alkyl, cycloalkyl, aryl, arylalkyl, heteroaryl, heteroarylalkyl, heterocyclyl and heterocyclylalkyl portions are optionally substituted with one or more groups independently selected from oxo, halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,

or R⁸ and R⁹ together with the atoms to which they are attached form a 4 to 10 membered saturated, partially unsaturated, or fully unsaturated heterocyclic ring, wherein said heterocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR″, aryl, heteroaryl, arylalkyl, heteroarylalkyl, heterocyclyl, and heterocyclylalkyl,

or R⁹ and R¹⁰ together with the atoms to which they are attached form a 4 to 10 membered saturated, partially unsaturated, or fully unsaturated heterocyclic ring, wherein said heterocyclic ring is optionally substituted with one or more groups independently selected from halogen, cyano, nitro, trifluoromethyl, difluoromethoxy, trifluoromethoxy, azido, —NR′SO₂R′″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,

or R⁹ and R¹¹ together with the atoms to which they are attached form a 4 to 10 membered saturated, partially unsaturated, or fully unsaturated heterocyclic ring, wherein said heterocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,

or R⁹ and R¹³ together with the atoms to which they are attached form a 4 to 10 membered saturated, partially unsaturated, or fully unsaturated heterocyclic ring, wherein said heterocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,

or R¹¹ and R¹² together with the atoms to which they are attached form a 4 to 10 membered saturated, partially unsaturated, or fully unsaturated carbocyclic ring, wherein said carbocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR″, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl;

R′, R″ and R′″ independently are hydrogen, alkyl, alkenyl, aryl and arylalkyl, and R″″ is alkyl, alkenyl, aryl and arylalkyl, or any two of R′, R″, R′″ or R″″ together with the atoms to which they are attached form a 4 to 10 membered carbocyclic, aryl, heteroaryl or heterocyclic ring, wherein any of said alkyl, alkenyl, and arylalkyl, and any of said carbocyclic, aryl, heteroaryl and heterocyclic rings or heterocyclic rings are optionally substituted with one or more groups independently selected from halogen, cyano, nitro, trifluoromethyl, difluoromethoxy, trifluoromethoxy, azido, aryl, heteroaryl, arylalkyl, heteroarylalkyl, heterocyclyl, and heterocyclylalkyl;

m is 0, 1, 2, 3, 4 or 5;

n is 1, 2, 3, 4 or 5; and

j is 0, 1 or 2.

In certain embodiments the invention provides compounds of Formula I wherein at least one of R³, R⁴, R⁵, R⁶ and R⁷ is aryl or heteroaryl. Exemplary embodiments include, but are not limited to, phenyl, naphthyl, 2-pyridyl, 3-pyridyl, 4-pyridyl, 2-pyrimidyl, 4-pyrimidyl, 5-pyrimidyl, 1-isoquinolyl, 3-isoquinolyl, 4-isoquinolyl, 2-thienyl, 3-thienyl, 2-benzo[b]thienyl, 3-benzo[b]thienyl, 4-isoxazolyl, and substituted forms thereof. In certain embodiments, R³, R⁴, R⁵, R⁶ and R⁷ are optionally substituted with one or more groups independently selected from halogen, alkyl, CF₃, OCF₃, and heteroalkyl. In certain embodiments, R³, R⁴, R⁶ and R⁷ are optionally substituted with one or more groups independently selected from F, Cl, CF₃, CH₃, OCH₃, and OCF₃.

In certain embodiments the invention provides compounds of Formula I wherein at least one of R³, R⁴, R⁵, R⁶ and R⁷ is X-aryl or X-heteroaryl. Exemplary embodiments include, but are not limited to, O-phenyl, S-phenyl, OCH₂-phenyl, S—CH₂-phenyl, CH₂-phenyl, CH₂CH₂-phenyl, CH═CH-phenyl, CH₂SO₂-phenyl, and NH-phenyl. In certain embodiment, said phenyl is substituted with one or more groups independently selected from halogen, alkyl, CF₃, OCF₃, and heteroalkyl. In certain embodiments, said phenyl is substituted with one or more groups independently selected from F, Cl, CF₃, CH₃, OCH₃, and OCF₃.

In certain embodiments of compounds of Formula I, R¹ is H and R² is β-(S)-lysine amide having the formula

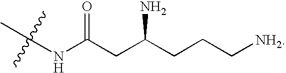

In certain embodiments of compounds of Formula I, R¹ is OH and R² is β-(S)-lysine amide.

Exemplary compounds of Formula I include compounds wherein R¹ is H, R² is β-(S)-lysine amide, R³, R⁵, R⁶ and R⁷ are H, and R⁴ is selected from phenyl, 4-chlorophenyl, 4-trifluoromethylphenyl, 3-trifluoromethylphenyl, 4-tolyl, 4-trifluoromethoxyphenyl, 1-naphthyl, and 2-naphthyl.

Additional exemplary compounds of Formula I include compounds wherein R¹ is H, R² is β-(S)-lysine amide, R³, R⁵, R⁶ and R⁷ are H, and R⁴ is selected from 2-pyridyl, 3-pyridyl, 4-pyridyl, 5-pyrimidyl, 4-isoquinolyl, 2-thienyl, 3-thienyl, 2-chloro-3-thienyl, 3-benzo[b]thienyl, 2-benzo[b]thienyl, 3,5-dimethyl-4-isoxazolyl, and phenoxy.

Additional exemplary compounds of Formula I include compounds wherein R¹ is H, R² is β-(S)-lysine amide, R³, R⁴, R⁶ and R⁷ are H, and R⁵ is selected from phenyl, 4-chlorophenyl, 4-trifluoromethylphenyl, 3,5-ditrifluoromethylphenyl, 1-naphthyl, 2-naphthyl, 2-pyridyl, 3-pyridyl, 4-pyridyl, and 2-thienyl.

Additional exemplary compounds of Formula I include compounds wherein R¹ is H, R² is β-(S)-lysine amide, R³, R⁴, R⁶ and R⁷ are H, and R⁵ is selected from phenoxy, 4-chlorophenyoxy, 4-methylphenoxy, 3,5-difluoromethylphenyl, 4-fluorophenoxy, 4-trifluoromethylphenoxy, and 3-trifluoromethylphenoxy.

Additional exemplary compounds of Formula I include compounds wherein R¹ is H, R² is β-(S)-lysine amide, R³, R⁴, R⁶ and R⁷ are H, and R⁵ is selected from benzenethiolate, 4-methylbenzenethiolate, CH₂═CHPh, CH₂CH₂Ph, CH₂SO₂Ph, NHPh, OCH₂Ph and SCH₂Ph.

Additional exemplary compounds of Formula I include compounds wherein R¹ is H, R² is β-(S)-lysine amide, R⁴, R⁵, R⁶ and R⁷ are H, and R³ is selected from phenyl, 4-chlorophenyl and phenoxy.

The present invention further includes compositions comprising one or more compounds of Formula I. In certain embodiments, the composition comprises compounds of Formula I wherein R³ is aryl, heteroaryl, X-aryl or X-heteroaryl, and R⁴, R⁵, R⁶ and R⁷ are H. In other embodiments, the composition comprises compounds of Formula I wherein R⁴ is aryl, heteroaryl, X-aryl or X-heteroaryl, and R³, R⁵, R⁶ and R⁷ are H. In other embodiments, the composition comprises compounds of Formula I wherein R⁵ is aryl, heteroaryl, X-aryl or X-heteroaryl, and R³, R⁴, R⁶ and R⁷ are H.

In another embodiment, there is provided a compound of Formula IIB

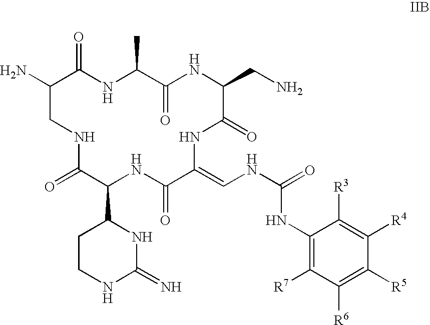

wherein:

R³, R⁴, R⁵, R⁶ and R⁷ are independently selected from aryl, heteroaryl, X-aryl, X-heteroaryl, hydrogen, halogen, cyano, nitro, trifluoromethyl, difluoromethyl, fluoromethyl, fluoromethoxy, difluoromethoxy, trifluoromethoxy, azido, —OR⁸, SR⁸, —C(O)R⁸, —C(O)OR⁸, NR⁹C(O)OR¹³, —OC(O)R⁸, —NR⁹SO₂R¹³, —SO₂NR⁸R⁹, —NR⁹C(O)R⁸, —C(O)NR⁸R⁹, —NR¹⁰C(O)NR⁸R⁹, —NR¹⁰C(NCN)NR⁸R⁹, —NR⁸R⁹, alkyl, alkenyl, alkynyl, cycloalkyl, cycloalkylalkyl, —S(O)ⱼ(alkyl), —S(O)ⱼ(CR¹¹R¹²)ₘ-aryl, arylalkyl, heteroarylalkyl, heterocyclyl, heterocyclylalkyl, —O(CR¹¹R¹²)ₙ-heterocyclyl and —NR⁹(CR¹¹R¹²)-heterocyclyl,

wherein at least one of R³, R⁴, R⁵, R⁶ and R⁷ is aryl, heteroaryl, X-aryl or X-heteroaryl, and

wherein any of said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from oxo, oxime, halogen, cyano, nitro, fluoromethyl, difluoromethyl, trifluoromethyl, fluoromethoxy, difluoromethoxy, trifluoromethoxy, azido, alkyl, alkenyl, alkynyl, cycloalkyl, cycloalkylalkyl, —OR⁸, —C═NOR⁸, —C(O)R⁸, —C(O)OR⁸, —OC(O)R⁸, —C(O)NR⁸R⁹, —NR⁸R⁹, —NR⁹C(O)OR¹³, —NR⁹C(O)R⁸, —NR¹⁰C(O)NR⁸R⁹, —NR¹⁰C(NCN)NR⁸R⁹, —O(CR¹¹R¹²)ₙ-aryl, —NR⁹(CR¹¹R¹²)ₘ-aryl, —O(CR¹¹R¹²)ₙ-heteroaryl, —NR⁹(CR¹¹R¹²)ₘ-heteroaryl, —O(CR¹¹R¹²)ₙ-heterocyclyl, —NR⁹(CR¹¹R¹²)ₙ-heterocyclyl, —S(O)ⱼ(alkyl), —S(O)ⱼ(CR¹¹R¹²)ₘ-aryl, —SO₂NR⁸R⁹, —NR⁹SO₂R³, aryl, heteroaryl, arylalkyl, heteroarylalkyl, heterocyclyl and heterocyclylalkyl;

X is O, O(CR¹¹R¹²)ₙ, NR⁹, (CR¹¹R¹²)ₙ, CR¹¹═CR¹², or S(O)ⱼ(CR¹¹R¹²)ₘ;

R⁸ is hydrogen, trifluoromethyl, alkyl, alkenyl, alkynyl, cycloalkyl, cycloalkylalkyl, aryl, arylalkyl, heteroaryl, heteroarylalkyl, heterocyclyl, heterocyclylalkyl, phosphate, or an amino acid residue, wherein any of said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from oxo, halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R″, —OR′, aryl, heteroaryl, arylalkyl, heteroarylalkyl, heterocyclyl, and heterocyclylalkyl;

R⁹, R¹⁰, R¹¹ and R¹² are independently hydrogen or alkyl, and

R¹³ is trifluoromethyl, alkyl, cycloalkyl, aryl, arylalkyl, heteroaryl, heteroarylalkyl, heterocyclyl, or heterocyclylalkyl, wherein any of said alkyl, cycloalkyl, aryl, arylalkyl, heteroaryl, heteroarylalkyl, heterocyclyl and heterocyclylalkyl portions are optionally substituted with one or more groups independently selected from oxo, halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,

or R⁸ and R⁹ together with the atoms to which they are attached form a 4 to 10 membered saturated, partially unsaturated, or fully unsaturated heterocyclic ring, wherein said heterocyclic ring is optionally substituted with one or more groups independently selected from halogen, cyano, nitro, trifluoromethyl, difluoromethoxy, trifluoromethoxy, azido, —NR′SO₂R″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,

or R⁹ and R¹⁰ together with the atoms to which they are attached form a 4 to 10 membered saturated, partially unsaturated, or fully unsaturated heterocyclic ring, wherein said heterocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,

or R⁹ and R¹¹ together with the atoms to which they are attached form a 4 to 10 membered saturated, partially unsaturated, or fully unsaturated heterocyclic ring, wherein said heterocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,

or R⁹ and R¹³ together with the atoms to which they are attached form a 4 to 10 membered saturated, partially unsaturated, or fully unsaturated heterocyclic ring, wherein said heterocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,

or R¹¹ and R¹² together with the atoms to which they are attached form a 4 to 10 membered saturated, partially unsaturated, or fully unsaturated carbocyclic ring, wherein said carbocyclic ring is optionally substituted with one or more groups independently selected from halogen, cyano, nitro, trifluoromethyl, difluoromethoxy, trifluoromethoxy, azido, —NR′SO₂R″″, —SO₂NR′R″″, —C(O)R′, —C(O)OR″″, —OC(O)R′, —NR′C(O)OR″″, —NR′C(O)R″, —C(O)NR′R″″, —SO₂R″″, NR′R″, NR′C(O)NR″R′″, —NR′C(NCN)NR″R′″, —OR′, aryl, heteroaryl, arylalkyl, heteroarylalkyl, heterocyclyl, and heterocyclylalkyl;

R′, R″ and R′″ independently are hydrogen, alkyl, alkenyl, aryl and arylalkyl, and R″″ is alkyl, alkenyl, aryl and arylalkyl, or any two of R′, R″, R′″ and R″″ together with the atoms to which they are attached form a 4 to 10 membered carbocyclic, aryl, heteroaryl or heterocyclic ring, wherein said alkyl, alkenyl, and arylalkyl, and said carbocyclic, aryl, heteroaryl, and heterocyclic rings are optionally substituted with one or more groups independently selected from halogen, cyano, nitro, trifluoromethyl, difluoromethoxy, trifluoromethoxy, azido, aryl, heteroaryl, arylalkyl, heteroarylalkyl, heterocyclyl, and heterocyclylalkyl;

m is 0, 1, 2, 3, 4 or 5;

n is 1, 2, 3, 4 or 5; and

j is 0, 1 or 2.

In certain embodiments, provided are compounds of Formula IIB wherein:

R³, R⁴, R⁵, R⁶ and R⁷ are independently selected from aryl, heteroaryl, X-aryl, X-heteroaryl, hydrogen, halogen, and alkyl,

wherein at least one of R³, R⁴, R⁵, R⁶ and R⁷ is aryl, heteroaryl, X-aryl or X-heteroaryl, and wherein said aryl, heteroaryl, and alkyl portions are optionally substituted with one or more groups independently selected from halogen, fluoromethyl, difluoromethyl, trifluoromethyl, fluoromethoxy, difluoromethoxy, trifluoromethoxy, alkyl or —OR⁸; and

R⁸ is hydrogen, trifluoromethyl or alkyl.

In certain embodiments, provided are compounds of Formula IIB wherein at least one of R³, R⁴, R⁵, R⁶ and R⁷ is aryl. For example, in certain embodiments, one of R³, R⁴, R⁵, R⁶ and R⁷ is phenyl or naphthyl and the remaining are independently selected from H, halogen, and alkyl. In certain embodiments, said phenyl and naphthyl are optionally substituted with one or more groups independently selected from halogen, alkyl, OR⁸, fluoromethyl, difluoromethyl, trifluoromethyl, fluoromethoxy, difluoromethoxy, and trifluoromethoxy.

In certain embodiments wherein one of R³, R⁴, R⁵, R⁶ and R⁷ is phenyl or naphthyl, the remaining are independently selected from H, F, Cl, methyl, ethyl, propyl, isopropyl, or butyl. Exemplary embodiments include phenyl or naphthyl optionally substituted with one or more groups independently selected from F, Cl, CF₃, CH₃, OCH₃, and OCF₃. Further exemplary embodiments include phenyl, 1-naphthyl, 2-naphthyl, 2-chlorophenyl, 3-chlorophenyl, 4-chlorophenyl, 2-fluorophenyl, 3-fluorophenyl, 4-fluorophenyl, 2-methylphenyl, 3-methylphenyl, 4-methylphenyl, 2-methoxyphenyl, 3-methoxyphenyl, 4-methoxyphenyl, 2-trifluoromethylphenyl, 3-trifluoromethylphenyl, 4-trifluoromethylphenyl, 2-trifluoromethoxyphenyl, 3-trifluoromethoxyphenyl, 4-trifluoromethoxyphenyl, and 4-(3,5-bis-trifluoromethylphenyl).

In another embodiment, there is provided a compound of Formula IB

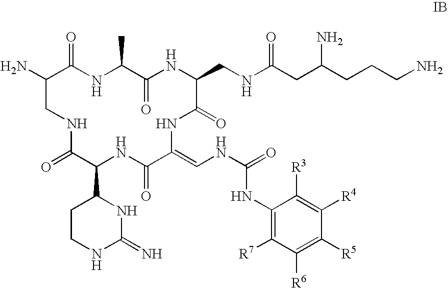

and solvates, metabolites, salts and pharmaceutically acceptable prodrugs thereof, wherein R³, R⁴, R⁵, R⁶ and R⁷ are as defined for Formula IIB.

Particular novel compounds of the invention include any one of the following: compounds of Formula IIB, wherein:

R⁴, R⁵, R⁶ and R⁷ are H and R³ is phenyl;

R⁴, R⁵, R⁶ and R⁷ are H and R³ is 4-chorophenyl;

R³, R⁵, R⁶ and R⁷ are H, and R⁴ is phenyl;

R³, R⁵, R⁶ and R⁷ are H, and R⁴ is 4-chlorophenyl;

R³, R⁵, R⁶ and R⁷ are H, and R⁴ is 4-trifluoromethylphenyl;

R³, R⁵, R⁶ and R⁷ are H, and R⁴ is 3-trifluoromethylphenyl;

R³, R⁵, R⁶ and R⁷ are H, and R⁴ is 4-tolyl;

R³, R⁵, R⁶ and R⁷ are H, and R⁴ is 4-trifluoromethoxyphenyl;

R³, R⁵, R⁶ and R⁷ are H, and R⁴ is 1-naphthyl;

R³, R⁵, R⁶ and R⁷ are H, and R⁴ is 2-naphthyl;

R³, R⁴, R⁶ and R⁷ are H, and R⁵ is phenyl;

R³, R⁴, R⁶ and R⁷ are H, and R⁵ is 4-chlorophenyl;

R³, R⁴, R⁶ and R⁷ are H, and R⁵ is 4-trifluoromethylphenyl; or

R³, R⁴, R⁶ and R⁷ are H, and R⁵ is 3,5-bis-trifluoromethylphenyl.

In certain embodiments, provided are compounds of Formula IIB wherein at least one of R³, R⁴, R⁵, R⁶ and R⁷ is heteroaryl, wherein said heteroaryl is optionally substituted with one or more groups independently selected from halogen and alkyl. Examples of such halogen and alkyl substituents include, but are not limited to, F, Cl, methyl, ethyl, propyl, isopropyl, and butyl.

For example, in certain embodiments, provided are compounds of Formula IIB wherein said heteroaryl is 2-pyridyl, 3-pyridyl, 4-pyridyl, 5-pyrimidyl, 4-isoquinolyl, 2-thienyl, 3-thienyl, 2-chloro-thien-3-yl, 2-benzo[b]thienyl, 3-benzo[b]thienyl, and isoxazol-4-yl, wherein said heteroaryl is optionally substituted with one or more groups independently selected from halogen and methyl. Examples of substituted heteroaryls include 2-chlorothien-4-yl and 3,5-dimethylisoxazol-4-yl.

Particular novel compounds of the invention include any one of the following: compounds of Formula IIB, wherein:

R³, R⁵, R¹ and R⁷ are H, and R⁴ is 2-pyridyl;

R³, R⁵, R⁶ and R⁷ are H, and R⁴ is 3-pyridyl;

R³, R⁵, R⁶ and R⁷ are H, and R⁴ is 4-pyridyl;

R³, R⁵, R⁶ and R⁷ are H, and R⁴ is 5-pyrimidyl;

R³, R⁵, R⁶ and R⁷ are H, and R⁴ is 4-isoquinolyl;

R³, R⁵, R⁶ and R⁷ are H, and R⁴ is 2-thienyl;

R³, R⁵, R⁶ and R⁷ are H, and R⁴ is 2-chlorothien-4-yl;

R³, R⁵, R⁶ and R⁷ are H, and R⁴ is 3-thienyl;

R³, R⁵, R⁶ and R⁷ are H, and R⁴ is 2-chloro-thien-3-yl;

R³, R⁵, R⁶ and R⁷ are H, and R⁴ is 2-benzo[b]thienyl;

R³, R⁵, R⁶ and R⁷ are H, and R⁴ is 3-benzo[b]thienyl;

R³, R⁵, R⁶ and R⁷ are H, and R⁴ is 3,5-dimethylisoxazol-4-yl;

R³, R⁴, R⁶ and R⁷ are H, and R⁵ is 2-pyridyl;

R³, R⁴, R⁶ and R⁷ are H, and R⁵ is 3-pyridyl;

R³, R⁴, R⁶ and R⁷ are H, and R⁵ is 4-pyridyl; or

R³, R⁴, R⁶ and R⁷ are H, and R⁵ is 2-thienyl.

In certain embodiments, provided are compounds of Formula IIB wherein at least one of R³, R⁴, R⁶ and R⁷ is X-aryl, wherein said aryl is optionally substituted with one or more groups independently selected from halogen, alkyl, fluoromethyl, difluoromethyl, trifluoromethyl or OR⁸. In particular embodiments, one of R³, R⁴, R⁵, R⁶ and R⁷ is X-aryl, wherein X-aryl is X-phenyl. In certain embodiments, said phenyl optionally substituted with one or more groups independently selected from fluoro, chloro, methyl, trifluoromethyl, or methoxy.

In certain embodiments, X is selected from O, S, SCH₂, CH₂, CH₂CH₂, CH═CH, OCH₃, CH₂SO₂ or NH.

Exemplary embodiments of X-aryl groups include, but are not limited to, X-phenyl, X-(2-chlorophenyl), X-(3-chlorophenyl), X-(4-chlorophenyl), X-(2-fluorophenyl), X-(3-fluorophenyl), X-(4-fluorophenyl), X-(2-methylphenyl), X-(3-methylphenyl), X-(4-methylphenyl), X-(2-methoxyphenyl), X-(3-methoxyphenyl), X-(4-methoxyphenyl), X-(2-trifluoromethylphenyl), X-(3-trifluoromethylphenyl), X-(4-trifluoromethylphenyl), X-(2-trifluoromethoxyphenyl), X-(3-trifluoromethoxyphenyl), X-(4-trifluoromethoxyphenyl), and X-(4-(3,5-bis-trifluoromethylphenyl)), wherein X is O, S, CH₂, CH₂CH₂, CH═CH, CH₂SO₂, or NH.

Particular novel compounds of the invention include any one of the following: compounds of Formula IIB, wherein:

R⁴, R⁵, R⁶ and R⁷ are H and R³ is —O-phenyl;

R³, R⁵, R⁶ and R⁷ are H and R⁴ is —O-phenyl;

R³, R⁵, R⁶ and R⁷ are H and R⁴ is —CH₂-phenyl;

R³, R⁴, R⁶ and R⁷ are H and R⁵ is —O-phenyl;

R³, R⁴, R⁶ and R⁷ are H and R⁵ is —O-(4-chlorophenyl);

R³, R⁴, R⁶ and R⁷ are H and R⁵ is —O-(4-fluorophenyl);

R³, R⁴, R⁶ and R⁷ are H and R⁵ is —O-(4-methylphenyl);

R³, R⁴, R⁶ and R⁷ are H and R⁵ is —O-(4-methoxyphenyl);

R³, R⁴, R⁵ and R⁷ are H and R⁵ is —O-(4-trifluoromethylphenyl);

R³, R⁴, R⁶ and R⁷ are H and R⁵ is —O-(3-trifluoromethylphenyl);

R³, R⁴, R⁶ and R⁷ are H and R⁵ is 4-(3,5-bis-trifluoromethylphenoxy);

R³, R⁴, R⁵ and R⁷ are H and R⁵ is —OCH₂-phenyl;

R³, R⁴, R⁶ and R⁷ are H and R⁵ is —S-phenyl;

R³, R⁴, R⁶ and R⁷ are H and R⁵ is —CH₂-phenyl;

R³, R⁴, R⁶ and R⁷ are H and R⁵ is —CH₂CH₂-phenyl;

R³, R⁴, R⁶ and R⁷ are H and R⁵ is —CH═CH-phenyl;

R³, R⁴, R⁶ and R⁷ are H and R⁵ is —S—CH₂-phenyl;

R³, R⁴, R⁶ and R⁷ are H and R⁵ is —CH₂SO₂-phenyl;

R³, R⁴, R⁶ and R⁷ are H and R⁵ is —NH-phenyl; or

R³, R⁶ and R⁷ are H, R⁴ is Cl, and R⁵ is O-(4-chlorophenyl).

Particular embodiments of this invention are capreomycin IIB derivatives 73 and 74 having the structures:

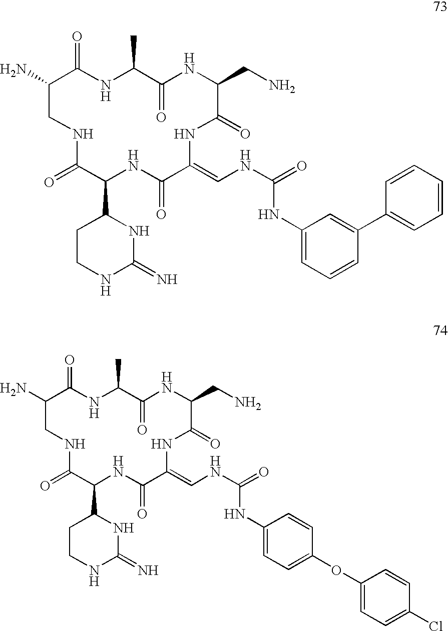

Compounds 73 and 74, which are active against MRSA, were also found to be 4 to 8 fold more active, respectively, against Clostridium difficile than the corresponding capreomycin IB phenyl urea analogs.

Further provided are compounds of Formula IIA

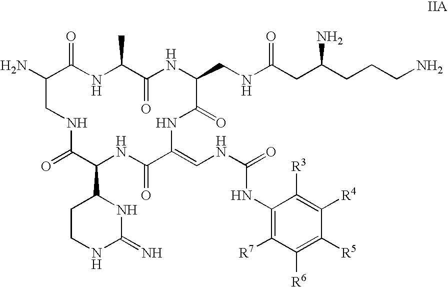

and solvates, metabolites, salts and pharmaceutically acceptable prodrugs thereof, wherein R³, R⁴, R⁵, R⁶ and R⁷ are as defined herein.

In certain embodiments, provided are compounds of Formula IIA wherein:

R³, R⁴, R⁵, R⁶ and R⁷ are independently selected from aryl, heteroaryl, X-aryl, X-heteroaryl, hydrogen, halogen, and alkyl,

wherein at least one of R³, R⁴, R⁵, R⁶ and R⁷ is aryl, heteroaryl, X-aryl or X-heteroaryl, and wherein said aryl, heteroaryl, and alkyl portions are optionally substituted with one or more groups independently selected from halogen, fluoromethyl, difluoromethyl, trifluoromethyl, fluoromethoxy, difluoromethoxy, trifluoromethoxy, alkyl or —OR⁸; and

R⁸ is hydrogen, trifluoromethyl or alkyl.

In certain embodiments, provided are compounds of Formula IIA wherein at least one of R³, R⁴, R⁵, R⁶ and R⁷ is aryl. For example, in certain embodiments, one of R³, R⁴, R⁵, R⁶ and R⁷ is phenyl or naphthyl and the remaining are independently selected from H, halogen, and alkyl. In certain embodiments, said phenyl and naphthyl are optionally substituted with one or more groups independently selected from halogen, alkyl, OR⁸, fluoromethyl, difluoromethyl, trifluoromethyl, fluoromethoxy, difluoromethoxy, and trifluoromethoxy.

In certain embodiments wherein one of R³, R⁴, R⁵, R⁶ and R⁷ is phenyl or naphthyl, the remaining are independently selected from H, F, Cl, methyl, ethyl, propyl, isopropyl, or butyl. Exemplary embodiments include phenyl or naphthyl optionally substituted with one or more groups independently selected from F, Cl, CF₃, CH₃, OCH₃, and OCF₃. Further exemplary embodiments include phenyl, 1-naphthyl, 2-naphthyl, 2-chlorophenyl, 3-chlorophenyl, 4-chlorophenyl, 2-fluorophenyl, 3-fluorophenyl, 4-fluorophenyl, 2-methylphenyl, 3-methylphenyl, 4-methylphenyl, 2-methoxyphenyl, 3-methoxyphenyl, 4-methoxyphenyl, 2-trifluoromethylphenyl, 3-trifluoromethylphenyl, 4-trifluoromethylphenyl, 2-trifluoromethoxyphenyl, 3-trifluoromethoxyphenyl, 4-trifluoromethoxyphenyl, and 4-(3,5-bis-trifluoromethylphenyl).

In certain embodiments, provided are compounds of Formula IIA wherein at least one of R³, R⁴, R⁵, R⁶ and R⁷ is heteroaryl, wherein said heteroaryl is optionally substituted with one or more groups independently selected from halogen and alkyl. Examples of such halogen and alkyl substituents include, but are not limited to, F, Cl, methyl, ethyl, propyl, isopropyl, and butyl.

For example, in certain embodiments, provided are compounds of Formula IIA wherein said heteroaryl is 2-pyridyl, 3-pyridyl, 4-pyridyl, 5-pyrimidyl, 4-isoquinolyl, 2-thienyl, 3-thienyl, 2-chloro-thien-3-yl, 2-benzo[b]thienyl, 3-benzo[b]thienyl, and isoxazol-4-yl, wherein said heteroaryl is optionally substituted with one or more groups independently selected from halogen and methyl. Examples of substituted heteroaryls include 2-chlorothien-4-yl and 3,5-dimethylisoxazol-4-yl.

In certain embodiments, provided are compounds of Formula IIA wherein at least one of R³, R⁴, R⁵, R⁶ and R⁷ is X-aryl, wherein said aryl is optionally substituted with one or more groups independently selected from halogen, alkyl, fluoromethyl, difluoromethyl, trifluoromethyl or OR⁸. In particular embodiments, one of R³, R⁴, R⁵, R⁶ and R⁷ is X-aryl, wherein X-aryl is X-phenyl. In certain embodiments, said phenyl optionally substituted with one or more groups independently selected from fluoro, chloro, methyl, trifluoromethyl, or methoxy. In certain embodiments, X is selected from O, S, SCH₂, CH₂, CH₂CH₂, CH═CH, OCH₃, CH₂SO₂ or NH.

Exemplary embodiments of X-aryl groups include, but are not limited to, X-phenyl, X-(2-chlorophenyl), X-(3-chlorophenyl), X-(4-chlorophenyl), X-(2-fluorophenyl), X-(3-fluorophenyl), X-(4-fluorophenyl), X-(2-methylphenyl), X-(3-methylphenyl), X-(4-methylphenyl), X-(2-methoxyphenyl), X-(3-methoxyphenyl), X-(4-methoxyphenyl), X-(2-trifluoromethylphenyl), X-(3-trifluoromethylphenyl), X-(4-trifluoromethylphenyl), X-(2-trifluoromethoxyphenyl), X-(3-trifluoromethoxyphenyl), X-(4-trifluoromethoxyphenyl), and X-(4-(3,5-bis-trifluoromethylphenyl)), wherein X is O, S, CH₂, CH₂CH₂, CH═CH, CH₂SO₂, or NH.

The term “alkyl” as used herein refers to a saturated linear or branched-chain monovalent hydrocarbon radical of one to twelve carbon atoms, wherein the alkyl radical may be optionally substituted independently with one or more substituents described below. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (1-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂ CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, cyclopropyl, cyclobutyl, cyclopentyl, cyclohexyl, cycloheptyl, and cyclooctyl.

The term “alkenyl” refers to linear or branched-chain monovalent hydrocarbon radical of two to twelve carbon atoms with at least one site of unsaturation, i.e., a carbon-carbon, sp² double bond, wherein the alkenyl radical may be optionally substituted independently with one or more substituents described herein, and includes radicals having “cis” and “trans” orientations, or alternatively, “E” and “Z” orientations. Examples include, but are not limited to, ethylenyl or vinyl (—CH═CH₂), allyl (—CH₂CH═CH₂), and 5-hexenyl.

The term “alkynyl” refers to a linear or branched monovalent hydrocarbon radical of two to twelve carbon atoms with at least one site of unsaturation, i.e., a carbon-carbon, sp triple bond, wherein the alkynyl radical may be optionally substituted independently with one or more substituents described herein. Examples include, but are not limited to, ethynyl (—C≡CH) and propynyl (propargyl, —CH₂C≡CH).

The term “alkyl” refers to a radical having the formula RC═CHCHR, wherein R is alkyl, alkenyl, alkynyl, cycloalkyl, heterocycloalkyl, aryl, or heteroaryl, wherein the allyl may be optionally substituted independently with one or more substituents described herein.

The terms “cycloalkyl,” “carbocyclyl,” and “carbocycle” are used interchangeably and refer to a monovalent non-aromatic, saturated or partially unsaturated cyclic hydrocarbon radical having from three to ten carbon atoms. Examples of monocyclic carbocyclic radicals include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl. The cycloalkyl may be optionally substituted independently in one or more substitutable positions with various groups. The term “cycloalkyl” also includes polycyclic (e.g., bicyclic and tricyclic) cycloalkyl structures, wherein the polycyclic structures optionally include a saturated or partially unsaturated cycloalkyl fused to a saturated or partially unsaturated cycloalkyl or heterocycloalkyl ring or an aryl or heteroaryl ring. Bicyclic carbocycles having 7 to 12 atoms can be arranged, for example, as a bicyclo[4,5], [5,5], [5,6] or [6,6] system, and bicyclic carbocycles having 9 or 10 ring atoms can be arranged as a bicyclo [5,6] or [6,6] system, or as bridged systems such as bicyclo[2.2.1]heptane, bicyclo[2.2.2]octane and bicyclo[3.2.2]nonane.

The term “heteroalkyl” refers to saturated linear or branched-chain monovalent hydrocarbon radical of one to twelve carbon atoms, wherein at least one of the carbon atoms is replaced with a heteroatom selected from N, O, or S, and wherein the radical may be a carbon radical or heteroatom radical (i.e., the heteroatom may appear in the middle or at the end of the radical). The heteroalkyl radical may be optionally substituted independently with one or more substituents described herein. The term “heteroalkyl” encompasses alkoxy and heteroalkoxy radicals.

The terms “heterocycloalkyl,” “heterocycle” and “heterocyclyl” refer to a saturated or partially unsaturated carbocyclic radical of 3 to 8 ring atoms in which at least one ring atom is a heteroatom selected from nitrogen, oxygen and sulfur, the remaining ring atoms being C, where one or more ring atoms may be optionally substituted independently with one or more substituent described below. The radical may be a carbon radical or heteroatom radical. The term further includes fused ring systems that include a heterocycle fused to an aromatic group. “Heterocycloalkyl” also includes radicals where heterocycle radicals are fused with aromatic or heteroaromatic rings. Examples of heterocycloalkyl rings include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl and quinolizinyl. Spiro moieties are also included within the scope of this definition. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). An example of a heterocyclic group wherein 2 ring carbon atoms are substituted with oxo (═O) moieties is 1,1-dioxo-thiomorpholinyl. The heterocycle groups herein are unsubstituted or, as specified, substituted in one or more substitutable positions with various groups.

“Aryl” means a monovalent aromatic hydrocarbon radical of 6-20 carbon atoms derived by the removal of one hydrogen atom from a single carbon atom of a parent aromatic ring system. Some aryl groups are represented in the exemplary structures as “Ar”. Aryl includes bicyclic radicals comprising an aromatic ring with a fused non-aromatic ring, a partially unsaturated ring, or an aromatic ring. Typical aryl groups include, but are not limited to, radicals derived from benzene, substituted benzenes, naphthalene, anthracene, biphenyl, indenyl, indanyl, 1,2-dihydronapthalene, 1,2,3,4-tetrahydronapthyl, and the like. The aryl groups herein are unsubstituted or, as specified, substituted in one or more substitutable positions with various groups.

“Heteroaryl”, “heterocyclyl”, and “heterocycle” all refer to a ring system in which one or more ring atoms are a heteroatom, e.g., nitrogen, oxygen, and sulfur. The heterocyclyl radical comprises 1 to 20 carbon atoms and 1 to 6 heteroatoms selected from N, O, P, and S. The heterocyclyl radical may be saturated, partially unsaturated or fully unsaturated. The heterocyclyl radical may be aromatic or non-aromatic. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo[4,5], [5,5], [5,6], or [6,6] system. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heterocyclyl radicals include, but are not limited to, pyridyl, dihydroypyridyl, 4-dialkylaminopyridinium, tetrahydropyridyl (piperidyl), thiazolyl, tetrahydrothiophenyl, sulfur-oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, 3-oxo-tetrahydrofuranyl, 3-oximinio-tetrahydrofuranyl, bis-tetrahydrofuranyl, tetrahydropyranyl, 4-oxo-tetrahydropyranyl, 4-oximino-tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4H-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

The term “halogen” represents fluorine, bromine, chlorine, and iodine.

The term “arylalkyl” means an alkyl moiety (as defined above) substituted with one or more aryl moiety (also as defined above). More preferred arylalkyl radicals are aryl-C₁₋₃-alkyls. Examples include benzyl, phenylethyl, and the like.

The term “heteroarylalkyl” means an alkyl moiety (as defined above) substituted with a heteroaryl moiety (also as defined above). More preferred heteroarylalkyl radicals are 5- or 6-membered heteroaryl-C₁₋₃-alkyls. Examples include oxazolylmethyl, pyridylethyl and the like.

The term “heterocyclylalkyl” means an alkyl moiety (as defined above) substituted with a heterocyclyl moiety (also defined above). More preferred heterocyclylalkyl radicals are 5- or 6-membered heterocyclyl-C₁₋₃-alkyls. Examples include tetrahydropyranylmethyl.

The term “cycloalkylalkyl” means an alkyl moiety (as defined above) substituted with a cycloalkyl moiety (also defined above). More preferred heterocyclyl radicals are 5- or 6-membered cycloalkyl-C₁₋₃-alkyls. Examples include cyclopropylmethyl.

“Substituted alkyl” refers to an alkyl in which one or more hydrogen atoms are each independently replaced with a substituent. Typical substituents include, but are not limited to, F, Cl, Br, I, CN, CF₃, OR, R, ═O, ═S, ═NR, ═N⁺(O)(R), ═N(OR), ═N⁺(O)(OR), ═N—NRR′, —C(═O)R, —C(═O)OR, —C(═O)NRR′, —NRR′, —N⁺RR′R″, —N(R)C(═O)R′, —N(R)C(═O)OR′, —N(R)C(═O)NR′R″, —SR, —OC(═O)R, —OC(═O)OR, —OC(═O)NRR′, —OS(O)₂(OR), —OP(═O)(OR)₂, —OP(OR)₂, —P(═O)(OR)₂, —P(═O)(OR)NR′R″, —S(O)R, —S(O)₂R, —S(O)₂NR, —S(O)(OR), —S(O)₂(OR), —SC(═O)R, —SC(═O)OR, ═O and —SC(═O)NRR′; wherein each R, R′ and R″ is independently selected from H, C₁-C₁₀ alkyl, C₁-C₁₀ alkenyl, C₁-C₁₀ alkynyl, C₆-C₂₀ aryl and C₂-C₂₀ heterocycle. Alkenyl, alkynyl, allyl, cycloalkyl, heteroalkyl, heterocycloalkyl, arylalkyl, heteroarylalkyl, heterocyclylalkyl, cycloalkylalkyl, aryl and heteroaryl groups as described above may also be similarly substituted.

It is to be understood that in instances where two or more radicals are used in succession to define a substituent attached to a structure, the first named radical is considered to be terminal and the last named radical is considered to be attached to the structure in question. Thus, for example, the radical arylalkyl is attached to the structure in question by the alkyl group.

In the compounds of the present invention where a term such as (CR¹¹R¹²)ₘ is used, R¹¹ and R¹² may vary with each iteration of m above 1. For instance, where m is 2, the term (CR¹¹R¹²)ₘ may equal —CH₂CH₂— or —CH(CH₃)C(CH₂CH₃)(CH₂CH₂CH₃)— or any number of similar moieties falling within the scope of the definitions of R¹¹ and R¹².

The compounds of the present invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers, diastereomers mixtures, racemic or otherwise, thereof. Accordingly, the present invention also includes all such isomers, including diastereomeric mixtures, pure diastereomers and pure enantiomers of the compounds of Formula I. The term “enantiomer” refers to two stereoisomers of a compound which are non-superimposable mirror images of one another. The term “diastereomer” refers to a stereoisomer with two or more centers of chirality and whose molecules are not mirror images of one another. Diastereomers have different physical properties, e.g. melting points, boiling points, spectral properties, and reactivities.

Diastereomeric mixtures can be separated into their individual diastereomers on the basis of their physical chemical differences by methods known to those skilled in the art, for example, by chromatography or fractional crystallization. Enantiomers can be separated by converting the enantiomer mixture into a diastereomeric mixture by reaction with an appropriate optically active compound (e.g., alcohol), separating the diastereomers and converting (e.g., hydrolyzing) the individual diastereomers to the corresponding pure enantiomers. Enantiomers can also be separated by use of a chiral HPLC column. Methods for the determination of stereochemistry and the separation of stereoisomers are well known in the art (see discussion in Chapter 4 of “Advanced Organic Chemistry”, 4-th edition, J. March, John Wiley and Sons, New York, 1992).

In the structures shown herein, where the stereochemistry of any particular chiral atom is not specified, then all stereoisomers are contemplated and included as the compounds of the present invention. Where stereochemistry is specified by a solid wedge or dashed line representing a particular configuration, then that stereoisomer is so specified and defined.

A single stereoisomer, e.g., an enantiomer, substantially free of its stereoisomer may be obtained by resolution of the racemic mixture using a method such as formation of diastereomers using optically active resolving agents (Eliel, E. and Wilen, S. Stereochemistry of Organic Compounds, John Wiley & Sons, Inc., New York, 1994; Lochmuller, C. H., (1975) J. Chromatogr., 113(3):283-302). Racemic mixtures of chiral compounds of the present invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Drug Stereochemistry, Analytical Methods and Pharmacology, Irving W. Wainer, Ed., Marcel Dekker, Inc., New York (1993).

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved is reacted with one enantiomer of a chiral compound to form a diastereomeric pair (E. and Wilen, S. “Stereochemistry of Organic Compounds”, John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g. (−)menthyl chloroformate in the presence of base, or Mosher ester, α-methoxy-α-(trifluoromethyl)phenyl acetate (Jacob III, (1982) J. Org. Chem. 47:4165), of the racemic mixture, and analyzing the NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111). By method (3), a racemic mixture of two enantiomers can be separated by chromatography using a chiral stationary phase (Chiral Liquid Chromatography (1989) W. J. Lough, Ed., Chapman and Hall, New York; Okamoto, (1990) J. of Chromatogr. 513:375-378). Enriched or purified enantiomers can be distinguished by methods used to distinguish other chiral molecules with asymmetric carbon atoms, such as optical rotation and circular dichroism.

The compounds of the present invention may comprise geometric isomers. Compounds having a double bound can exist as E or Z isomeric mixtures, which can be separated into their individual E and Z isomers on the basis of their physical chemical differences by methods known to those skilled in the art, for example, by chromatography or fractional crystallization. All such E isomers, Z isomers, and E, Z isomeric mixtures are considered as part of the present invention.

In addition to compounds of Formula I, IIB, IIA and IB, the present invention also includes solvates, pharmaceutically acceptable prodrugs, metabolites, and pharmaceutically acceptable salts of such compounds.

The term “solvate” refers to an aggregate of a molecule with one or more solvent molecules.

A “pharmaceutically acceptable prodrug” is a compound that may be converted under physiological conditions or by solvolysis to the specified compound or to a pharmaceutically acceptable salt of such compound. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the present invention. The amino acid residues include but are not limited to the 20 naturally occurring amino acids commonly designated by three letter symbols and also includes phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, gamma-aminobutyric acid, cirtulline, homocysteine, homoserine, methyl-alanine, para-benzoylphenylalanine, phenylglycine, propargylglycine, sarcosine, methionine sulfone and tert-butylglycine. Particular examples of prodrugs of the present invention include a compound of Formula I covalently joined to a phosphate residue or a valine residue.

Additional types of prodrugs are also encompassed. For instance, free carboxyl groups can be derivatized as amides or alkyl esters. As another example, compounds of the present invention comprising free hydroxy groups may be derivatized as prodrugs by converting the hydroxy group into groups such as, but not limited to, phosphate ester, hemisuccinate, dimethylaminoacetate, or phosphoryloxymethyloxycarbonyl groups, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including, but not limited to, ether, amine and carboxylic acid functionalities, or where the acyl group is an amino acid ester as described above, are also encompassed. Prodrugs of this type are described in J. Med. Chem., 1996, 39, 10. More specific examples include replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonyl-aminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

Free amines can also be derivatized as amides, sulfonamides or phosphonamides. All of these prodrug moieties may incorporate groups including, but not limited to, ether, amine and carboxylic acid functionalities. For example, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, —C(OH)C(O)OY wherein Y is H, (C₁-C₆)alkyl or benzyl, —C(OY₀)Y₁ wherein Y₀ is (C₁-C₄) alkyl and Y₁ is (C₁-C₆)alkyl, carboxy(C₁-C₆)alkyl, amino(C₁-C₄)alkyl or mono-N— or di-N,N—(C₁-C₆)alkylaminoalkyl, —C(Y₂)Y₃ wherein Y₂ is H or methyl and Y₃ is mono-N- or di-N,N—(C₁-C₆)alkylamino, morpholino, piperidin-1-yl or pyrrolidin-1-yl.

For additional examples of prodrug derivatives, see, for example, a) Design of Prodrugs, edited by H. Bundgaard, (Elsevier, 1985) and Methods in Enznymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of prodrugs,” by H. Bundgaard p. 113-191 (1991); c) H. Bundgaard, Advanced Drug Delivery Reviews, 8:1-38 (1992); d) H. Bundgaard, et al., Journal of Pharmaceutical Sciences, 77:285 (1988); and e) N. Kakeya, et al., Chem. Pharm. Bull., 32:692 (1984), each of which is specifically incorporated herein by reference.

A “metabolite” is a pharmacologically active product produced through in vivo metabolism in the body of a specified compound or salt thereof. Such products may result for example from the oxidation, reduction, hydrolysis, amidation, deamidation, esterification, deesterification, enzymatic cleavage, and the like, of the administered compound. Accordingly, the present invention includes metabolites of compounds of Formula I, including compounds produced by a process comprising contacting a compound of the present invention with a mammal for a period of time sufficient to yield a metabolic product thereof.

Metabolites are typically identified by preparing a radiolabelled (e.g., ¹⁴C or ³H) isotope of a compound of the present invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolites, so long as they are not otherwise found in vivo, are useful in diagnostic assays for therapeutic dosing of the compounds of the present invention.

A “pharmaceutically acceptable salt,” unless otherwise indicated, includes salts that retain the biological effectiveness of the free acids and bases of the specified compound and that are not biologically or otherwise undesirable. A compound of the present invention may possess a sufficiently acidic, a sufficiently basic, or both functional groups, and accordingly react with any of a number of inorganic or organic bases, and inorganic and organic acids, to form a pharmaceutically acceptable salt. Examples of pharmaceutically acceptable salts include those salts prepared by reaction of the compounds of the present invention with a mineral or organic acid or an inorganic base, such salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyn-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methanesulfonates, propanesulfonates, naphthalene-1-sulfonates, naphthalene-2-sulfonates, and mandelates. Since a single compound of the present invention may include more than one acidic or basic moiety, the compounds of the present invention may include mono, di or tri-salts in a single compound.

If the inventive compound is a base, the desired pharmaceutically acceptable salt may be prepared by any suitable method available in the art, for example, treatment of the free base with an acidic compound, particularly an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid such as glucuronic acid or galacturonic acid, an alpha hydroxy acid such as citric acid or tartaric acid, an amino acid such as aspartic acid or glutamic acid, an aromatic acid such as benzoic acid or cinnamic acid, a sulfonic acid such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base. Examples of suitable inorganic salts include those formed with alkali and alkaline earth metals such as lithium, sodium, potassium, barium and calcium. Examples of suitable organic base salts include, for example, ammonium, dibenzylammonium, benzylammonium, 2-hydroxyethylammonium, bis(2-hydroxyethyl)ammonium, phenylethyl-benzylamine, dibenzylethylenediamine, and the like salts. Other salts of acidic moieties may include, for example, those salts formed with procaine, quinine and N-methylglucosamine, plus salts formed with basic amino acids such as glycine, ornithine, histidine, phenylglycine, lysine and arginine.

The compounds of Formulas I, IIB and IIA also include other salts of such compounds which are not necessarily pharmaceutically acceptable salts, and which may be useful as intermediates for preparing and/or purifying compounds of I, IIB and IIA and/or for separating enantiomers of compounds of Formulas I, IIB and IIA.

The present invention also includes isotopically-labeled compounds of the present invention which are identical to those recited herein, but for the fact that one or more atoms are replaced by an atom having an atomic mass or mass number different from the atomic mass or mass number usually found in nature. All isotopes of any particular atom or element as specified is contemplated within the scope of the compounds of the present invention, and their uses. Exemplary isotopes that can be incorporated into compounds of the present invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Certain isotopically-labeled compounds of the present invention (e.g., those labeled with ³H and ¹⁴C) are useful in compound and/or substrate tissue distribution assays. Tritiated (i.e., 3H) and carbon-14 (i.e., ¹⁴C) isotopes are useful for their ease of preparation and detectability. Further, substitution with heavier isotopes such as deuterium (i.e., 2H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

The inventive compounds may be prepared using the reaction routes and synthetic schemes as described below, employing the techniques available in the art using starting materials that are readily available or can be synthesized using methods known in the art.

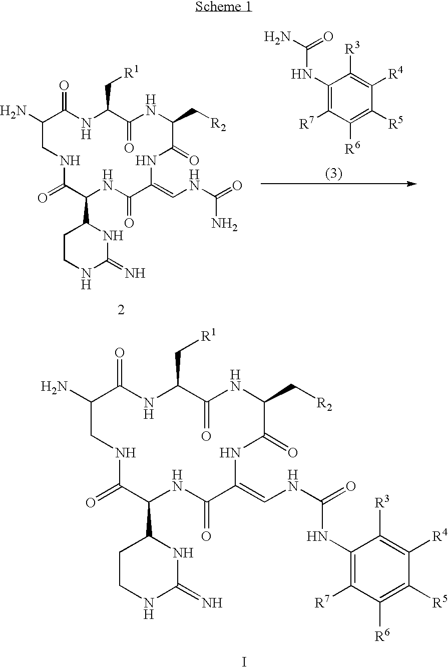

Scheme 1 shows a method of preparing capreomycin derivatives of Formula I. In one embodiment, compounds of Formula I can be prepared by reacting an appropriately substituted phenylurea 3 with capreomycin 2 in water or an organic solvent such as TFA, DMF, acetonitrile, dioxane or DMSO, or a mixed aqueous/organic solvent system such as 50% dioxane and 50% water, in the presence of an inorganic acid such as HCl or H₂SO₄, or an organic acid such as TFA, at a temperature between 0° C. to 150° C., in certain embodiments between 65° C. and 85° C., for a period of 5 minutes to 3 days, in certain embodiments for 1-12 hours.

Commercial capreomycin 2 is a mixture of 4 components wherein R¹ is OH or H and R² is NH₂ or β-(S)-lysine amide. The major components are those wherein R¹ is OH and R² is β-(S)-lysine amide (capreomycin IA), and wherein R¹ is H and R² is β-(S)-lysine amide (capreomycin IB). The minor components are those wherein R¹ is OH and R² is NH₂ (capreomycin IIA) and wherein R¹ is H and R² is NH₂ (capreomycin IIB). When using commercial capreomycin in the reaction shown in Scheme 1, the resultant product is a mixture of four urea derivatives of Formula I, wherein R¹ is OH or H and R² is NH₂ or β-(S)-lysine amide. Accordingly, one aspect of the present invention provides a composition comprising one or more compounds of Formula I.

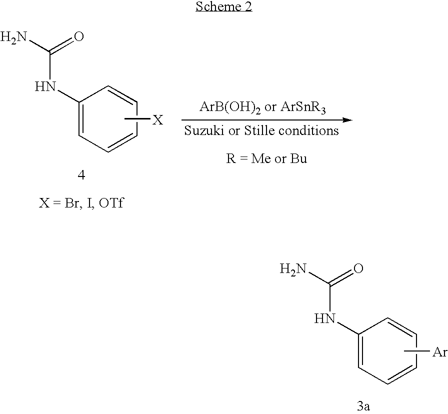

Scheme 2 shows a method of preparing a biaryl urea 3a for use in the method of Scheme 1. In one embodiment, compound 3a can be prepared by reacting a halogen-containing phenylurea 4 (X═Br or I) or an O-trilfate-containing phenylurea 4 under Suzuki or Stille-type palladium-catalyzed conditions with an appropriately substituted aryl or heteroaryl boronic acid ArB(OH)₂ or an appropriately substituted aryl or heteroaryl tin species, for example ArSnMe₃ or ArSnBu₃. Suzuki coupling reactions can be carried out as described by N. Miyaura and A. Suzuki, Chemical Reviews 95, 2457, 1995. Stille reactions are performed using conditions described by V. Farina et al., Org. Reactions, 50, 1, 1997.

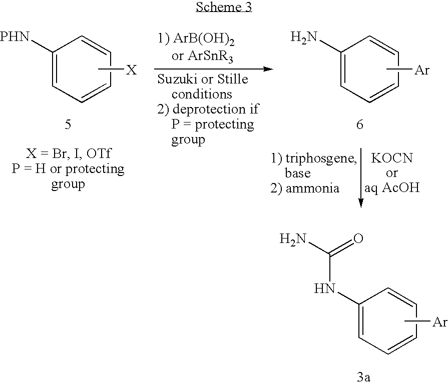

Scheme 3 shows an alternative method of preparing a biaryl urea 3a suitable for use in the method of Scheme 1. In one embodiment, compound 6 can be prepared by reacting a halogen-containing aniline 5 (X═Br or I) or O-trilfate-containing phenylaniline 5, wherein the aniline can be protected or unprotected, under Suzuki or Stille-type palladium-catalyzed conditions with an appropriately substituted aryl or heteroaryl boronic acid ArB(OH)₂ or an appropriately substituted aryl or heteroaryl tin species, for example ArSnMe₃ or ArSnBu₃. In embodiments wherein the aniline moiety of compound 6 is protected, the aniline is deprotected prior to the conversion into compound 3a. Protection and deprotection of anilines are well known to those skilled in the art, and such methods are also described in T. W. Greene and P. G. M. Wuts in “Protective Groups in Organic Synthesis”, 3^{rd} edition, 1999. Biaryl urea 3a can be prepared by reacting substituted aniline 6 or the acid salt of the aniline with either sodium or potassium cyanate in water or an organic solvent such as acetic acid THF, DMF, acetonitrile, dioxane or DMSO, or a mixed solvent system such as 50% acetic acid and 50% DMF, at a temperature between 0° C. to 150° C., in certain embodiments between 5° C. and 35° C., for a period of 5 minutes to 3 days, in certain embodiments for 1-4 hours. In another embodiment, biaryl urea 3a can be prepared as shown in Scheme 3 by reacting substituted aniline 6 with a reagent such phosgene, triphosgene, phenyl chloroformate or 4-nitrophenylchloroformate in an organic solvent such as dichloromethane, dichloroethane, or THF, in the presence of an organic base such as triethylamine, diethylisopropylamine, pyridine, DBU, or 2,6-lutidine, at a temperature between −78° C. to 80° C., in certain embodiments between −78° C. and 35° C., for a period of 5 minutes to 3 days, in certain embodiments for 1-12 hours. The resulting mixture is then treated with an ammonia source such as ammonia gas, ammonium hydroxide, or ammonia dissolved in an organic solvent like methanol or dioxane, at a temperature between −78° C. to 45° C., in certain embodiments between 0° C. and 35° C., for a period of 5 minutes to 3 days, in certain embodiments 1-12 hours.

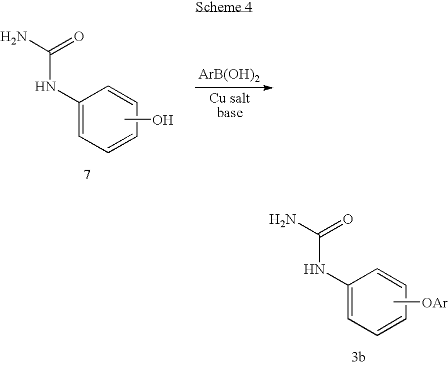

Scheme 4 shows how to prepare a biaryl ether urea 3b suitable for use in the method of Scheme 1. In one embodiment, compound 3b can be prepared by reacting a phenolic urea 7 under copper catalyzed conditions with an appropriately substituted aryl or heteroaryl boronic acid ArB(OH)₂. These copper-catalyzed coupling reactions can be carried out as described by Evans, D, et. al., Tetrahedron Lett. 39, 2937, 1998 or Marcoux, J.-F., et. al., J. Am. Chem. Soc. 119, 10539, 1997.

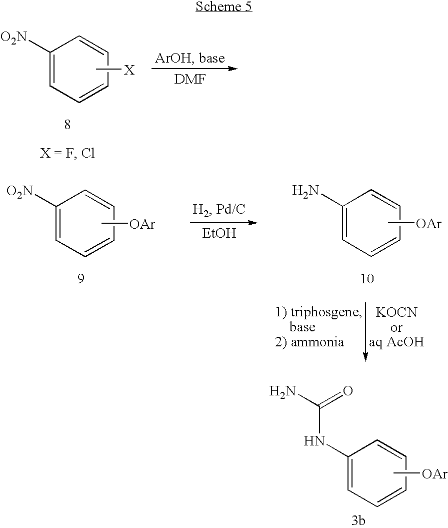

Scheme 5 shows an alternative way to prepare biaryl ether urea 3b suitable for use in the method of Scheme 1. An appropriately substituted aniline 10 can be prepared as shown in Scheme 5 in two steps from compound 8, which can then be transformed into compound 3b.

In one embodiment, compound 9 can be prepared from a halogen-containing (X═F or Cl) nitroaryl compound 8, upon reaction with an appropriately substituted phenoxide in the presence of a base such as NaH, K₂CO₃, or Cs₂CO₃, in an organic solvent such as DMF or DMSO, at a temperature between 0° C. to 150° C. Compound 10 can be prepared from compound 9 by reduction with hydrogen gas and a catalyst such as Pd/C, Raney Ni, PtO₂, or Ru, in a solvent such as EtOH or MeOH. Additional methods of preparing compound 10 from compound 9 include (i) transfer hydrogenation using a hydrogen source such as cyclohexene or formic acid and a catalyst such as Pd/C, (ii) reduction with NaBH₄ and a catalyst such as Pd/C, TiCl₄, NiCl₂₆H₂O or Cu(OAc)₂, or (iii) reduction Zn with HCl, NaOH, or NH₃. Biaryl ether urea 3b can be prepared as shown in Scheme 5 by the methods employed for preparing biaryl ether ureas as described for Scheme 3.

Alternatively, any one of the four capreomycin components IA, IB, IIA or IIB can be prepared according to procedures known to those skilled in the art, such as by fermentation of Streptomyces capreolus A250 (M. S. Brown, et al., 1997, J. Antibiotics, 50(8), 696-7; Wang, M. and S. J. Gould, 1993, J. Org. Chem., 58:5176-5180; S. J. Gould and D. A. Minott, 1992, J. Org. Chem., 57:5214-5217). When published protocols are used as a model for mutagenesis, fermentation, and isolation of capreomycin, it is possible to obtain any of the four components (i.e., IA, IB, IIA or IIB) in a highly enriched state. For example, a mutant of Streptomyces capreolus, CAP47-38, consistently produces a 28:1 ratio in favor of capreomycin IA when fermented in F10a production medium.

Accordingly, another aspect of the present invention provides a method of producing urea analogs of capreomycin IA, capreomycin IB, capreomycin IIA or capreomycin IIB as a single component.

### Isolation of Urea Analogs of Capreomycin IIB

In a particular embodiment, this invention provides methods of preparing and isolating urea analogs of capreomycin IIB having the Formula IIB as described above as single components. Methods of isolating the capreomycin IIB starting material used to prepare compounds of Formula IIB are described in Methods A-D.

### Method A

In general, one embodiment, referred to herein as Method A, provides a method of preparing urea analogs of capreomycin IIB having the Formula IIB, said method comprising:

a) providing a mixture of compounds having the formulas IA, IB, IIA and IIB:

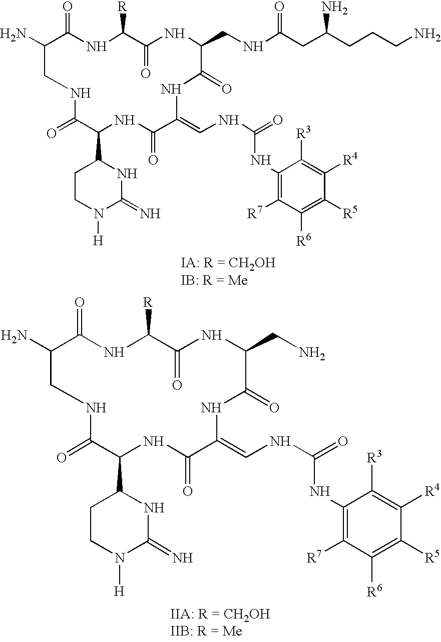

wherein R³, R⁴, R⁵, R⁶ and R⁷ are as defined herein;

b) treating said mixture of compounds IA, IB, IIA and IIB with a reagent that delivers a nitrogen protecting group in the presence of a base and in a suitable organic solvent to provide a mixture of compounds IA-2, IB-2, IIA-2 and IIB-2 having the formulas:

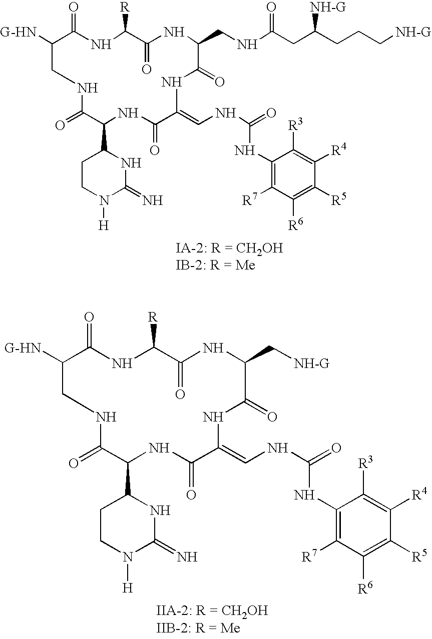

wherein R³, R⁴, R⁵, R⁶ and R⁷ are as defined herein and G is a nitrogen protecting group;

c) treating said mixture of compounds IA-2, IB-2, IIA-2 and IIB-2 with a reagent that delivers an alcohol protecting group in the presence of a base to provide a mixture of compounds IA-3, IB-2, IIA-3 and IIB-2

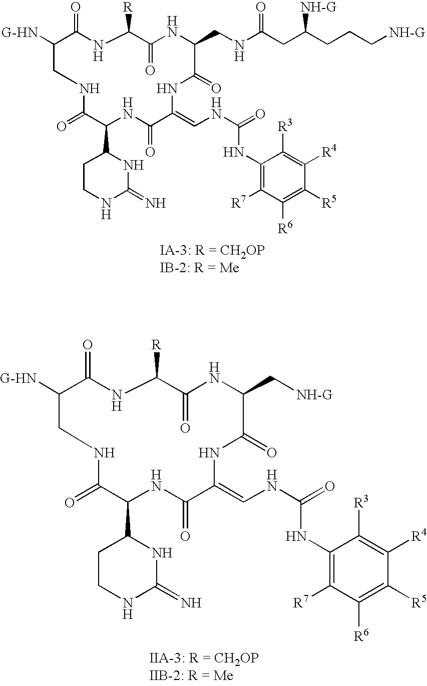

wherein R³, R⁴, R⁵, R⁶ and R⁷ are as defined herein and P is an alcohol protecting group;

d) loading the mixture of compounds IA-3, IB-2, IIA-3 and IIB-2 onto a C18 reverse phase resin;

e) eluting fractions containing compound IIB-2 from said resin with a gradient eluent comprising 15-100% acetonitrile:water;

f) collecting said fractions containing compound IIB-2; and

g) removing said nitrogen protecting group from compound IIB-2 to provide said compound having Formula IIB.

In certain embodiments, Method A further comprises:

f-1) combining said fractions containing compound IIB-2 from step f);

f-2) loading the combined fractions onto a second C18 reverse phase column;

f-3) eluting fractions containing compound IIB-2 from the second column with a gradient eluent comprising 15-100% acetonitrile:water.

Suitable nitrogen protecting groups for use in Method A include, but are not limited to, tert-butylcarbonate (Boc), benzyloxycarbonyl (Cbz), 9-fluorenylmethoxycarbonyl (Fmoc), allyloxycarbonyl (Aloc), 2,9-trimethylsilyl)ethoxycarbonyl (Teoc), and 2,2,2-trichloroethoxycarbonyl (Troc). In one embodiment, the nitrogen protecting group is tert-butylcarbonate (Boc).

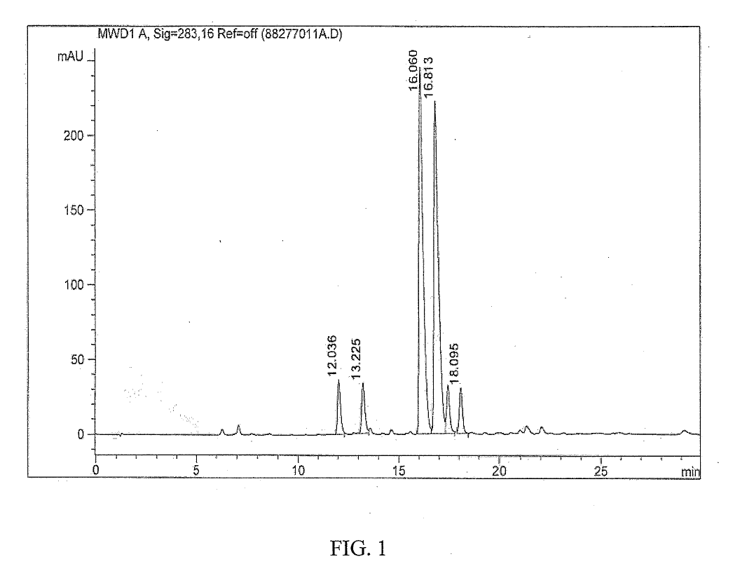

FIG. 1 shows an example of an HPLC trace of a product mixture containing the nitrogen-protected compounds IA-2, IB-2, IIA-2, and IIB-2 (i.e., prior to the alcohol protection step) prepared as described in Example 69, wherein R³, R⁵, R⁶ and R⁷ are H, R⁴ is phenyl, and G is a Boc group. The peak assignments in the HPLC trace of FIG. 1 are as follows:

| | |
|---|---|
| 12.036 minutes: | compound IIA-2 |
| 13.225 minutes: | compound IIB-2 |
| 16.060 minutes: | compound IA-2 |
| 16.813 minutes: | compound IB-2. |

Protection of the alcohol group of compounds IA-2 and IIA-2 to provide the mixture of compounds IA-3, IB-2, IIA-3 and IIB-2 according to Method A provides a means for separating these analogs by medium pressure column chromatography. An HPLC chromatogram (not shown) of the mixture of compounds IA-3, IB-2, IIA-3 and IIB-2 shows that the peaks for compounds IA-3 and IIA-3 are observed about 20-30 minutes later than those for compounds IB-2 and IIB-2. Accordingly, chromatographic separation of the mixture is simplified, since the alcohol-protected compounds IA-3 and IIA-3 are retained on the column resin much longer, and therefore elute much later than compounds IB-2 and IIB-2.

Reagents suitable for use in protecting the alcohol group in Method A include, but are not limited to, silyl chlorides (e.g., tert-butyldiphenylchlorosilane, tertbutyldimethylchlorosilane, chlorotriethylsilane, chlorodiphenylmethylsilane, chlorotriphenylsilane, dimethylthexylsilyl chloride, methylmethoxychlorosilane); dimethylalkylsilanes (e.g., dimethylhexylchlorosilane, dimethyloctylchlorosilane, dimethyloctadecylchlorosilane); trialkylsilanes (e.g., chlorotributylsilane, chlorotrihexylsilane); and acid chlorides (e.g., acetyl chloride, phenylacetyl chloride, benzoyl chloride). In one embodiment, the reagent that delivers an alcohol protecting group is tert-butyldiphenylchlorosilane.

The protection of the alcohol group of the capreomycin IA and IIA analogs can be performed in any suitable organic solvent, such as DMF, and is typically performed at ambient temperature. The reaction is typically performed in the presence of a base. A suitable base for use in the alcohol protection step includes, but is not limited to, imidazole.

In a particular embodiment, the KF (i.e., the moisture content) of the capreomycin IA and IIA analogs is less than 3 wt %, and in a more particular embodiment, less than 1 wt %, prior to the alcohol protection step.

Separation of the mixture of compounds IA-3, IB-2, IIA-3 and IIB-2 by column chromatography is possible over a variety of common absorbents including silica gel, alumina and reverse phase (C18) resin. In one embodiment of Method A, the column absorbent is reverse phase C18 resin. The mixture of compounds IA-3, IB-2, IIA-3 and IIB-2 is loaded onto the column, and the components are eluted with a gradient solvent system comprising, for example 15-100% acetonitrile:water. The eluting fractions can be monitored, for example, using an in-process UV detector/recorder module and can be analyzed further by HPLC to determine the composition of the fraction. The fractions containing the compound IIB-2 are then combined and concentrated and, if desired, can be analyzed for composition and purity.

If compound IIB-2 isolated from the reverse phase chromatography process is contaminated with one or more of the other capreomycin analogs (i.e., IA-3, IB-2 and/or IIA-3), a second purification of compound IIB-2 by column chromatography can be performed as described above.

Removal of the nitrogen-protecting group from compound IIB-2 to provide capreomycin IIB can be performed under standard conditions well know to those skilled in the art. For example, when the nitrogen protecting group is t-butylcarbonate (Boc), the protecting group can be removed by treating the protected compound with aqueous HCl in a suitable solvent such as EtOH/MeOH to provide purified capreomycin IIB. The purified capreomycin IIB can be derivatized as shown in Scheme 1 to provide a compound of Formula IIB.

### Method B

Another embodiment of this invention, referred to herein as Method B, provides a method of preparing compounds of Formula IIB from capreomycin IIB, wherein the capreomycin IIB starting material is obtained via a fermentation process using Saccarothirix mutaiblis subsp. capreolus in the presence of (S)-2-aminoethyl-L-cysteine (AEC). This method is described in detail in Example 74.

AEC is known to be a potent inhibitor of lysine-2,3-aminomutase, the enzyme responsible for converting L-lysine to L-β-lysine (T. P. Chirpich, et al., J. Biol. Chem., 1970, 245:1779-1789). For example, it has been demonstrated that fermentation of Streptomyces griseoverticillatus var. tuberacticus NRRL 3482 in the presence of AEC produces tuberactinamine A, which lacks a β-lysine side chain (B. K, Morse, et al., J. Antibiotics, August 1997, 698-700).

Briefly, Method B comprises inoculating Saccarothirix mutaiblis subsp. capreolus into a seed medium at 29° C. for a suitable period of time, for example, 72 hours. An aliquot of the stage 1 seed is transferred into a flask containing sterile seed medium and is incubated at 29° C. for a suitable period of time, for example, 48 hours. From this second seed stage culture, an aliquot is transferred to a fermentor containing (S)-2-aminoethyl-L-cysteine in a suitable production medium at 29° C., and a peptone is added intermittently as indicated by monitoring of glucose depletion. In one embodiment, the peptone is Soytone Select (Difco). In certain embodiments, the production medium contains L-asparagine and L-alanine. After a suitable fermentation period, the broth (production medium) is recovered and ultrafiltered to recover clean fermentation supernatant. By this method, a product mixture containing capreomycin IIB (76.9% of total capreomycins), capreomycin IA (approximately 7.8% of total capreomycins) and capreomycin IB (approximately 1.2% of total capreomycins) is obtained.

The mixture of capreomycins IIB, IA and IB obtained by this fermentation process can then be reacted with an appropriately substituted phenylurea as described in Scheme 1 to produce a mixture of capreomycin derivatives IIB-1, IA-1 and IB-1. The desired capreomycin derivative of Formula IIB can then be isolated from this mixture by column chromatography as described in Method A.

### Method C

Another embodiment of this invention, referred to herein as Method C, provides a method of preparing compounds of Formula IIB from capreomycin IIB, wherein the capreomycin IIB starting material is obtained via fermentation process using the mutant strain Streptomyces capreolus CAP8-6 according to the method of Brown et al. (J. Antibiotics, August 1997, 696-697). This method is described in detail in Example 75.

Briefly, Method C comprises inoculating agar plugs of Streptomyces capreolus CAP8-6 culture in S-1 medium and incubating at 29° C., 225 rpm for a suitable incubation period, for example 48 hours. The culture is then inoculated into Def-1 medium. L-alanine is optionally added to the culture, and the culture is incubated at 29° C., 225 rpm, for a suitable incubation period, for example 5 days. By this method, a product mixture containing capreomycin IIB and IIA in a ratio of 5:1 with 0.8% supplemental L-alanine or in a ratio of 3:1 without supplemental L-alanine is obtained. The mixture of capreomycin IIB and IIA produced by this method can be reacted with an appropriately substituted phenylurea according to the method of Example 1, Step B to provide a mixture of capreomycin IIB-1 and IIA-1 derivatives. The desired capreomycin derivative of Formula IIB can then be isolated from this mixture by column chromatography as described in Method A.

### Method D

Yet another embodiment of this invention, referred to herein as Method D, provides a method of preparing urea analogs of Formula IIB, comprising reacting a capreomycin derivative having the formula

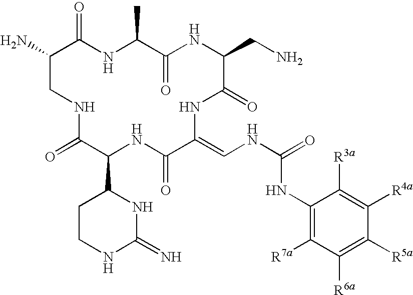

wherein R^{3a}, R^{4a}, R^{5a}, R^{6a} and R^{7a} are as defined for R³, R⁴, R⁵, R⁶ and R⁷, with a substituted phenylurea having the formula

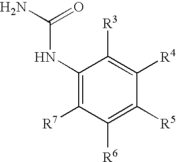

wherein R³, R⁴, R⁵, R⁶ and R⁷ are as defined above, provided that R³ is not the same as R^{3a}, and/or R⁴ is not the same as R^{4a}, and/or R⁵ is not the same as R⁵, and/or R⁶ is not the same as R^{6a}, and/or R⁷ is not the same as R⁷. The reaction can take place in water or an organic solvent such as TFA, DMF, acetonitrile, dioxane or DMSO, or a mixed aqueous/organic solvent system such as 50% dioxane and 50% water, in the presence of an inorganic acid such as HCl or H₂SO₄, or an organic acid such as TFA, at a temperature between 0° C. to 150° C., in certain embodiments between 65° C. and 85° C., for a period of 5 minutes to 3 days, in certain embodiments for 1-12 hours.

### Isolation of Urea Analogs of Capreomycin IIa

In a particular embodiment, this invention provides methods of preparing and isolating urea analogs of capreomycin IIA having the Formula IIA as described above as single components. Methods of isolating the capreomycin IIA starting material used to prepare compounds of Formula IIA are described in Methods E-F

### Method E

Another embodiment of this invention, referred to herein as Method E, provides a method of preparing compounds of Formula IIA from capreomycin IIA, wherein the capreomycin IIA starting material is obtained via fermentation process using the mutant strain Streptomyces capreolus CAP47-38 (Brown et al., J. Antibiotics, August 1997, 696-697) in the presence of the lysine-2,3-aminomutase inhibitor (S)-2-aminoethyl-L-cysteine (AEC).

The mutant strain Streptomyces capreolus CAP47-38 has been demonstrated to produce predominantly capreomycin IA (i.e., a capreomycin analog having a β-lysine side chain) selectively when fermented in F10a production medium. Accordingly, when the CAP47-38 is fermented in the presence of the lysine-2,3-aminomutase inhibitor (S)-2-aminoethyl-L-cysteine (AEC) according to Method E of this invention, the capreomycin IIA can be produced selectively.

Briefly, Method E comprises inoculating agar plugs of Streptomyces capreolus CAP47-38 culture in S-1 medium and incubating at 29° C., 225 rpm for a suitable incubation period, for example 48 hours. The culture is then inoculated into Def-1 medium containing (S)-2-aminoethyl-L-cysteine, and the culture is incubated at 29° C., 225 rpm, for a suitable incubation period, for example 5 days. By this method, a product mixture containing capreomycin IIA is obtained.

The capreomycin IIA obtained by Method E an then be reacted with an appropriately substituted phenylurea as described in Scheme 1 to produce a phenyl urea analog of Formula IIA.

### Method F

Another embodiment of this invention, referred to herein as Method F, provides a method of preparing compounds of Formula IIA from capreomycin IIA, wherein the capreomycin IIA starting material is obtained via fermentation process using the mutant strain Streptomyces capreolus CAP8-6 in a medium supplemented with L-serine according to the method of Brown et al. (J. Antibiotics, August 1997, 696-697).

Briefly, Method F comprises inoculating agar plugs of Streptomyces capreolus CAP8-6 culture in S-1 medium and incubating at 29° C., 225 rpm for a suitable incubation period, for example 48 hours. The culture is then inoculated into Def-1 medium supplemented with 0.8% L-serine and the culture is incubated at 29° C., 225 rpm, for a suitable incubation period, for example 5 days. By this method, a product mixture containing capreomycins IIA and IIB in a ratio of 3:1 is obtained. The mixture of capreomycins IIA and IIB produced by this method can be reacted with an appropriately substituted phenylurea according to the method of Example 1, Step B to provide a mixture of compounds IIA-1 and IIB-1 described above. The desired capreomycin derivative of Formula IIA can then be protected as described in Method A and isolated by reverse phase column chromatography in a manner similar to that described in Method A, with the exception that the fractions containing the compound IIA-3 are isolated.

### Isolation of Urea Analogs of Capreomycin Ib

### Method G

In another embodiment, this invention provides methods of preparing and isolating urea analogs of capreomycin IB having the Formula IB as described above as single components.

In general, one embodiment, referred to herein as Method G, provides a method of preparing urea analogs of capreomycin IB having the Formula IB, said method comprising:

a) providing a mixture of compounds having the formulas IA, IB, IIA and IIB:

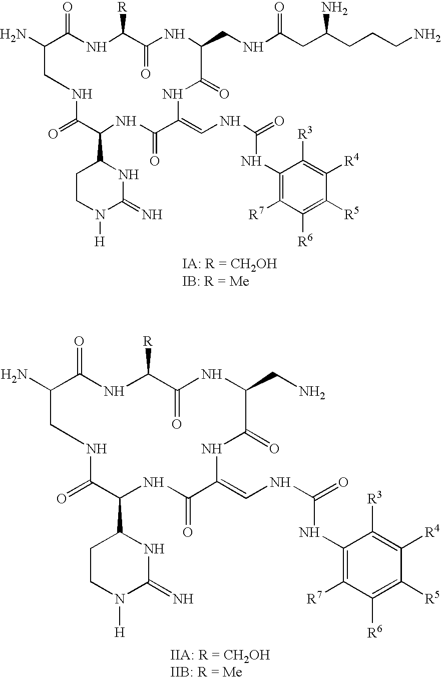

wherein R³, R⁴, R⁵, R⁶ and R⁷ are as defined herein;

b) treating said mixture of compounds IA, IB, IIA and IIB with a reagent that delivers a nitrogen protecting group in the presence of a base and in a suitable organic solvent to provide a mixture of compounds IA-2, IB-2, IIA-2 and IIB-2 having the formulas:

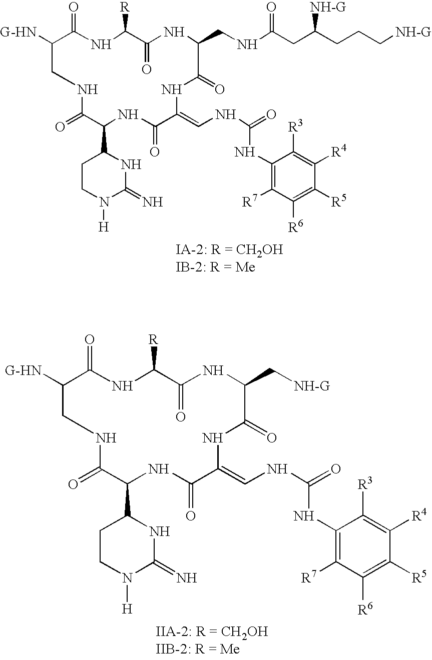

wherein R³, R⁴, R⁵, R⁶ and R⁷ are as defined herein and G is a nitrogen protecting group;

c) treating said mixture of compounds IA-2, IB-2, IIA-2 and IIB-2 with a reagent that delivers an alcohol protecting group in the presence of a base to provide a mixture of compounds IA-3, IB-2, IIA-3 and IIB-2

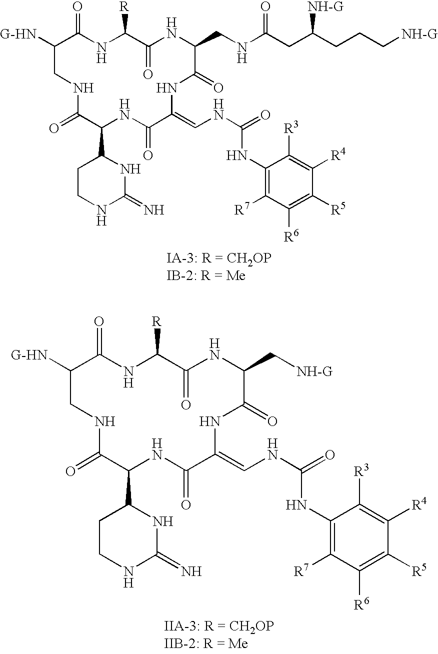

wherein R³, R⁴, R⁵, R⁶ and R⁷ are as defined herein and P is an alcohol protecting group;

d) loading the mixture of compounds IA-3, IB-2, IIA-3 and IIB-2 onto a C18 reverse phase resin;

e) eluting fractions containing compound IB-2 from said resin with a gradient eluent comprising 15-100% acetonitrile:water

f) collecting said fractions containing compound IB-2; and

g) removing the nitrogen protecting groups to provide said compound IB.

In one embodiment, the nitrogen protecting groups are removed by treating compound IB-2 with HCl in an alcoholic solvent such as methanol, ethanol, or a combination thereof. In certain embodiments, the HCl is gaseous HCl. In other embodiments, the HCl is an aqueous solution, for example a 3M solution of HCl in water. The compound IB is then dried to remove residual solvent. In certain embodiments, it was observed that during the drying phase, a portion of compound IB, which is the Z-isomer, was converted to E-isomer (for example, about 5-6 area % of the E isomer was observed):

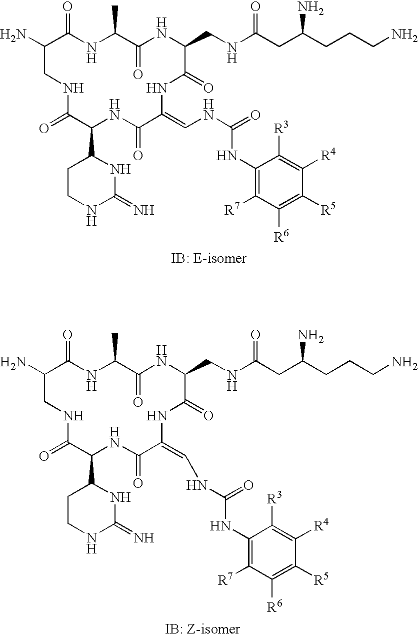

In particular, the level of the E-isomer was found to be related to the levels of residual alcohol and HCl, as well as the temperature during the drying step. It was discovered that the amount of E-isomer that was formed could be reduced to less than 1 area % by drying compound IB under particular conditions. Accordingly, one embodiment of Method G for preparing compound IB further includes:

h) drying compound IB at a temperature of less than or equal to 40° C. until the alcoholic solvent content is less that 10 wt %; and

i) drying compound IB at a temperature greater than or equal to 45° C. until the alcoholic solvent content is less than or equal to 1 wt %.

Referring to step h), convenient temperatures include temperatures in the range of ambient temperature to 40° C. In a particular embodiment, the drying temperature of step h) is about 40° C.

Referring to step i), convenient temperatures include temperatures in the range of 45-100° C., for example about 50° C.

A further aspect of the invention includes methods of treating or preventing a bacterial infection or disease or condition caused by a bacterial infection in a mammal, comprising administering to said mammal a compound of Formula I, IIB, or IIA, or a solvate, metabolite, or pharmaceutically acceptable salt or prodrug thereof. A further aspect of the invention includes methods of treating or preventing a bacterial infection or disease or condition caused by a bacterial infection in a mammal, comprising administering to said mammal a compound of Formula IB or a solvate, metabolite, or pharmaceutically acceptable salt or prodrug thereof.

As used herein, the term “treating” is intended to mean reversing, alleviating, inhibiting the progress of, or preventing a disease or condition in a mammal, such as a human, that has a bacterial infection and includes, but is not limited to, preventing the disease condition from occurring in a mammal, particularly when the mammal is found to be predisposed to having the disease condition but has not yet been diagnosed as having it; modulating and/or inhibiting the disease condition; and/or alleviating the disease condition. The term “treatment,” as used herein, unless otherwise indicated, refers to the act of treating as “treating” is defined immediately above. The terms “treating”, “treat”, or “treatment” embrace both preventative, i.e., prophylactic, and palliative treatment.

The phrase “therapeutically effective amount” means an amount of a compound of the invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

As used herein, unless otherwise indicated, the terms or phrases “bacterial infection(s)” and “disorders related to bacterial infections” include, but are not limited to pneumonia, otitis media, sinusitis, bronchitis, tonsillitis and mastoiditis related to infection by Streptococcus pneumoniae, Haemophilus influenzae, Moraxella catarrhalis, Staphylococcus aureus, Enterococcus faecalis, E. Faecium, E. caaelflavus, S. epidermis, S. haemolyticus, or Peptostreptococcus spp.; pharyngitis, rheumatic fever and glomerulonephritis related to infection by Streptococcus pyogenes, Groups C and G streptococci, Corynebacterium diphtheriae or Actinobacillus haemolyticum; respiratory tract infections related to infection by Mycobacteria spp., Mycoplasma pneumoniae, Legionella pneumophila, Streptococcus pneumoniae, Haemophilus influenzae, or Chlamydia pneumoniae; blood and tissue infections, including edocarditis and osteomyelitis, caused by S. aureus, S. haemolyticus, E. faecalis, E. Faecium, E. durans, including strains resistant to known antibacterials such as, but not limited to, beta-lactams, vancomycin, aminoglycosides, quinolones, chloramphenicol, tetracyclines, streptogramins (such as Synercid), lipopetides (such as Daptomycion), oxazolidinones (such as Linezolide) and macrolides; uncomplicated skin and soft tissue infections and abscesses, and puerperal fever related to infection by Staphylococcus aureus, coagulase-negative staphylococci (i.e., S. epidermis, S. haemolyticus, etc.), Streptococcus pyogenes, Streptococcus agalactiae, Streptococcal groups C-F (minute-colony streptococci), viridans streptococci, Corynebacterium minutissimum, Clostridium spp., or Bartonella henselae; complicated skin and soft tissue infections related to infection by S. aureus, P. aeruginosa, Enterococcus spp., Enterobacter spp.; uncomplicated acute urinary tract infections related to infection by Staphylococcus aureus, coagulase-negative staphylococcal species, or Enterococcus spp.; complicated urinary tract infections related to infection by S. aureus, Pseudomonas spp., Klebsiella spp., Proteus spp., Enterococcus spp., Enterobacter spp.; urethritis and cervicitis; sexually transmitted diseases related to infection by Chlamydia trachomatis, Haemophilus ducreyi, Treponema pallidum, Ureaplasma urealyticum, or Neiserria gonorrheae; toxin diseases related to infection by S. aureus (food poisoning and toxic shock syndrome) or Group A, B and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by Chlamydia trachomatis, Neiserria gonorrheae, S. aureus, S. pneumoniae, S. pyogenes, H. influenzae, Listeria spp., disseminated Mycobacterium avium, or Mycobacterium intracellulare; infections caused by Mycobacterium tuberculosis, M. Leprae, M. paratuberculosis, M. kanasasii, or M. chelonei; gastroenteritis related to infection by Campylobacter jejuni; antibiotic-associated diarrhea; colitis or pseudomembraneous colitis related to infection by Clostridium difficile, Clostridium perfingens, and S. aureous; odontogenic infection by viridans streptococci; persistent cough related to infection by Bordetella pertussis; gas gangrene related to infection by Clostridium peifingens or Bacteroides spp.; and atherosclerosis or cardiovascular disease related to infection by Helicobacter pylori or Chlamydia pneumoniae.

Disorders related to bacterial infections which may be treated or prevented in mammals according to the methods of the invention also include the following: bovine respiratory disease related to infection by P. haemolytica, P. multocida, Mycoplasma bovis, or Bordetella spp.; dairy cow mastitis related to infection by S. aureus, Strep. Uberis, Streptococcus agalactiae, Streptococcus dysgalactiae, Corynebacterium, or Enterococcus spp.; swine respiratory disease related to infection by A. pleuro., P. multocida, or Mycoplasma spp.; swine enteric disease related to infection by Lawsonia intracellularis, Salmonella, or Serpulia hyodysinteriae; cow foot-rot related to infection by Fusobacterium spp.; cow hairy warts related to infection by Fusobacterium necrophorum or Bacteroides nodosus; cow pink eye related infection by Moraxella bovis; skin and soft tissue infections in dogs and cats related to infection by S. epidermis, S. intermedius, coagulase negative Staphylococcus or P. muloticda; and dental and mouth infections in dogs and cats related to infection by Alcaligenes spp., Bacteroides spp., Clostridium spp., Enterobacter spp., Eubacterium, Peptostreptococcus, Porphyromonas, or Prevotella. Other bacterial infections and disorders related to such infections, which may be treated or prevented in accord with the method of the present invention are referred to in J. P. Sanford et al., “The Sanford Guide to Antimicrobial Therapy,” 26^{th} Edition, (Antimicrobial Therapy, Inc., 1996).

The size of the dose for therapeutic or prophylactic purposes of a compound of Formula I, IIB, IIA, or IB will naturally vary according to the nature and severity of the conditions, the age and sex of the animal or patient and the route of administration, according to well known principles of medicine.

The compounds of the present invention may be administered by any route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal. It will be appreciated that the preferred route may vary with for example the condition of the recipient. Where the compound is administered orally, it may be formulated as a pill, capsule, tablet, etc. with a pharmaceutically acceptable carrier or excipient. Where the compound is administered parenterally, it may be formulated with a pharmaceutically acceptable parenteral vehicle and in a unit dosage injectable form, as detailed below.

In order to use a compound of Formula I, IIB, IIA, or IB or a pharmaceutically acceptable salt, solvate, metabolite, prodrug thereof, for the therapeutic treatment (including prophylactic treatment) of mammals including humans, it is normally formulated in accordance with standard pharmaceutical practice as a pharmaceutical composition. According to this aspect of the present invention there is provided a pharmaceutical composition that comprises a compound of Formula I, IIB, IIA, or IB or a solvate, metabolite, or pharmaceutically acceptable salt or prodrug thereof, in association with a pharmaceutically acceptable diluent or carrier.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent) is dissolved in a suitable solvent in the presence of one or more of the excipients described above. The compound of the present invention is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations of the compounds of the present invention may be prepared for various routes and types of administration. For example, a compound of Formula I or IIB having the desired degree of purity may optionally be mixed with pharmaceutically acceptable diluents, carriers, excipients or stabilizers (Remington's Pharmaceutical Sciences (1980) 16-th edition, Osol, A. Ed.), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16-th edition, Osol, A. Ed. (1980). A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes.

The compositions of the present invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, or intramuscular dosing or as a suppository for rectal dosing). For example, compositions intended for oral use may contain, for example, one or more coloring, sweetening, flavoring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the present invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient that is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedures well known in the art.

Compositions for administration by insufflation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

Sustained-release preparations of compounds of Formula I, IIB, IIA, or IB may be prepared. Suitable examples of sustained-release preparations include semipermeable matrices of solid hydrophobic polymers containing a compound of Formula I, IIB, IIA, or IB which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOTT (injectable microspheres composed of lactic acid-glycolic acid copolymer and leuprolide acetate) and poly-D-(−)-3-hydroxybutyric acid.

For further information on formulations, see Chapter 25.2 in Volume 5 of Comprehensive Medicinal Chemistry (Corwin Hansch; Chairman of Editorial Board), Pergamon Press 1990, which is specifically incorporated herein by reference.

The amount of a compound of the present invention that is combined with one or more excipients to produce a single dosage form will necessarily vary depending upon the subject treated, the severity of the disorder or condition, the rate of administration, the disposition of the compound and the discretion of the prescribing physician. However, an effective dosage is in the range of about 0.001 to about 100 mg per kg body weight per day, preferably about 0.1 to about 35 mg/kg/day, in single or divided doses. In some instances, dosage levels below the lower limit of the aforesaid range may be more than adequate, while in other cases still larger doses may be employed without causing any harmful side effect, provided that such larger doses are first divided into several small doses for administration throughout the day. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

A compound of the present invention may be used alone in combination with other drugs and therapies used in the treatment of bacterial infections and disorders related to such infections. For example, a compound of the present invention may be applied in combination with one or more other antibiotics including β-lactams such as carbapenems, cephalosporins (ceftriaxone), and penicillins; aminoglycosides (as exemplified by gentamicin and including but not limited to amikacin, dibekacin, streptomycin, neomycin, kanamycin, spectinomycin, kasugamycin); fluoroquinolones (as exemplified by ciprofloxacin and including but not limited to trovafloxacin, sparfloxacin, gatifloxacin, grepafloxacin, ofloxacin, norfloxacin, floxin, levofloxacin) and related quinolones and naphthyridines with activity against topoisomerases; chloramphenicol; as well as macrolides, ketolides azalides (and other related polyketides), Synercid®, tetracyclines (including glyctlcyclines), glycopeptides (including, but not limited to, vancomycin, teicoplanin, Ortivancin, Telavancin), novobiocin (and coumermycin), lipopeptides (including but not limited to Daptomycin) and oxazolidinones (including but not limited to Linezolid). Such conjoint treatment may be achieved by way of the simultaneous, sequential or separate dosing of the individual components of treatment.

The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

In another embodiment of the present invention, an article of manufacture, or “kit”, containing materials useful for the treatment of the disorders described above is provided. In one embodiment, the kit comprises a container comprising a compound of this invention or a solvate, metabolite, or pharmaceutically acceptable salt or prodrug thereof. The kit may further comprise a label or package insert on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold a compound of this invention or a formulation thereof which is effective for treating the condition and may have a sterile access port (for example, the container may be an intravenous solution bag or a vial having a stopper pierceable by a hypodermic injection needle). At least one active agent in the composition is a compound of this invention. The label or package insert indicates that the composition is used for treating a bacterial infection. Alternatively, or additionally, the kit may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the compound of this invention and, if present, the second pharmaceutical formulation. For example, if the kit comprises a first composition comprising a compound of this invention and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of a compound of this invention, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with a compound of this invention contained therein; and optionally (b) a second container with a second pharmaceutical formulation contained therein, wherein the second pharmaceutical formulation comprises a second compound with antibacterial activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain other embodiments wherein the kit comprises a composition of this invention and a second therapeutic agent, the kit may comprise a container for containing the separate compositions such as a divided bottle or a divided foil packet, however, the separate compositions may also be contained within a single, undivided container. The kit may further comprise directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

Accordingly, another aspect of the present invention provides a kit for treating a bacterial infection, wherein said kit comprises: a) a first pharmaceutical composition comprising a compound of Formula I, IIB, or IIA; and b) instructions for use. In certain embodiments, the kit further comprises: (c) a second pharmaceutical composition, wherein the second pharmaceutical composition comprises a second compound having anti-bacterial activity. In certain embodiments, the kit further comprises instructions for the simultaneous, sequential or separate administration of said first and second pharmaceutical compositions to a patient in need thereof. In certain embodiments, the first and second pharmaceutical compositions are contained in separate containers. In other embodiments, the first and second pharmaceutical compositions are contained in the same container.

### BIOLOGICAL EXAMPLES

The biological activities of the compounds of the present invention were demonstrated by the following assays.

### Example A

### In Vitro Assay 1-a

One assay employs conventional methodology and interpretation criteria and is designed to provide direction for chemical modifications that may lead to compounds with antibacterial activity against susceptible and drug resistant organisms including, but not limited to, beta-lactam, macrolide, and vancomycin resistant organisms. In the assay, a panel of bacterial strains is assembled to include a variety of target pathogenic species including representatives of antibiotic resistant bacteria. Use of this panel enables the chemical structure/activity relationship to be determined with respect to potency and spectrum of activity. The assay was performed in microtiter trays and interpreted according to the guidelines in Performance Standards for Antimicrobial Disk Susceptibility (Sixth Edition; Approved Standard, published by The National Committee for Clinical Laboratory Standards (NCCLS)). The minimum inhibitory concentration (MIC) is used to compare strains. Compounds are initially dissolved in phosphate buffer stock solutions.

### Example B

### In Vitro Assay 1-b

The activity of the compounds of the present invention also may be assessed in accord with Steers replicator technique which is a standard in vitro bacterial testing method described by Steers, et al., 1959, Antibiotics and Chemotherapy, 9:307.

### Example C

### MIC Determination in MRSA

### Reagents:

Cation Adjusted Mueller Hinton Broth (Teknova #M5860); MRSA (Methicillin resistant Staphylococcus aureus, ATCC33591). Test compounds: all compounds were dissolved in PBS at 10 mg/mL and stored at −20° C.

### MIC Protocol:

The method used for determination of MIC's follows the procedure described by the Clinical Laboratory Standards Institute (CLSI). A standardized inoculum of MRSA was prepared per CLSI methods yielding a final cell concentration of 5×10 (5) CFU/mL. 10× compound dilutions were prepared by performing 2 fold serial dilutions in Cation Adjusted Mueller Hinton Broth. The starting and ending concentrations were 640 μg/mL and 1.25 μg/mL, respectively for final assay concentrations of 64 g/mL and 0.125 μg/mL. 10 μL of the 10× compound dilutions were added to 90 μL of the MRSA cell suspension in microplate wells. Plates were incubated in a 35° C. incubator for approximately 20 hours.

### Example D

### In Vivo Assay

The in vivo activity of the compounds of the present invention can be determined by conventional animal protection studies well known to those skilled in the art, usually carried out in rodents.

According to one in vivo model, compounds were evaluated for efficacy in mouse models of acute bacterial infection as follows. Mice (ICR male; 22-26 g) were allotted to APECR cages upon their arrival, and allowed to acclimate for at least 1 week before being placed in a study. All animals were maintained in a hygienic environment under a controlled temperature (22-23° C.) and humidity (50%-60%) with 12 hours light/dark cycles for at least one week. Free access to standard lab chow (LabDiet Rodent Diet, PMI International, USA) and tap water was granted. The acute infection was produced by intravenous inoculation with an LD₉₀₋₁₀₀ dose of bacteria (Staphylococcus aureus methicillin resistant strain ATCC 33591, 8.0×10⁷ CFU/mouse) suspended in 0.2 mL of phosphate buffer pH 7.4 without 5% mucin.

Mice (10 per group) were treated subcutaneously at 1 hour after challenge. Appropriate non-treated (infected but not treated) and positive (vancomycin or minocycline) controls were included in each study. Percent survival was recorded once daily for 10 days. At the end of 10 days of dosing, an increase of survival by 50 percent or more relative to vehicle control indicated a significant effect. The PD₅₀ (mg/kg/dose calculated to protect 50% of infected animals) is determined by the probit method.

### Example E

### In Vivo Neurotoxicity Assay

An in vivo 14 day toxicity study was performed in order to evaluate the potential for compounds of Formula IIB to cause dose-related sciatic nerve demyelination after subacute dosing. 50 Male CD-1 mice were treated according to the protocol described below for 14 days of treatment for all animals; 50% of the animals were retained for a 14-day non-dosing recovery.

Dosing protocol: Group 1: Saline, SC, 10 ml/kg, QD×14; Group 2: compounds of Formula IIB, SC, 10 mg/kg, QD×14; Group 3: compounds of Formula IIB, SC, 30 mg/kg, QD×14.

After treatment, the sciatic nerves removed, fixed in formalin, and submitted for histopathology evaluations. Each sciatic nerve was trimmed (one cross section and one longitudinal section), processed, embedded, sectioned onto glass slides and stained with luxol fast blue. A qualified veterinary pathologist provided a pathology interpretation for demyelination. There was no demyelination in any animal treated with certain compounds of Formula IIB.

### PREPARATIVE EXAMPLES

In order to illustrate the present invention, the following examples are included. However, it is to be understood that these examples do not limit the present invention and are only meant to suggest a method of practicing the present invention. Persons skilled in the art will recognize that the chemical reactions described may be readily adapted to prepare a number of other capreomycin analogs of the present invention, and alternative methods for preparing the compounds of the present invention are deemed to be within the scope of the present invention. For example, the synthesis of non-exemplified compounds according to the present invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the present invention.

In the examples described below, unless otherwise indicated all temperatures are set forth in degrees Celsius. Reagents were purchased from commercial suppliers such as Aldrich Chemical Company, Lancaster, TCI or Maybridge, and were used without further purification unless otherwise indicated. Tetrahydrofuran (THF), N,N-dimethylformamide (DMF), dichloromethane, toluene, dioxane and 1,2-difluoroethane were purchased from Sigma-Aldrich in Sure seal bottles and used as received. Capreomycin sulfate was purchased from Sigma-Aldrich.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

Column chromatography was done on a Biotage system (Manufacturer: Dyax Corporation) having a silica gel column or on a silica SepPak cartridge (Waters).

### Example 1

### Preparation of Compound Mixture 1

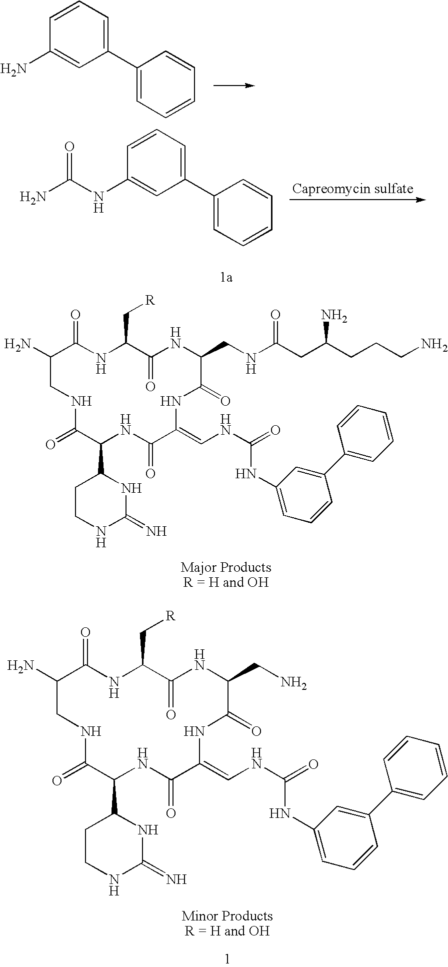

Step A: 20.0 g of 3-aminobiphenyl was dissolved in a solution of 250 mL of acetic acid and 200 mL of water under an atmosphere of dry N₂. To this mixture was added, portionwise, 13.42 g of potassium cyanate. The reaction mixture was stirred for about 2 hours after which time it was diluted with 500 mL of water. The precipitate was collected via suction filtration and was then washed with water and finally air-dried to give a white solid as the desired product. The solid was taken up in 200 mL of ethyl ether and stirred at room temperature for about 1 hour after which time it was filtered. The resulting solid was then taken up in 200 mL of isopropanol and stirred at room temperature for about 1 hour and then filtered. The solid was washed with ethyl ether and then air-dried to give 17.7 g of 3-biphenylurea as a white solid. The material was used in Step B without further purification.

Step B: Capreomycin sulfate (16.3 g) and 3-biphenylurea (27.7 g) were dissolved/suspended in 300 mL of a 1:1 solution of dioxane/1 N aqueous HCl. The reaction mixture was heated at 65° C. overnight. The reaction mixture was cooled to room temperature and filtered, and the resulting filtrate was adjusted to ˜pH 7.2 with the addition of NaHCO₃ and then diluted with 200 mL of saturated brine. The mixture was then partitioned between ethyl acetate and water. The water layer was washed 5 times with ethyl acetate, and then 650 g of activated Amberlite XAD-16 resin (activated by stirring in methanol 20 minutes, filtering and washing with water) was added to the water layer and the resulting heterogeneous mixture was stirred for about 2 hours. The resin was then collected via suction filtration and subsequently washed several times with water. The resin was transferred to a beaker to which was added about 600 mL of methanol/water (9:1), and the resulting slurry was stirred for about 30 minutes. The resin was then filtered and the previous step was repeated. The filtrates were combined and then concentrated under vacuum to give 11.59 g of compound mixture 1 as HCl salts (major products MH+=821, 805; minor products MH+=676, 692).

### Example 2

### Preparation of Compound Mixture 2

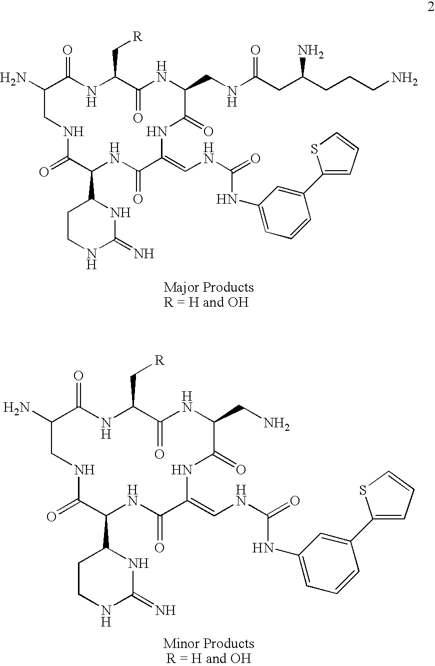

Step A: 3-Bromopheylurea (10.40 g), 2-thiopheneboronic acid (8.20 g) and palladium tetrakistriphenylphosphine (2.63 g) were added to a flask containing 300 mL of dimethoxyethane and 100 mL of 2.0 M aqueous potassium carbonate under an atmosphere of dry N₂. The reaction mixture was heated at 90° C. overnight. The reaction mixture was then cooled to room temperature and partitioned between EtOAc and water. The EtOAc layer was dried over MgSO₄, filtered and concentrated under vacuum. The resulting residue was tritutrated with ethyl ether to give 9.00 g of 1-(3-(thiophen-2-yl)phenyl)urea.

Step B: Capreomycin was reacted with 1-(3-(thiophen-2-yl)phenyl)urea according to the procedure of Example 1 to give compound mixture 2 as HCl salts (major products MH+=811, 827; minor products MH+=682, 698).

### Example 3

### Preparation of Compound Mixture 3

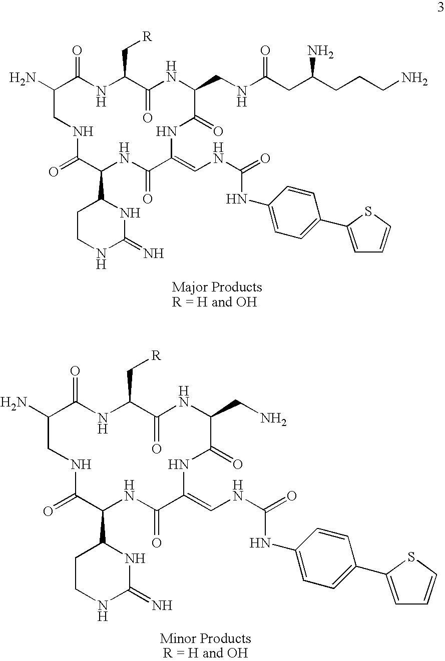

Step A: 10.1 g of 4-iodoaniline, 7.60 g of 2-thiopheneboronic acid, and 2.40 g of palladium tetrakistriphenylphosphine were dissolved/suspended in a solution of 150 mL of dimethoxyethane and 68 mL of 2.0 M aqueous potassium carbonate under an atmosphere of dry N₂. The reaction mixture was heated to 90° C. and reacted at this temperature over night. The reaction mixture was then cooled to room temperature and then partitioned between EtOAc and water. The EtOAc layer was then dried over MgSO₄, filtered and concentrated under vacuum. The resulting residue was purified using silica gel chromatography eluting with a 2:1 solution of DCM/Hexanes and then DCM to give 2.80 g of 1-(4-(thiophen-2-yl)phenyl)urea.

Step B: Capreomycin sulfate and 1-(4-(thiophen-2-yl)phenyl)urea were reacted according to the method of Example 1, Step B to provide compound mixture 3 (major products MH+=821, 805; minor products MH+=676, 692).

### Example 4

### Preparation of Compound Mixture 4

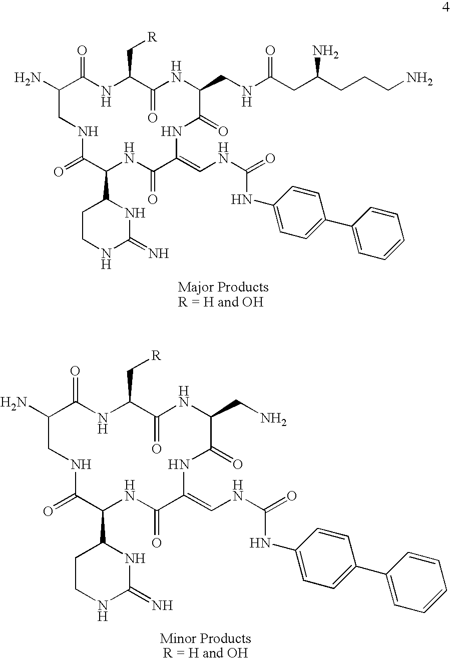

Compound mixture 4 was prepared according to the method of Example, Steps A and B, except that 4-aminobiphenyl was substituted for 3-aminobiphenyl, to provide the desired compounds as HCl salts (major products MH+=821, 805; minor products MH+=676, 692).

### Example 5

### Preparation of Compound Mixture 5

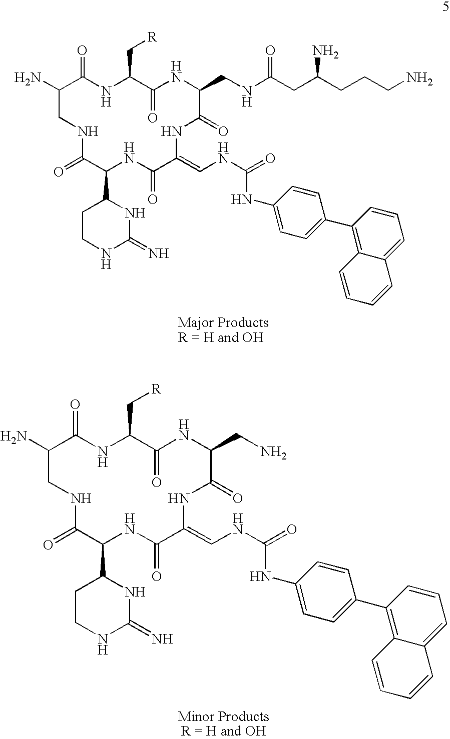

Compound mixture 5 was prepared according to Example 3, except that 1-napthaleneboronic acid was substituted for 2-thiopheneboronic acid, to provide the desired products (major products MH+=855, 871; minor products MH+=727, 743).

### Example 6

### Preparation of Compound Mixture 6

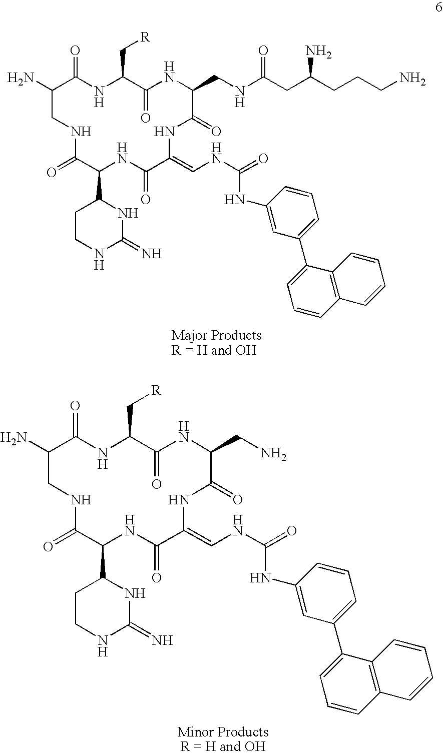

Compound mixture 6 was prepared as an HCl salt according to the procedure outlined in Example 3, substituting 2-thiopheneboronic acid with 1-napthaleneboronic acid and substituting 4-iodoaniline with 3-iodoaniline (major products MH+=855, 871; minor products MH+=727, 743).

### Example 7

### Preparation of Compound Mixture 7

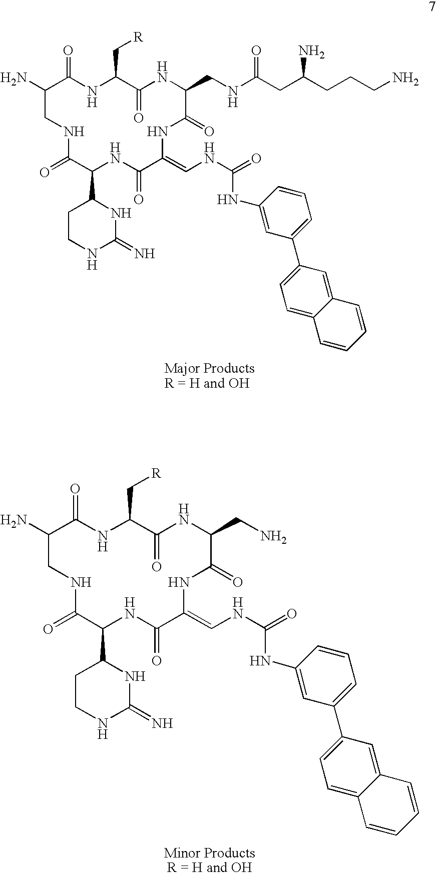

Compound mixture 7 was prepared as an HCl salt according to the procedure outlined in Example 3, substituting 2-thiopheneboronic acid with 2-napthaleneboronic acid and substituting 4-iodoaniline with 3-iodoaniline (major products MH+=855, 871; minor products MH+=727, 743).

### Example 8

### Preparation of Compound Mixture 8

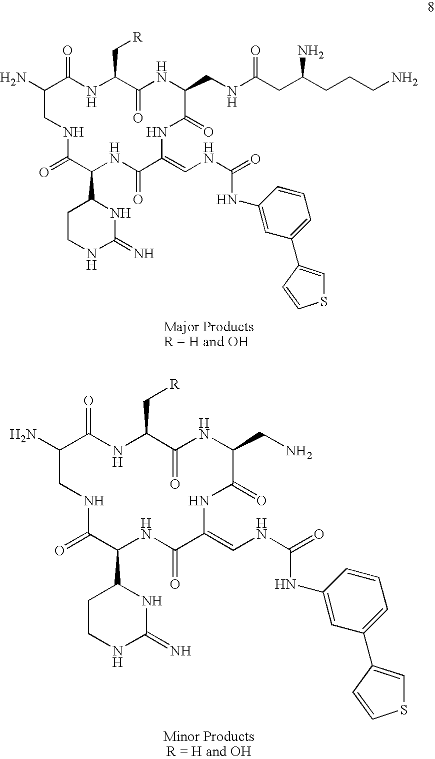

Compound mixture 8 was prepared as an HCl salt according to the procedure outlined in Example 3, substituting 2-thiopheneboronic acid with 3-thiopheneboronic acid and substituting 4-iodoaniline with 3-iodoaniline (major products MH+=811, 827; minor products MH+=683, 699).

### Example 9

### Preparation of Compound Mixture 9

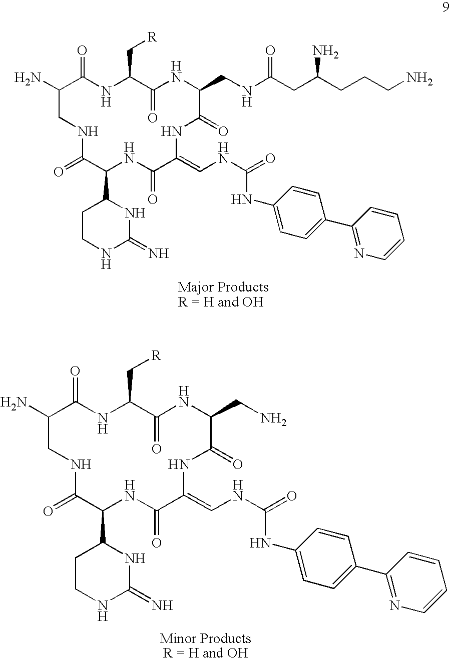

Step A: 2-Bromopyridine (650 μL), 4-nitrophenylboronic acid (1.479 g) and palladium tetrakistriphenylphosphine (391.5 mg) were added to a mixture of 40 mL of dimethoxyethane and 10 mL of 2.0 M aqueous potassium carbonate under an atmosphere of dry N₂. The reaction mixture was heated at 95° C. overnight. The reaction mixture was then cooled to room temperature and diluted with water (˜200 mL). The resulting precipitate was collected, washed with water and air-dried. The dried material was purified using silica gel chromatography to give 1.40 g of 2-(4-nitrophenyl)pyridine.

Step B: 2-(4-Nitrophenyl)pyridine (1.10 g) and 10% Pd/C (300 mg) were dissolved suspended in 80 mL of an 8:1 solution of 30 mL of THF and 10 mL of ethanol under an atmosphere of dry N₂. To this solution was added 1.0 mL of anhydrous hydrazine. The reaction mixture was stirred at room temperature overnight and then filtered. The filtrate was concentrated under vacuum. The residue was partitioned between dichloromethane and water and the organic layer was washed with brine, dried over MgSO₄, filtered and concentrated under vacuum. The residue was purified via silica gel chromatography to give 653.3 mg of 4-(pyridin-2-yl)benzenamine.

Step C: Compound mixture 9 was prepared as the HCl salt according to the procedure outlined in Example 1, except that 3-aminobiphenyl was replaced with 4-(pyridin-2-yl)benzenamine (major products MH+=806, 822; minor products MH+=678, 694).

### Example 10

### Preparation of Compound Mixture 10

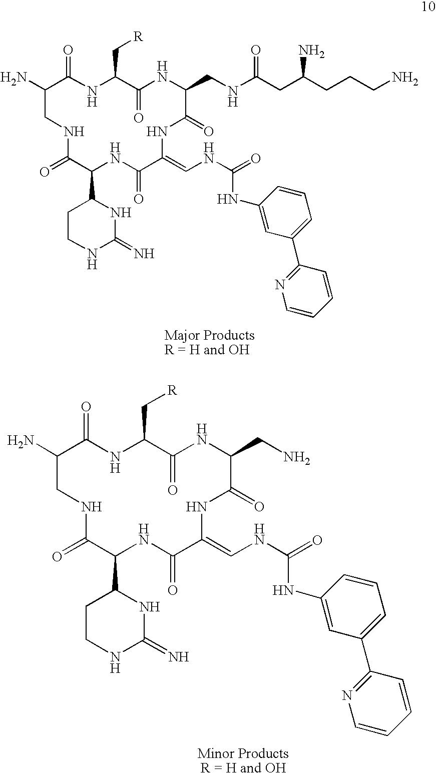

Step A: 2-Bromopyridine (2.00 mL), 3-aminophenylboronic acid (5.40 g) and palladium tetrakistriphenylphosphine (1.2512 g) were added to a mixture of 130 mL of dimethoxyethane and 32 mL of 2.0 M aqueous potassium carbonate under an atmosphere of dry N₂. The reaction mixture was heated at 90° C. overnight. The reaction mixture was then cooled to room temperature and diluted with water (300 mL). The resulting precipitate was collected, washed with water and air-dried. The dried material was purified using silica gel chromatography to give 2.75 g of 3-(pyridin-2-yl)benzenamine.

Step B: Compound mixture 10 was prepared as an HCl salt according to the procedure outlined in Example 1, except that 3-aminobiphenyl was replaced with 3-(pyridin-2-yl)benzenamine (major products MH+=806, 822; minor products MH+=678, 694).

### Example 11

### Preparation of Compound Mixture 11

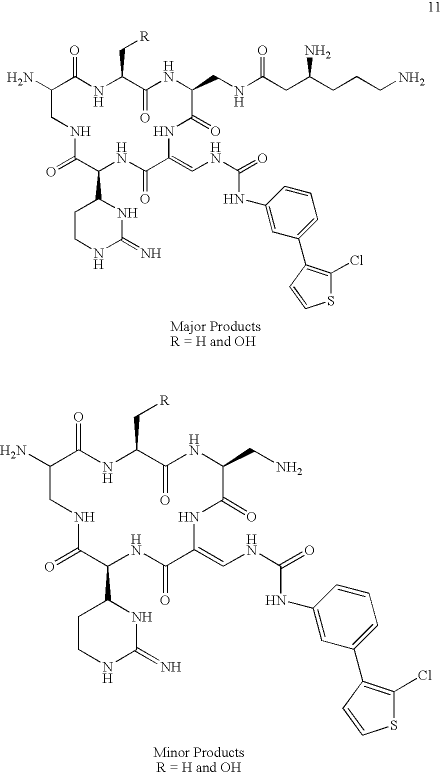

Step A: 3-bromo-2-chlorothiophene (1.00 mL), 3-aminophenylboronic acid (2.2266 g) and palladium tetrakistriphenylphosphine (544.8 mg) were combined in a mixture of 40 mL of dimethoxyethane and 15 mL of 2.0 M aqueous potassium carbonate under an atmosphere of dry N₂. The reaction mixture was heated at 90° C. overnight. The reaction mixture was then cooled to room temperature and partitioned between EtOAc and water. The EtOAc layer was then dried over MgSO₄, filtered and concentrated under vacuum. The resulting residue was purified using silica gel chromatography eluting with a 1:1 solution of DCM/Hexanes, followed by a 4:3 solution of DCM/Hexanes and finally a 2:1 solution of DCM/Hexanes, to give 1.80 g of 3-(2-chlorothiophen-3-yl)benzenamine.

Step B: Compound mixture 11 was prepared as an HCl salt according to the procedure outlined in Example 1, substituting 3-aminobiphenyl with 3-(2-chlorothiophen-3-yl)benzenamine (major products MH+=845, 861; minor products MH+=717, 733).

### Example 12

### Preparation of Compound Mixture 12

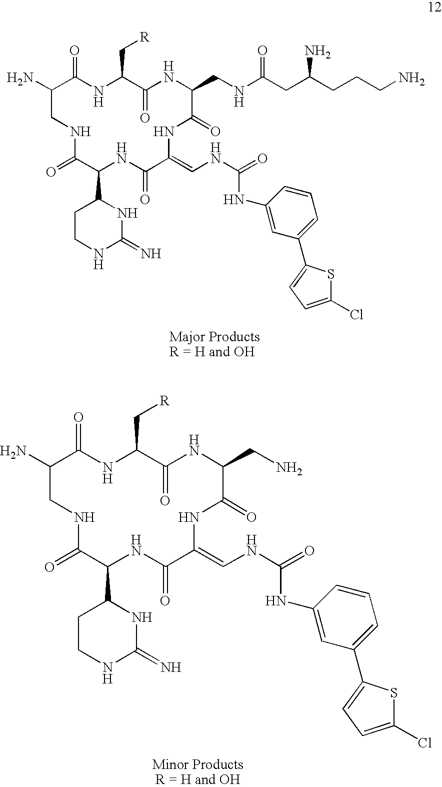

Compound mixture 12 was prepared as an HCl salt according to the procedure outlined in Example 11, except that 3-bromo-2-chlorothiophene was replaced with 2-bromo-5-chlorothiophene (major products MH+=845, 861; minor products MH+=717, 733).

### Example 13

### Preparation of Compound Mixture 13

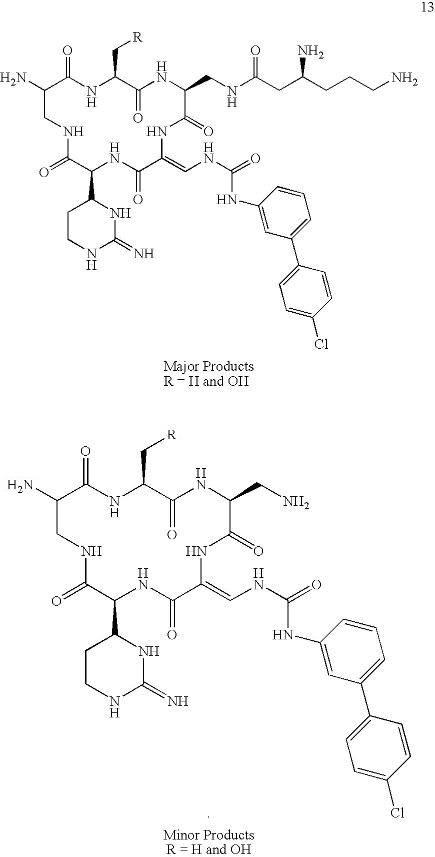

Compound mixture 13 was prepared as an HCl salt according to the procedure outlined in Example 1, except that 3-aminobiphenyl is replaced with 4′-chloro-3-aminobiphenyl (major products MH+=839.4, 855.3; minor products MH+=711.3, 727.3).

### Example 14

### Preparation of Compound Mixture 14

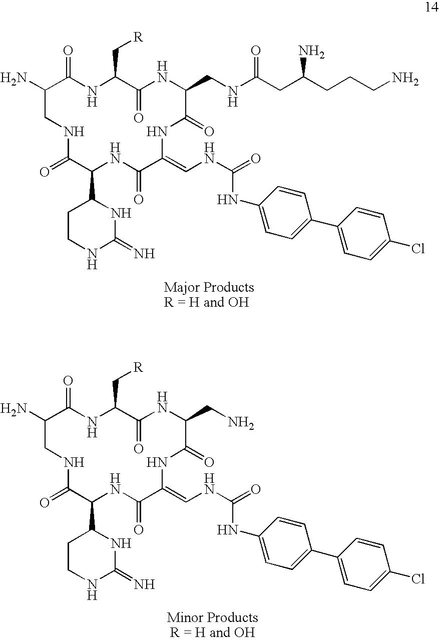

Compound mixture 14 was prepared as an HCl salt according to the procedure outlined in Example 1, except that 3-aminobiphenyl is replaced with 4′-chloro-4-aminobiphenyl (major products MH+=839.4, 855.4; minor products MH+=711.3, 727.3).

### Example 15

### Preparation of Compound Mixture 15

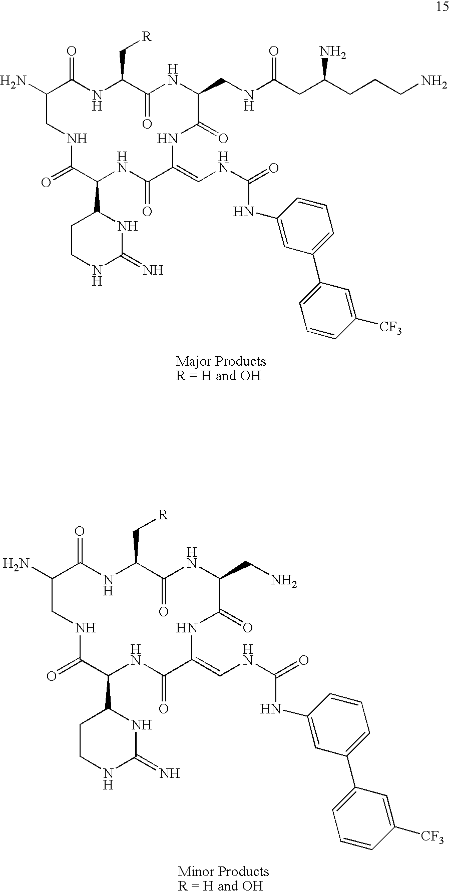

Compound mixture 15 was prepared as an HCl salt according to the procedure outlined in Example 1, except that 3-aminobiphenyl is replaced with 3′-trifluoromethyl-3-aminobiphenyl (major products MH+=873.4, 889.4; minor products MH+=745.4, 761.3).

### Example 16

### Preparation of Compound Mixture 16

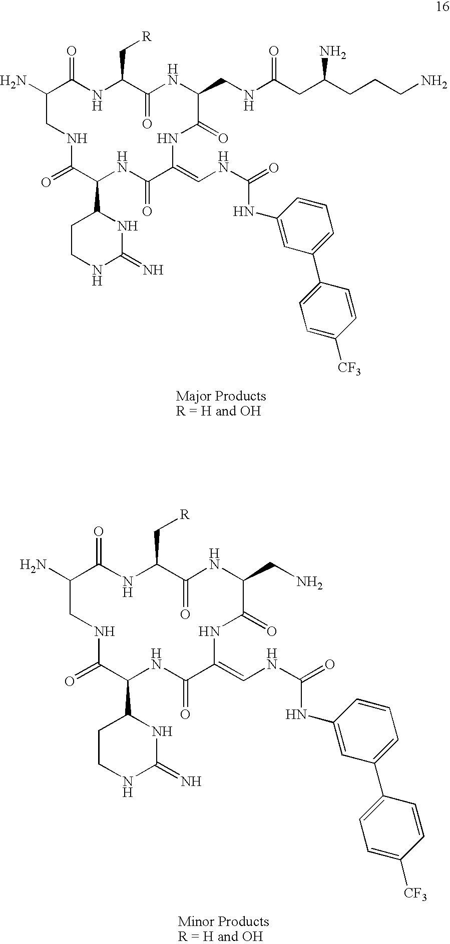

Compound mixture 16 was prepared as an HCl salt according to the procedure outlined in Example 1, except that 3-aminobiphenyl is replaced with 4′-trifluoromethyl-3-aminobiphenyl (major products MH+=873.4, 889.3; minor products MH+=745.4, 761.3).

### Example 17

### Preparation of Compound Mixture 17

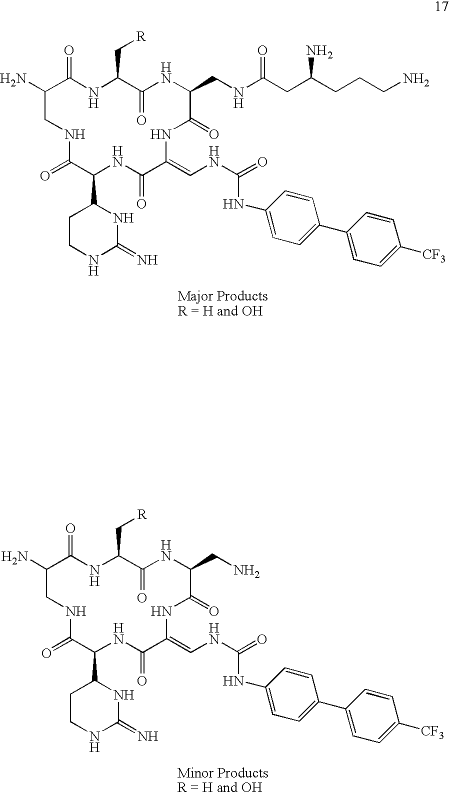

Compound mixture 17 was prepared as an HCl salt according to the procedure outlined in Example 1, except that 3-aminobiphenyl is replaced with 4′-trifluoromethyl-4-aminobiphenyl (major products MH+=873.4, 889.4; minor products MH+=745.4, 761.3).

### Example 18

### Preparation of Compound Mixture 18

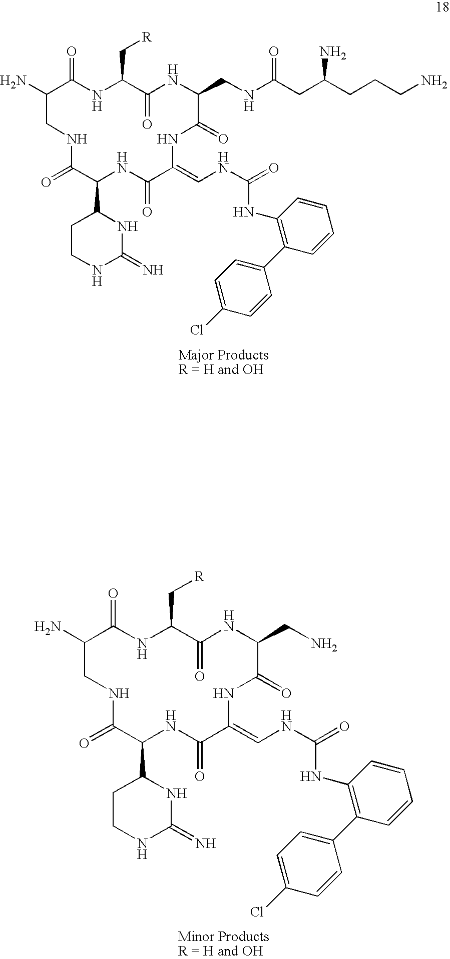

Compound mixture 18 was prepared as an HCl salt according to the procedure outlined in Example 1, except that 3-aminobiphenyl is replaced with 4′-chloro-2-aminobiphenyl (major products MH+=839.4, 855.3; minor products MH+=711.3, 727.3).

### Example 19

### Preparation of Compound Mixture 19

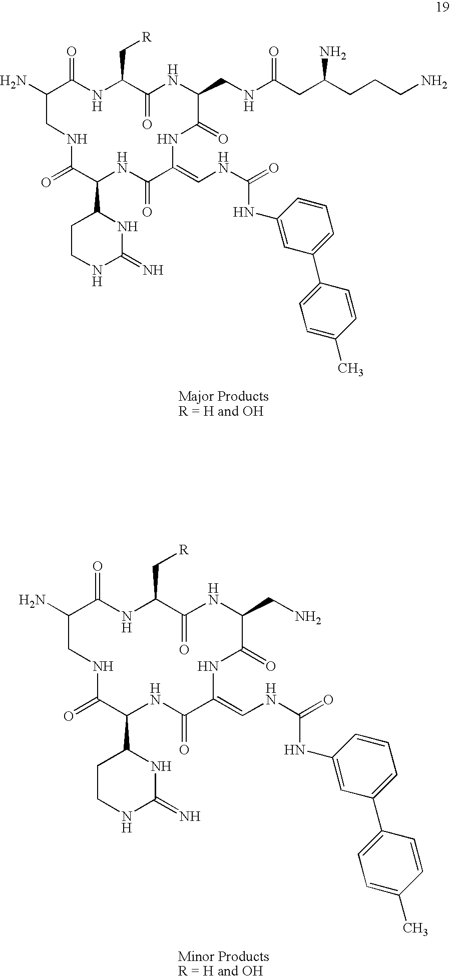

Compound mixture 19 was prepared as an HCl salt according to the procedure outlined in Example 1, except that 3-aminobiphenyl is replaced with 4′-methyl-3-aminobiphenyl (major products MH+=819.4, 835.4; minor products MH+=691.3, 707.3).

### Example 20

### Preparation of Compound Mixture 20

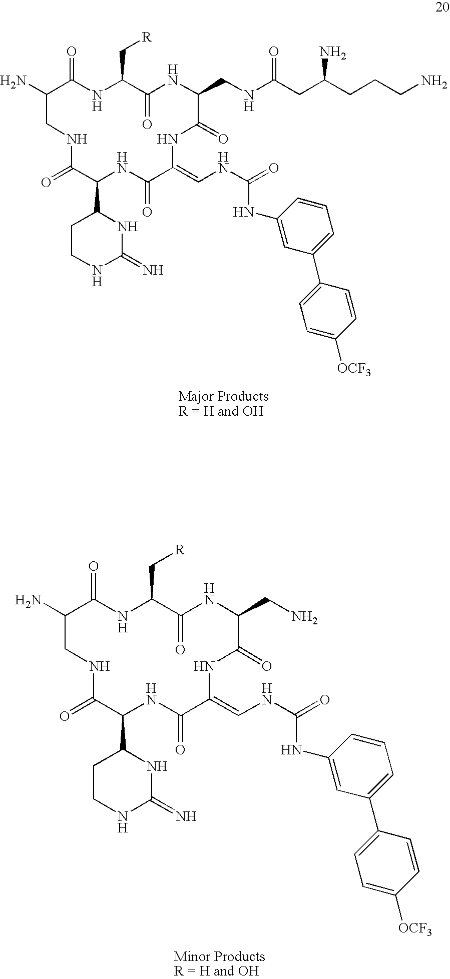

Compound mixture 20 was prepared as an HCl salt according to the procedure outlined in Example 1, except that 3-aminobiphenyl is replaced with 4′-trifluoromethoxy-3-aminobiphenyl (major products MH+=889.4, 905.3; minor products MH+=761.3, 777.3).

### Example 21

### Preparation of Compound Mixture 21

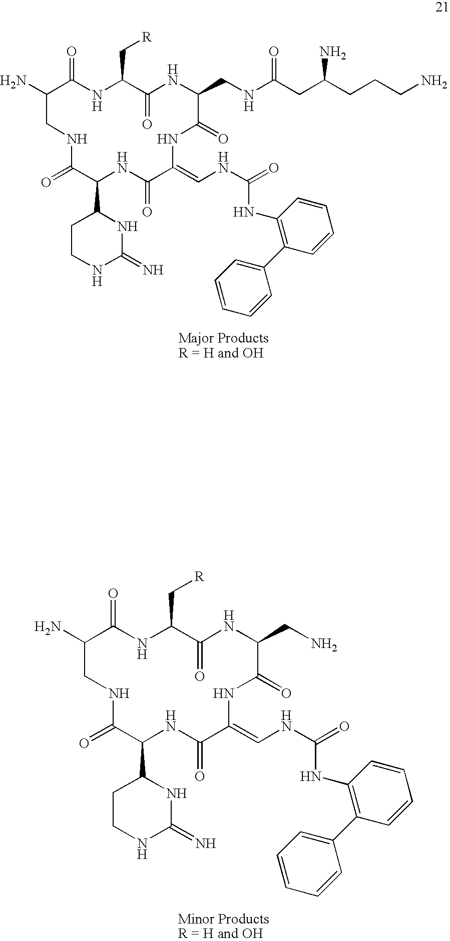

Compound mixture 21 was prepared as an HCl salt according to the procedure outlined in Example 1, except that 3-aminobiphenyl is replaced with 2-aminobiphenyl (major products MH+=805.4, 821.4; minor products MH+=677.3, 693.3).

### Example 22

### Preparation of Compound Mixture 22

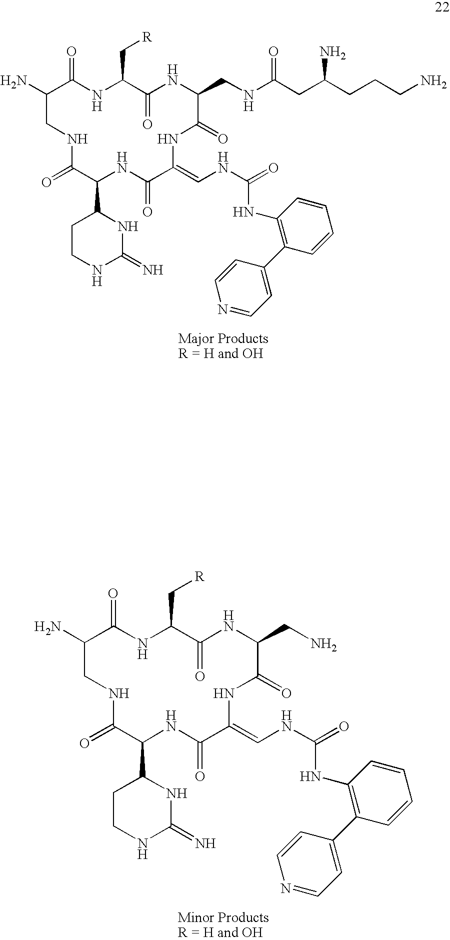

Compound mixture 22 was prepared as an HCl salt according to the procedure outlined in Example 2, except that 2-thiopheneboronic acid is replaced with pyridine-3-boronic acid (major products MH+=806.4, 822.4).

### Example 23

### Preparation of Compound Mixture 23

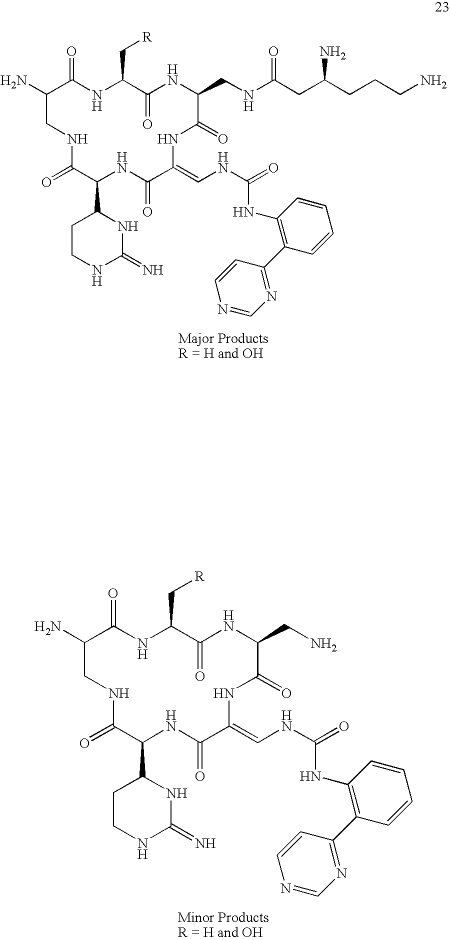

Compound mixture 23 was prepared as an HCl salt according to the procedure outlined in Example 2, except that 2-thiopheneboronic acid is replaced with pyrimidine-5-boronic acid (major products MH+=807.4, 823.4).

### Example 24

### Preparation of Compound Mixture 24

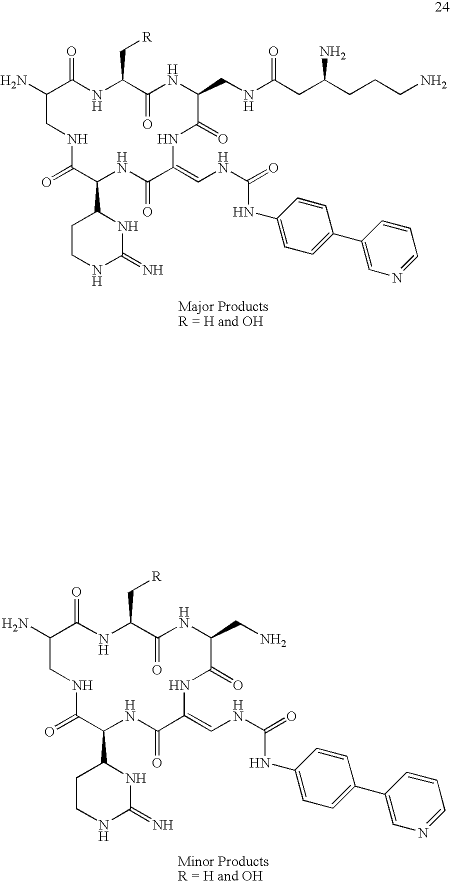

Compound mixture 24 was prepared as an HCl salt according to the procedure outlined in Example 2, except that 2-thiopheneboronic acid is replaced with pyridine-3-boronic acid and 3-bromopheylurea is replaced by 4-bromopheylurea (major products MH+=806.4, 822.4).

### Example 25

### Preparation of Compound Mixture 25

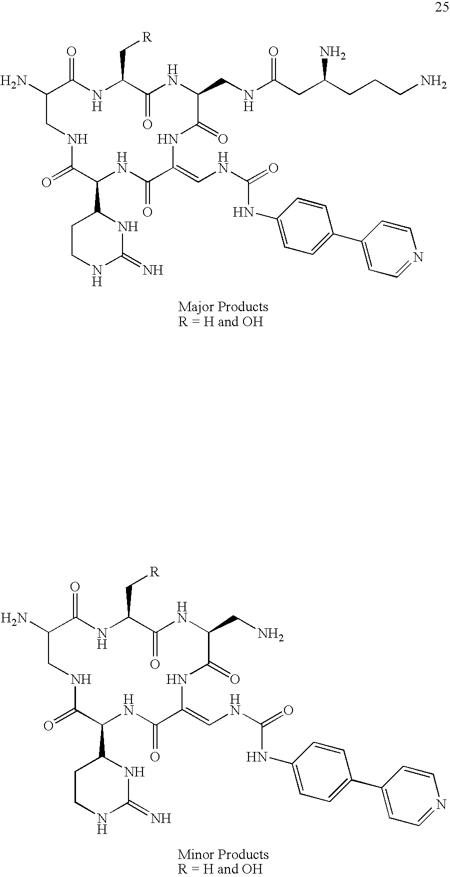

Compound mixture 25 was prepared as an HCl salt according to the procedure outlined in Example 2, except that 2-thiopheneboronic acid is replaced with pyridine-4-boronic acid and 3-bromopheylurea is replaced by 4-bromopheylurea (major products MH+=806.4, 822.4).

### Example 26

### Preparation of Compound Mixture 26

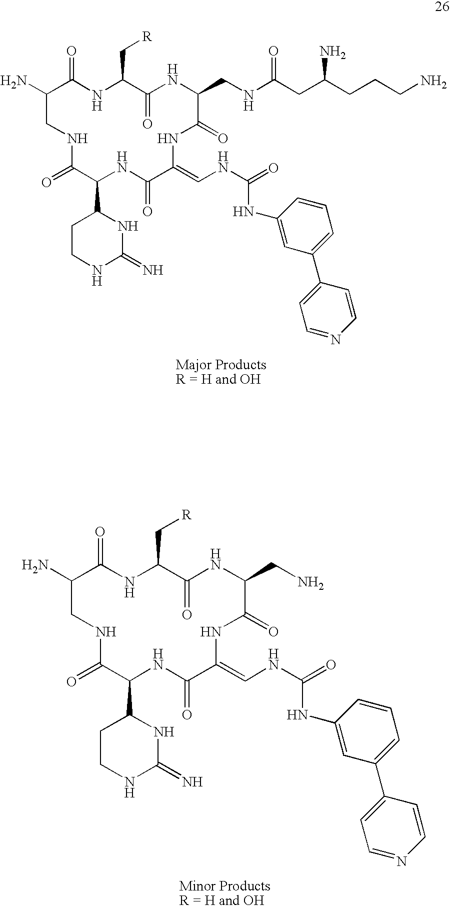

Compound mixture 26 was prepared as an HCl salt according to the procedure outlined in Example 2, except that 2-thiopheneboronic acid is replaced with pyridine-4-boronic acid (major products MH+=806.4, 822.4).

### Example 27

### Preparation of Compound Mixture 27

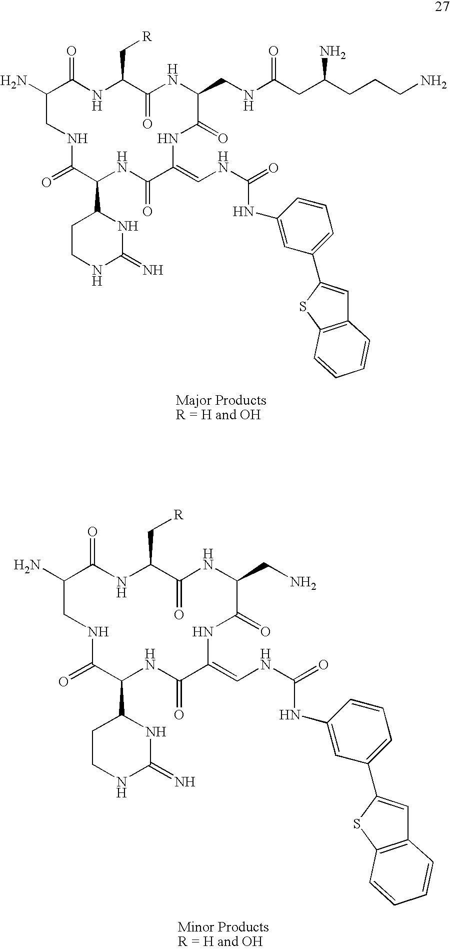

Compound mixture 27 was prepared as an HCl salt according to the procedure outlined in Example 2, except that 2-thiopheneboronic acid is replaced with benzothiophene-2-boronic acid (major products MH+=861.4, 877.3).

### Example 28

### Preparation of Compound Mixture 28

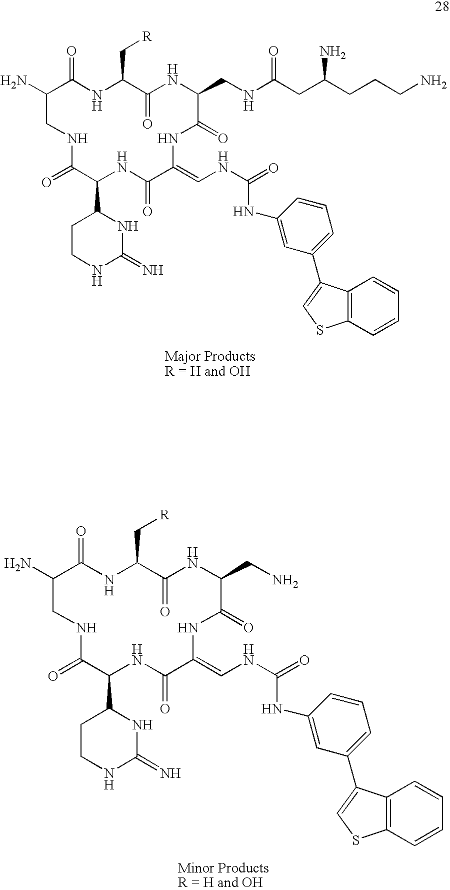

Compound mixture 28 was prepared as an HCl salt according to the procedure outlined in Example 2, except that 2-thiopheneboronic acid is replaced with benzothiophene-3-boronic acid (major products MH+=861.4, 877.3).

### Example 29

### Preparation of Compound Mixture 29

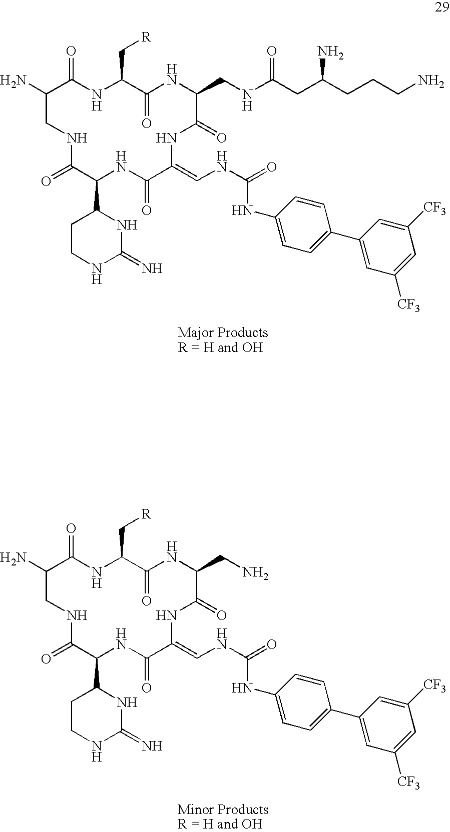

Compound mixture 29 was prepared as an HCl salt according to the procedure outlined in Example 1, except that 3-aminobiphenyl is replaced with 3′,5′-bis-trifluoromethyl-4-aminobiphenyl (major products MH+=941.4, 957.3).

### Example 30

### Preparation of Compound Mixture 30

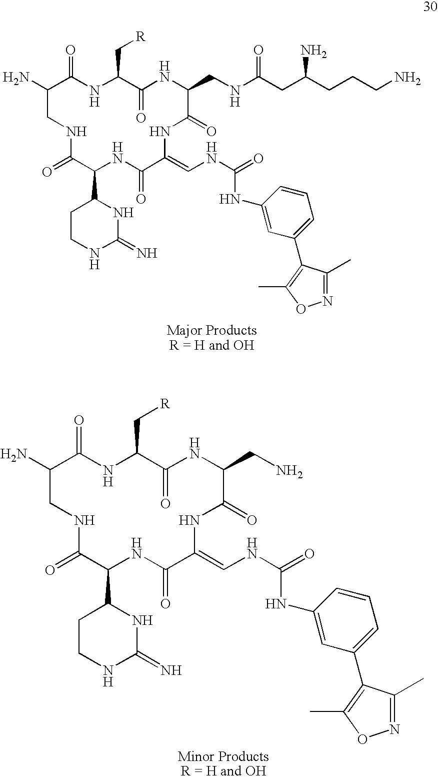

Compound mixture 30 was prepared as an HCl salt according to the procedure outlined in Example 2, except that 2-thiopheneboronic acid is replaced with 3,5-dimethylisoxazol-4-ylboronic acid (major products MH+=824.4, 840.4).

### Example 31

### Preparation of Compound Mixture 31

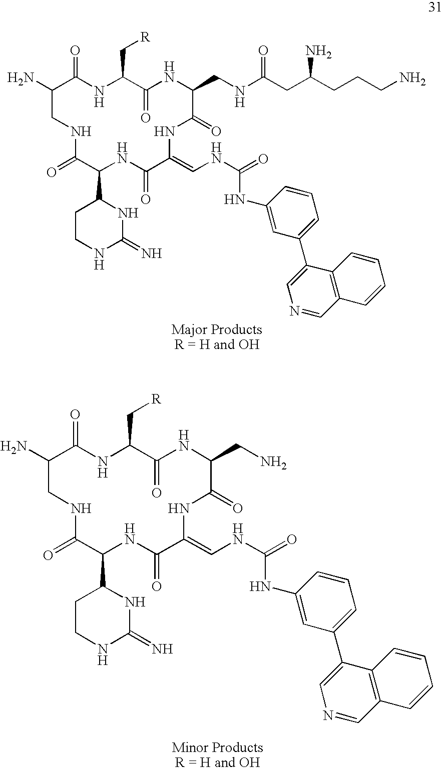

Compound mixture 31 was prepared as an HCl salt according to the procedure outlined in Example 2, except that 2-thiopheneboronic acid is replaced with isoquinolin-4-ylboronic acid (major products MH+=856.4, 872.3).

### Example 32

### Preparation of Compound Mixture 32

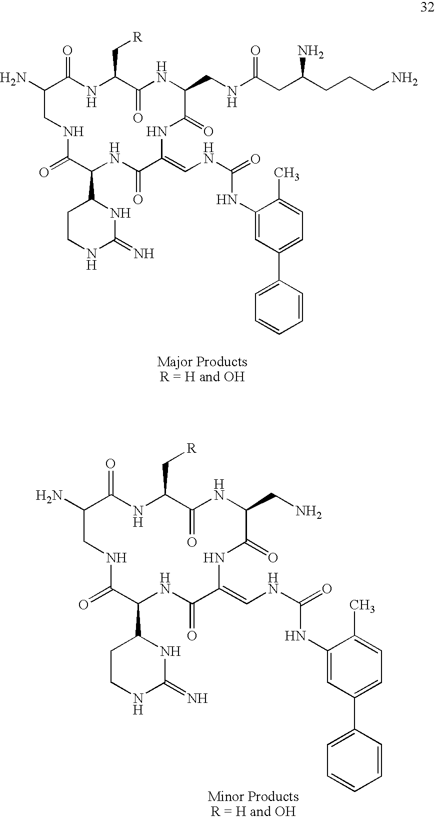

Compound mixture 32 was prepared as an HCl salt according to the procedure outlined in Example 1, except that 3-aminobiphenyl is replaced with 4-methyl-3-aminobiphenyl (major products MH+=819.5, 835.4).

### Example 33

### Preparation of Compound Mixture 33

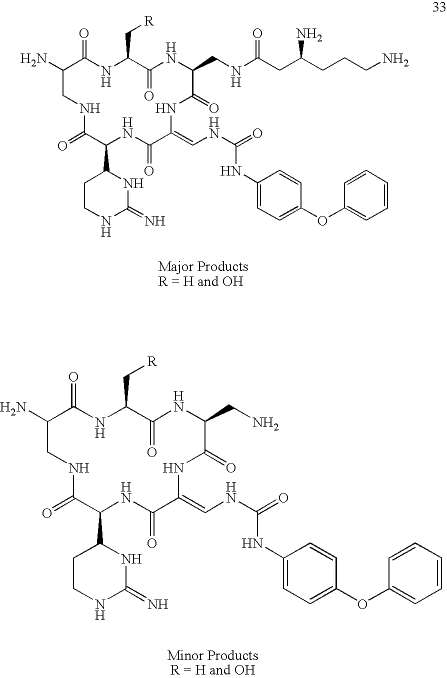

Compound mixture 33 was prepared as an HCl salt according to the procedure outlined in Example 1, except that 3-aminobiphenyl is replaced with 4-phenoxyaniline (major products MH+=821.4, 837.3).

### Example 34

### Preparation of Compound Mixture 34

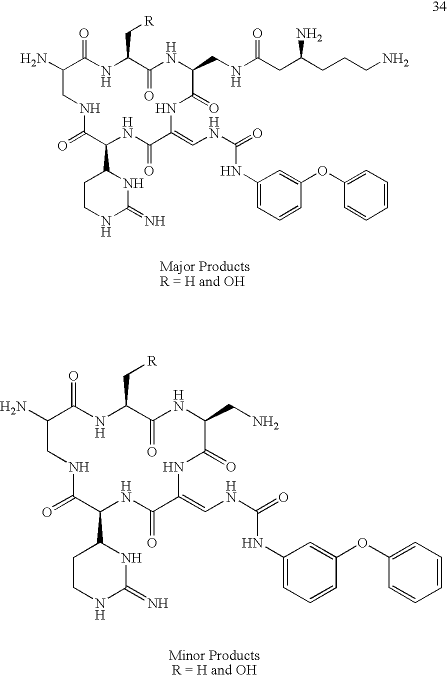

Compound mixture 34 was prepared as an HCl salt according to the procedure outlined in Example 1, except that 3-aminobiphenyl is replaced with 3-phenoxyaniline (major products MH+=821.4, 837.3).

### Example 35

### Preparation of Compound Mixture 35

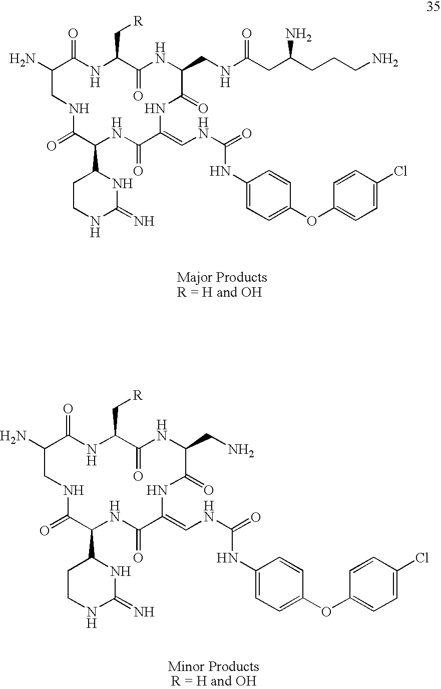

Compound mixture 35 was prepared as an HCl salt according to the procedure outlined in Example 1, except that 3-aminobiphenyl is replaced with 4-(4-chlorophenoxy)-aniline (major products MH+=855.4, 873.3).

### Example 36

### Preparation of Compound Mixture 36

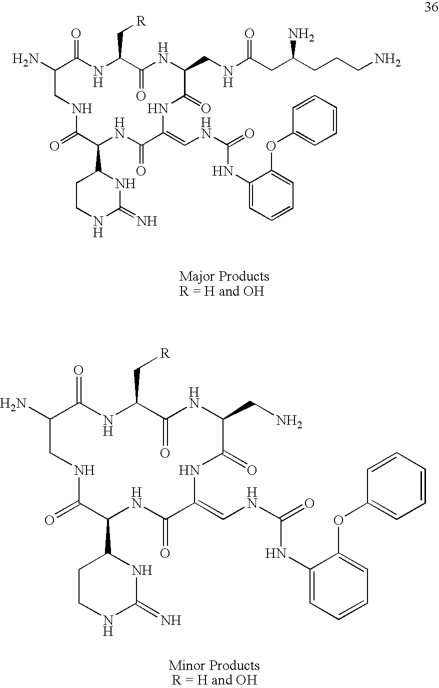

Compound mixture 36 was prepared as an HCl salt according to the procedure outlined in Example 1, except that 3-aminobiphenyl is replaced with 2-phenoxyaniline (major products MH+=821.4, 837.3).

### Example 37

### Preparation of Compound Mixture 37

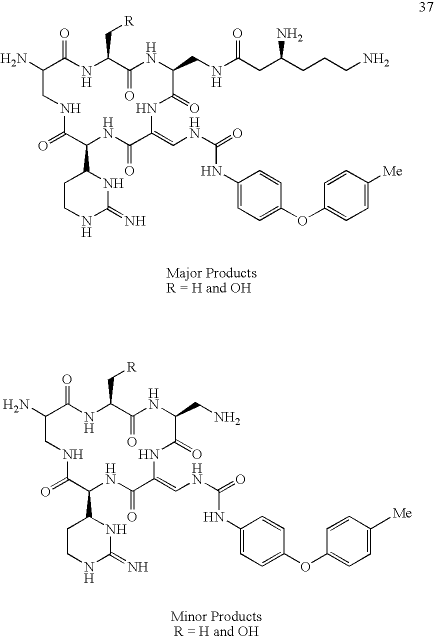

Compound mixture 37 was prepared as an HCl salt according to the procedure outlined in Example 1, except that 3-aminobiphenyl is replaced with 4-(4-methylphenoxy)-aniline (major products MH+=835.4, 851.4).

### Example 38

### Preparation of Compound Mixture 38

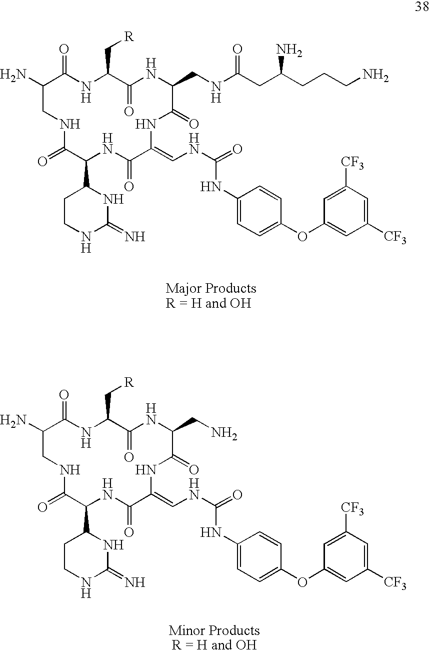

Compound mixture 38 was prepared as an HCl salt according to the procedure outlined in Example 1, except that 3-aminobiphenyl is replaced with 4-(3,5-bis-trifluoromethylphenoxy)-aniline (major products MH+=957.4, 973.3).

### Example 39

### Preparation of Compound Mixture 39

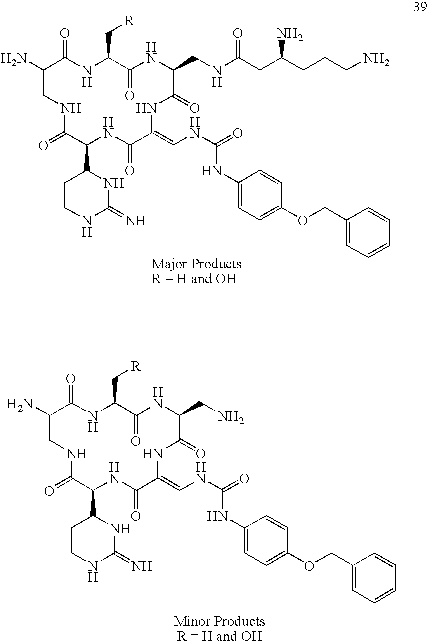

Compound mixture 39 was prepared as an HCl salt according to the procedure outlined in Example 1, except that 3-aminobiphenyl is replaced with 4-benzyloxyaniline (major products MH+=835.4, 851.3).

### Example 40

### Preparation of Compound Mixture 40

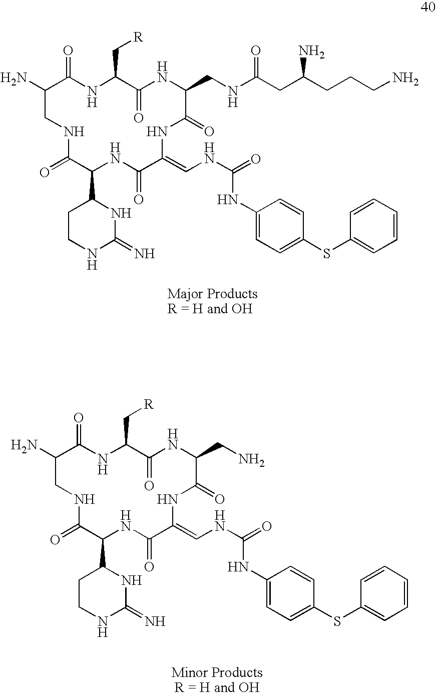

Compound mixture 40 was prepared as an HCl salt according to the procedure outlined in Example 1, except that 3-aminobiphenyl is replaced with 4-(phenylthio)benzenamine (major products MH+=837.5, 853.5).

### Example 41

### Preparation of Compound Mixture 41

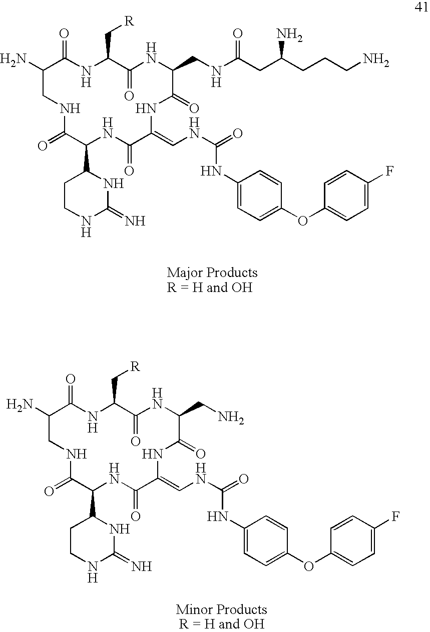

Compound mixture 41 was prepared as an HCl salt according to the procedure outlined in Example 1, except that 3-aminobiphenyl is replaced with 4-(4-fluorophenoxy)-aniline (major products MH+=839.5, 855.5).

### Example 42

### Preparation of Compound Mixture 42

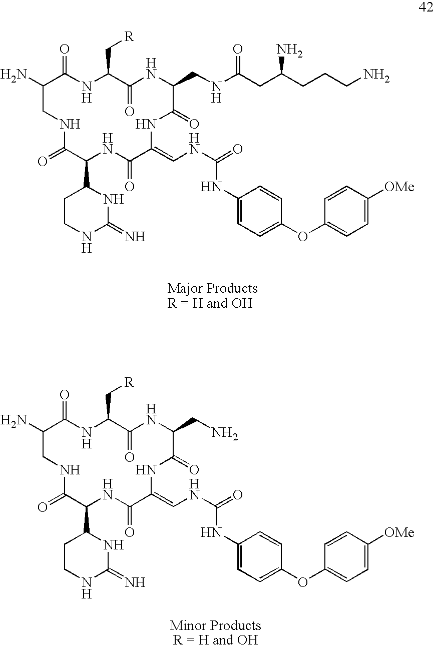

Compound mixture 42 was prepared as an HCl salt according to the procedure outlined in Example 1, except that 3-aminobiphenyl is replaced with 4-(4-methoxyphenoxy)-aniline (major products MH+=851.6, 867.5).

### Example 43

### Preparation of Compound Mixture 43

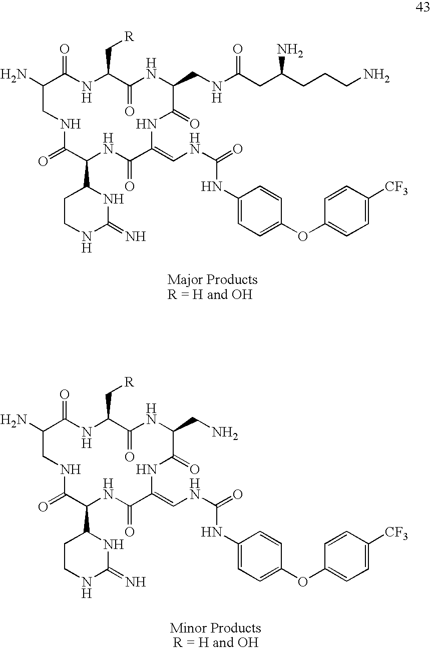

Compound mixture 43 was prepared as an HCl salt according to the procedure outlined in Example 1, except that 3-aminobiphenyl is replaced with 4-(4-trifluoromethoxyphenoxy)-aniline (major products MH+=889.6, 905.5).

### Example 44

### Preparation of Compound Mixture 44

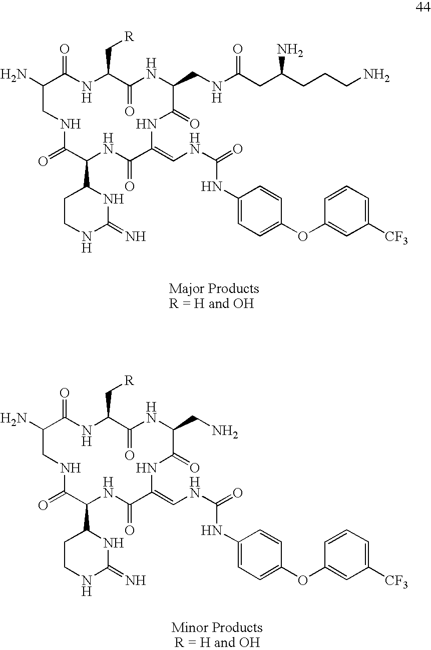

Compound mixture 44 was prepared as an HCl salt according to the procedure outlined in Example 1, except that 3-aminobiphenyl is replaced with 4-(3-trifluoromethoxyphenoxy)-aniline (major products MH+=889.6, 905.4).

### Example 45

### Preparation of Compound Mixture 45

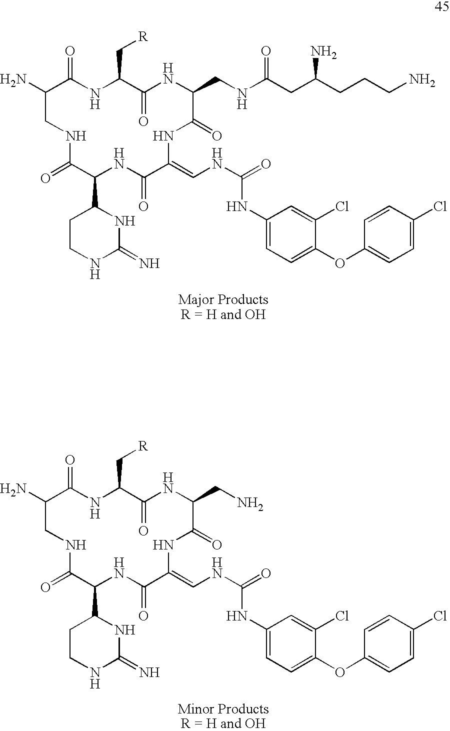

Compound mixture 45 was prepared as an HCl salt according to the procedure outlined in Example 1, except that 3-aminobiphenyl is replaced with 3-chloro-4-(4-chlorophenoxy)-aniline (major products MH+=889.5, 905.4).

### Example 46

### Preparation of Compound Mixture 46

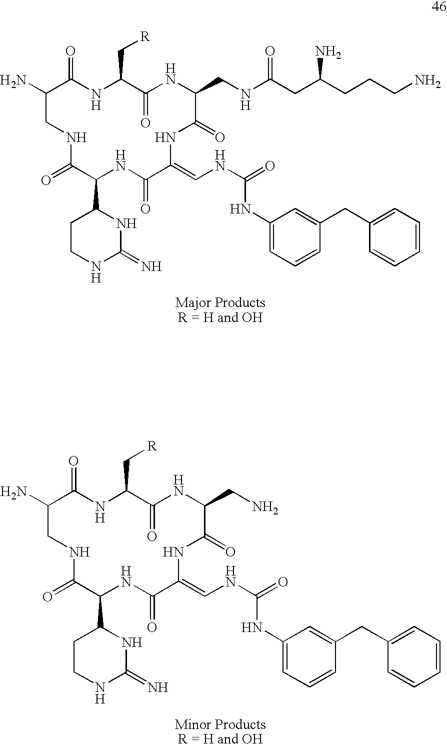

Compound mixture 46 was prepared as an HCl salt according to the procedure outlined in Example 1, except that 3-aminobiphenyl is replaced with 3-benzylaniline (major products MH+=819.6, 835.6).

### Example 47

### Preparation of Compound Mixture 47

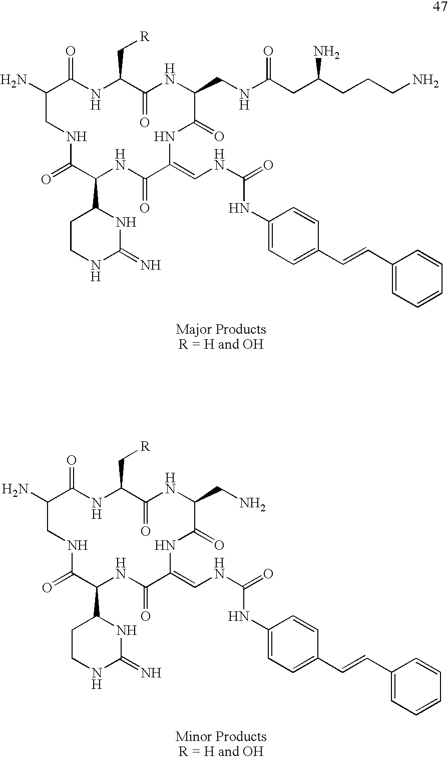

Compound mixture 47 was prepared as an HCl salt according to the procedure outlined in Example 1, except that 3-aminobiphenyl is replaced with (E)-4-styrylbenzenamine (major products MH+=831.6, 847.6).

### Example 48

### Preparation of Compound Mixture 48

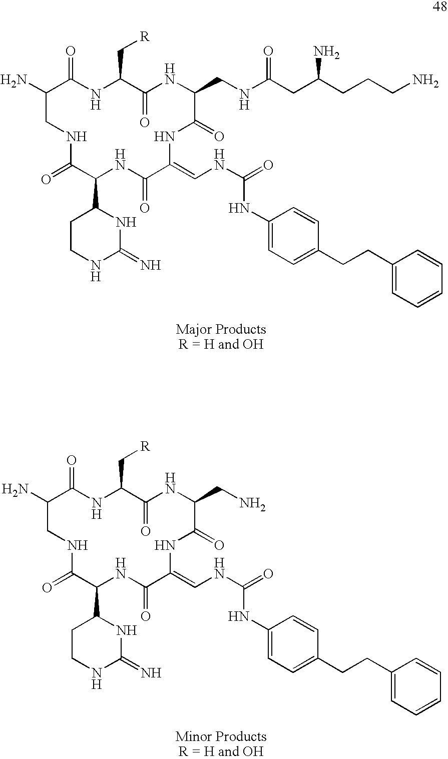

Compound mixture 48 was prepared as an HCl salt according to the procedure outlined in Example 1, except that 3-aminobiphenyl is replaced with 4-phenethylbenzenamine (major products MH+=833.6, 849.6).

### Example 49

### Preparation of Compound Mixture 49

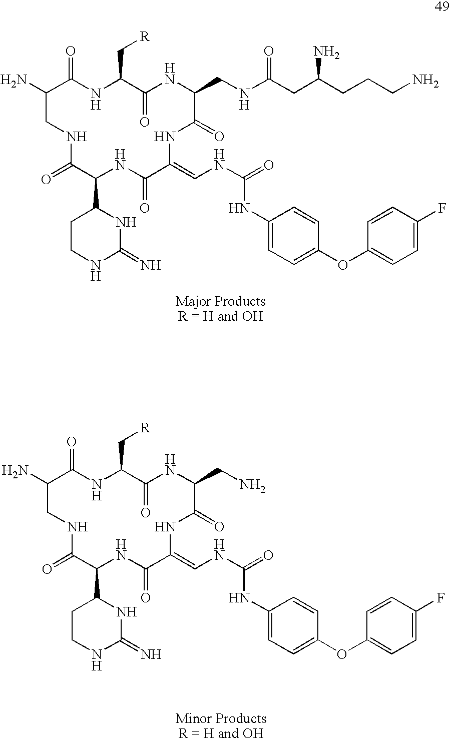

Compound mixture 49 was prepared as an HCl salt according to the procedure outlined in Example 1, except that 3-aminobiphenyl is replaced with 4-(4-fluorophenoxy)-aniline (major products MH+=839.5, 855.5).

### Example 50

### Preparation of Compound Mixture 50

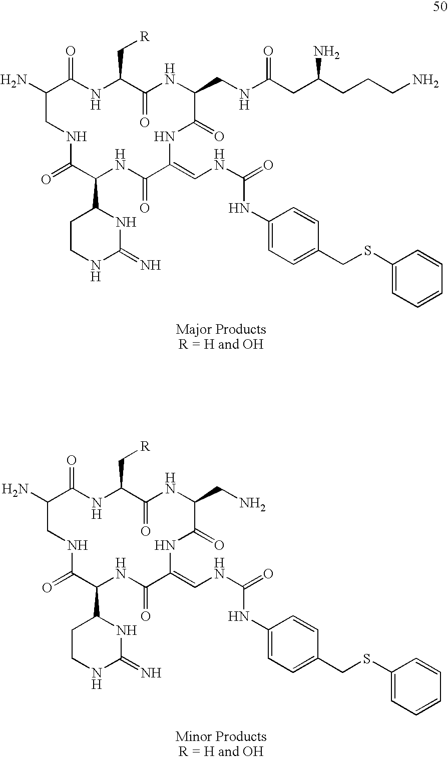

Compound mixture 50 was prepared as an HCl salt according to the procedure outlined in Example 1, except that 3-aminobiphenyl is replaced with 4-(phenylthiomethyl)benzenamine (major products MH+=851.5, 867.4).

### Example 51

### Preparation of Compound Mixture 51

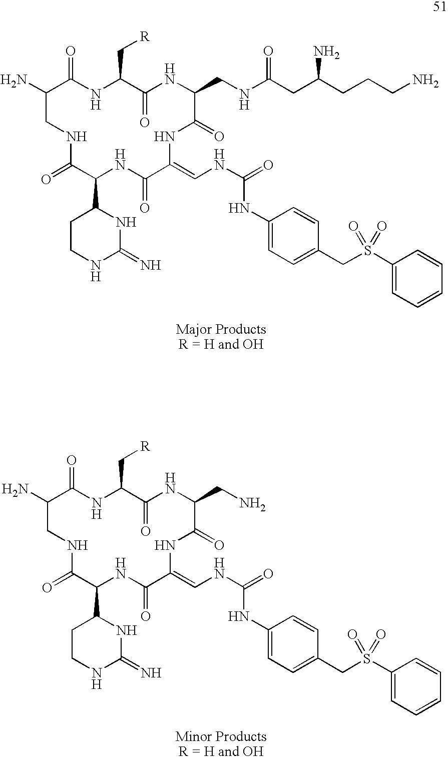

Compound mixture 51 was prepared as an HCl salt according to the procedure outlined in Example 1, except that 3-aminobiphenyl is replaced with 4-(phenylsulfonylmethyl)benzenamine (major products MH+=883.5, 899.5).

### Example 52

### Preparation of Compound Mixture 52

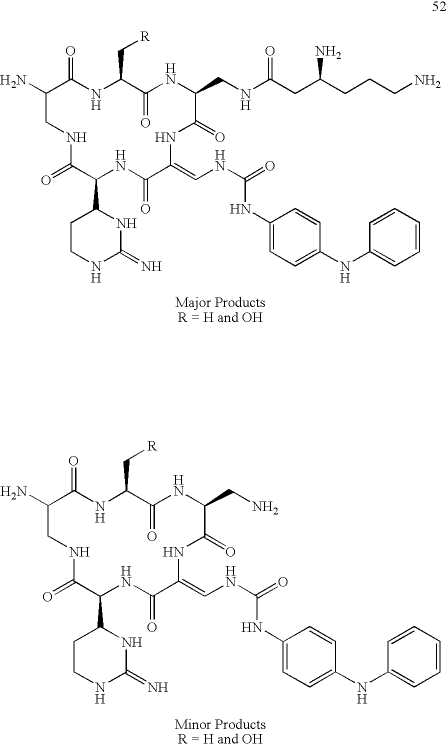

Compound mixture 52 was prepared as an HCl salt according to the procedure outlined in Example 1, except that 3-aminobiphenyl is replaced with N₁-phenylbenzene-1,4-diamine (major products MH+=820.6, 836.6).

### Example 53

### Preparation of Compound Mixture 53

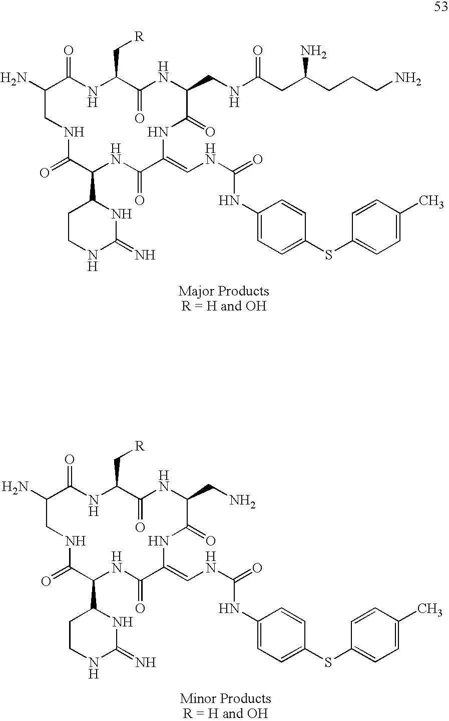

Compound mixture 53 was prepared as an HCl salt according to the procedure outlined in Example 1, except that 3-aminobiphenyl is replaced with 4-(p-tolylthio)benzenamine (major products MH+=851.6, 867.5).

### Example 54

### Preparation of Compound Mixture 54

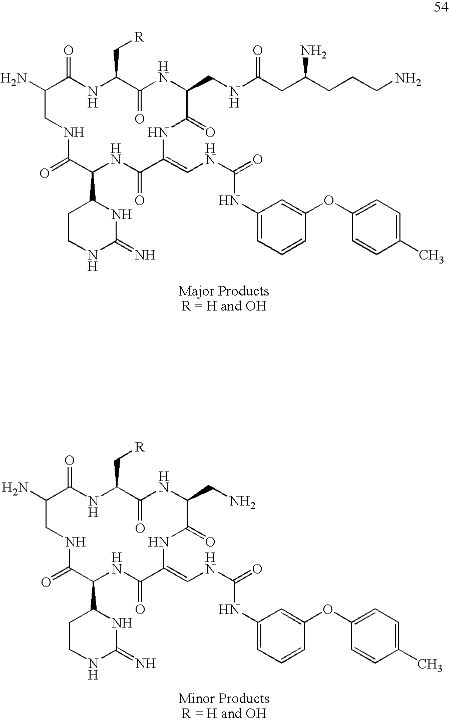

Step A: 1-(3-Hydroxyphenyl)urea (2.00 g), p-tolylboronic acid (3.57 g), and Cu(OAc)₂ (2.63 g) were dissolved in 150 mL of THF, and 5.32 mL of pyridine was added. The reaction was stirred at room temperature overnight, then filtered through Celite with the aid of 200 mL EtOAc. The filtrate was washed with water, 0.2N CuSO₄, 0.5N NaOH, and brine, and dried over Na₂SO₄. The solution was concentrated to a solid that was taken up in 200 mL of dichloromethane and stirred at room temperature for about 1 hour, after which time it was filtered to give 1-(3-(p-tolyloxy)phenyl)urea as white solid. The material was used in Step B without further purification.

Step B: Capreomycin was reacted with 1-(3-(p-tolyloxy)phenyl)urea according to the procedure of Example 1 to give compound mixture 54 as an HCl salt (major products MH+=835.6, 851.5).

### Example 55

### Preparation of Compound Mixture 55

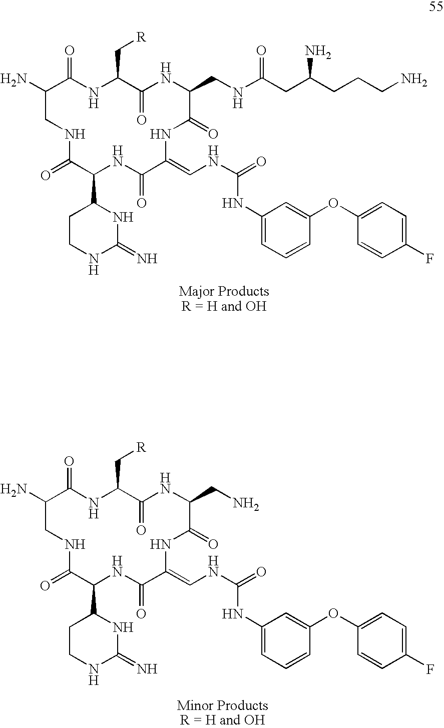

Compound mixture 55 was prepared as an HCl salt according to the procedure of Example 54, except that p-tolylboronic acid was replaced with 4-fluorophenylboronic acid (major products MH+=839.6, 855.6).

### Example 56

### Preparation of Compound Mixture 56

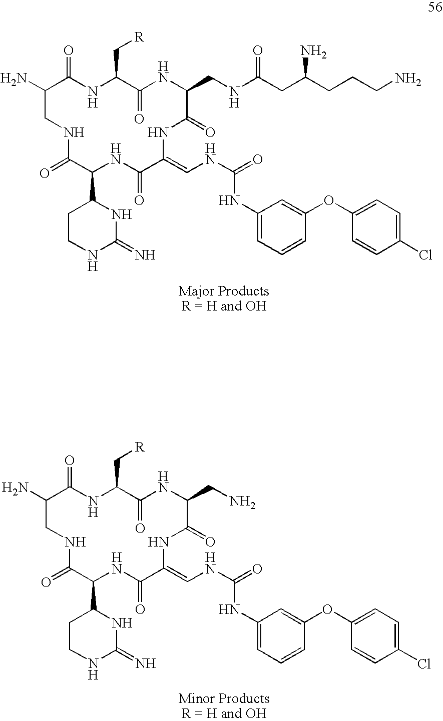

Compound mixture 56 was prepared as an HCl salt according to the procedure of Example 54, except that p-tolylboronic acid was replaced with 4-chlorophenylboronic acid (major products MH+=855.6, 871.5).

### Example 57

### Preparation of Compound Mixture 57

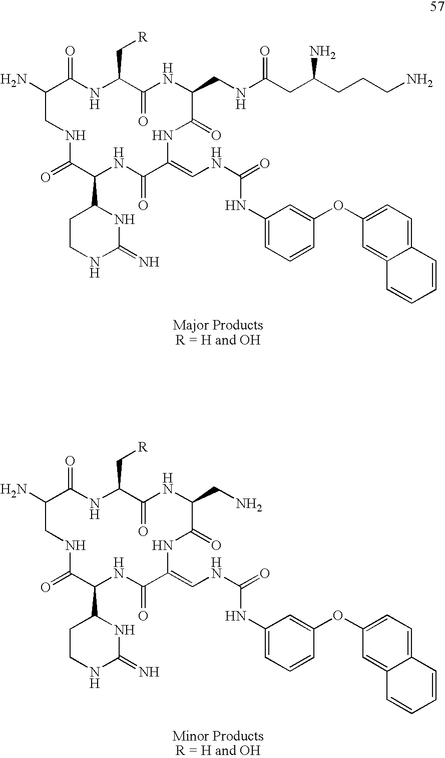

Compound mixture 57 was prepared as an HCl salt according to the procedure of Example 54, except that p-tolylboronic acid was replaced with naphthalen-2-ylboronic acid (major products MH+=871.6, 887.5).

### Example 58

### Preparation of Compound Mixture 58

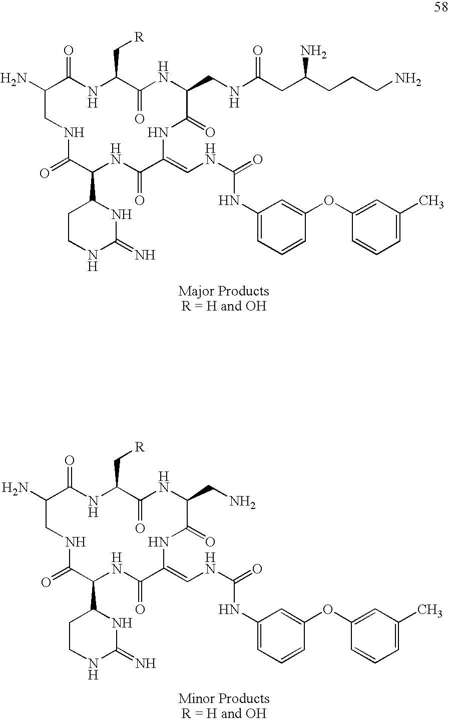

Compound mixture 58 was prepared as an HCl salt according to the procedure of Example 54, except that p-tolylboronic acid was replaced with m-tolylboronic acid (major products MH+=835.6, 851.5).

### Example 59

### Preparation of Compound Mixture 59

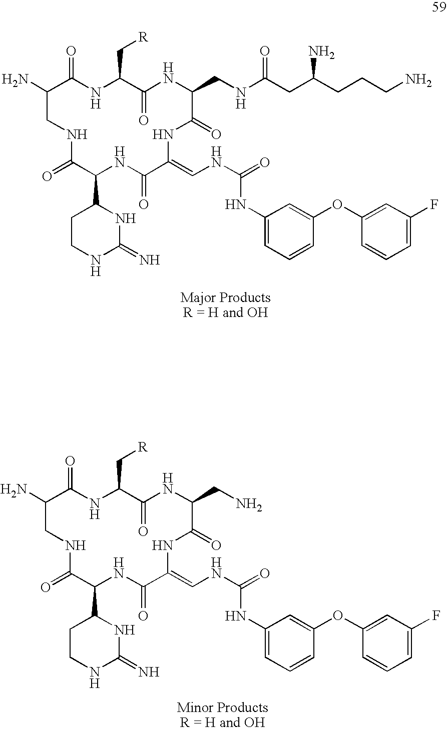

Compound mixture 59 was prepared as an HCl salt according to the procedure of Example 54, except that p-tolylboronic acid was replaced with 3-fluorophenylboronic acid (major products MH+=839.5, 855.6).

### Example 60

### Preparation of Compound Mixture 60

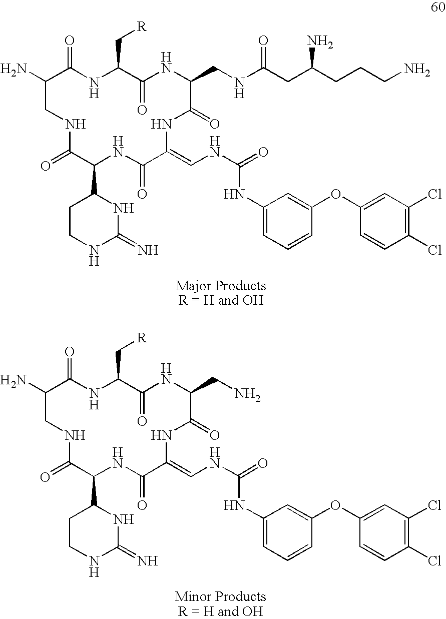

Compound mixture 60 was prepared as an HCl salt according to the procedure of Example 54, except that p-tolylboronic acid was replaced with 3,4-dichlorophenylboronic acid (major products MH+=889.2, 905.3).

### Example 61

### Preparation of Compound Mixture 61

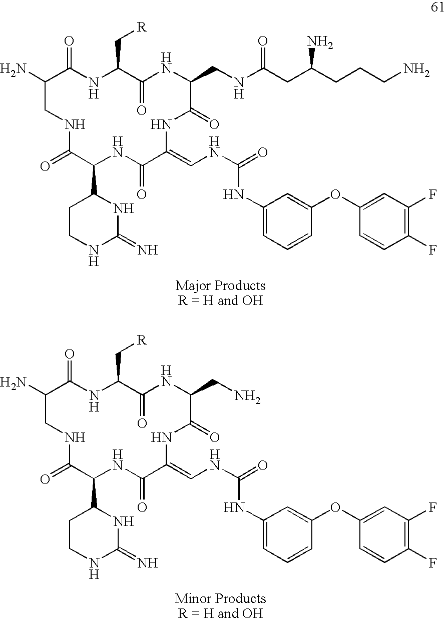

Compound 61 was prepared as an HCl salt according to the procedure of Example 54, except that p-tolylboronic acid was replaced with 3,4-difluorophenylboronic acid (major products MH+=857.3, 873.2).

### Example 62

### Preparation of Compound Mixture 62

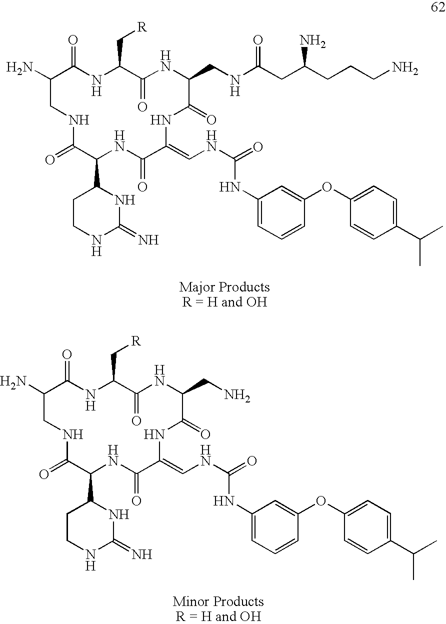

Compound mixture 62 was prepared as an HCl salt according to the procedure of Example 54, except that p-tolylboronic acid was replaced with 4-isopropylphenylboronic acid (major products MH+=863.4, 879.3).

### Example 63

### Preparation of Compound Mixture 63

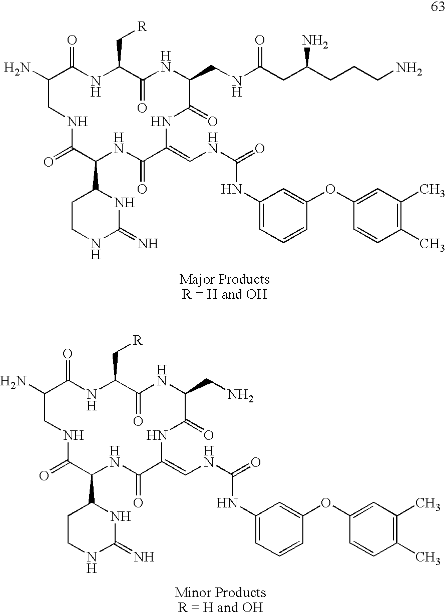

Compound mixture 63 was prepared as an HCl salt according to the procedure of Example 54, except that p-tolylboronic acid was replaced with 3,4-dimethylphenylboronic acid (major products MH+=849.4, 865.3).

### Example 64

### Preparation of Compound Mixture 64

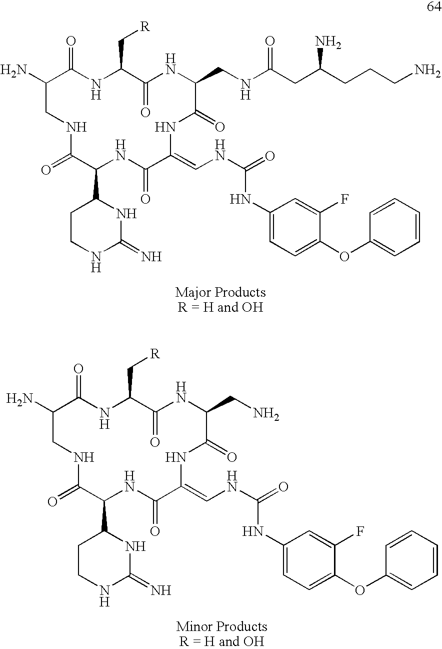

Step A: 1,2-Difluoro-4-nitrobenzene (21.6 g) and phenol (14.0 g) were dissolved in 100 mL of DMF and cooled to 0° C. NaH (6.5 g) was added portionwise and the reaction mixture was stirred at room temperature overnight. The reaction mixture was taken up in EtOAc and washed with water and brine, and dried over Na₂SO₄. The solution was concentrated to provide 2-fluoro-4-nitro-1-phenoxybenzene as an oil, which was used in Step B without further purification.

Step B: 2-Fluoro-4-nitro-1-phenoxybenzene and 7.2 g of 10% Pd/C were dissolved in 200 mL EtOH and stirred under 1 atmosphere of H₂ overnight. The reaction mixture was filtered through Celite with the aid of 50 mL EtOH and concentrated to provide 3-fluoro-4-phenoxybenzenamine as an oil which was used in Step C without further purification.

Step C: Compound mixture 64 was prepared as an HCl salt according to the procedure of Example 1, except that 3-aminobiphenyl was replaced with 3-fluoro-4-phenoxybenzenamine (major products MH+=839.3, 855.3).

### Example 65

### Preparation of Compound Mixture 65

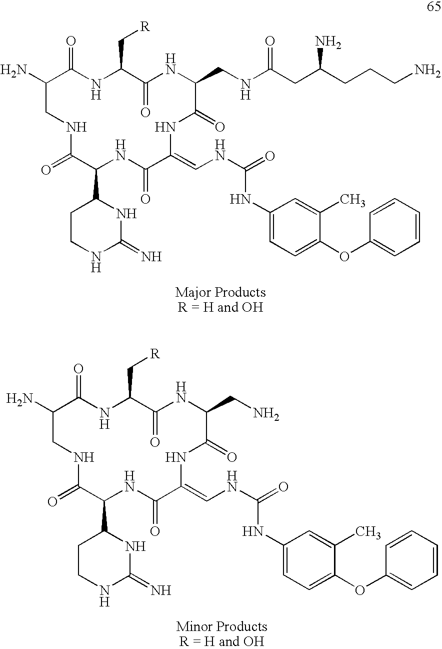

Step A: 1-Fluoro-2-methyl-4-nitrobenzene (9.75 g), phenol (7.10 g), and of Cs₂CO₃ (41.0 g) were dissolved in 100 mL of DMF and heated to 120° C. overnight. The reaction mixture was taken up in EtOAc and washed with water and brine, and dried over Na₂SO₄. The solution was concentrated to provide 2-methyl-4-nitro-1-phenoxybenzene as an oil, which was used in Step B without further purification.

Step B: 2-Methyl-4-nitro-1-phenoxybenzene and 10% Pd/C (3.35 g) were dissolved in 200 mL EtOH and stirred under 1 atmosphere of H₂ overnight. The reaction mixture was filtered through Celite with the aid of 50 mL EtOH and concentrated to provide 3-methyl-4-phenoxybenzenamine as an oil, which was used in Step C without further purification.

Step C: Compound mixture 65 was prepared as an HCl salt according to the procedure of Example 1, except that 3-aminobiphenyl was replaced with 3-methyl-4-phenoxybenzenamine (major products MH+=835.3, 851.3).

### Example 66

### Preparation of Compound Mixture 66

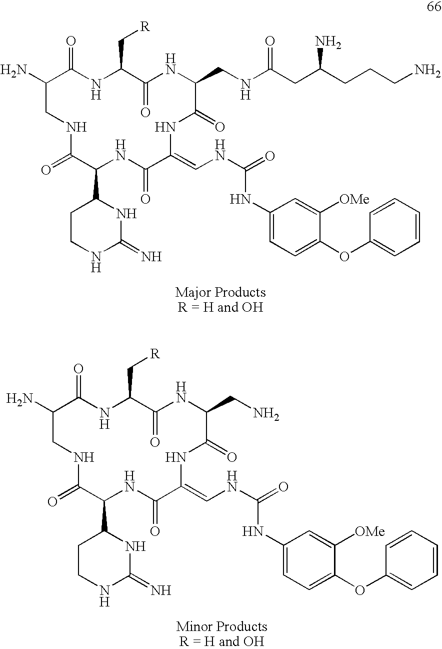

Compound mixture 66 was prepared as an HCl salt according to the procedure of Example 65, except that 1-fluoro-2-methyl-4-nitrobenzene was replaced with 1-chloro-2-methoxy-4-nitrobenzene (major products MH+=851.3, 867.3).

### Example 67

### Preparation of Compound Mixture 67

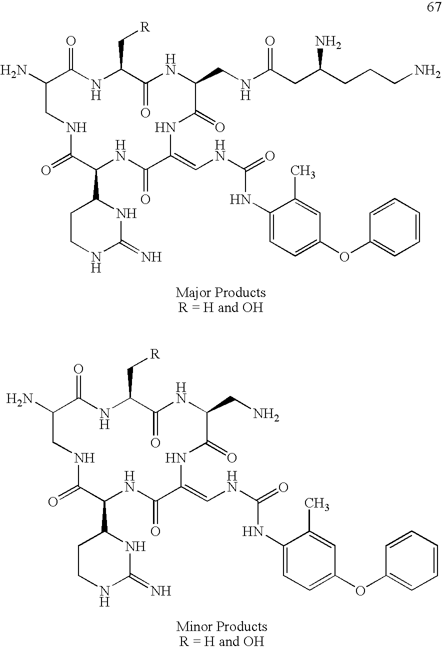

Compound mixture 67 was prepared as an HCl salt according to the procedure of Example 65, except that 1-fluoro-2-methyl-4-nitrobenzene was replaced with 4-fluoro-2-methyl-1-nitrobenzene (major products MH+=835.4, 851.3).

### Example 68

### Preparation of Compound Mixture 68

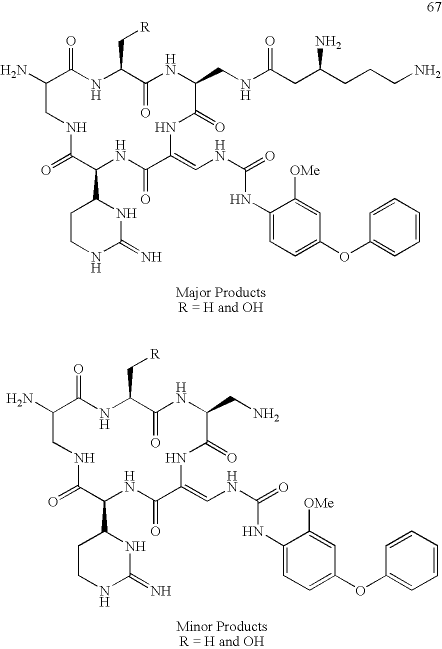

Compound mixture 68 was prepared as an HCl salt according to the procedure of Example 65, except that 1-fluoro-2-methyl-4-nitrobenzene was replaced with 4-chloro-2-methoxy-1-nitrobenzene (major products MH+=853.1, 867.3).

### Example 69

### Preparation of Capreoymcin IIB by Column Chromatography

This example describes a method of preparing a capreomycin derivative of Formula IIB wherein R³, R⁵, R⁶ and R⁷ are H and R⁴ is phenyl.

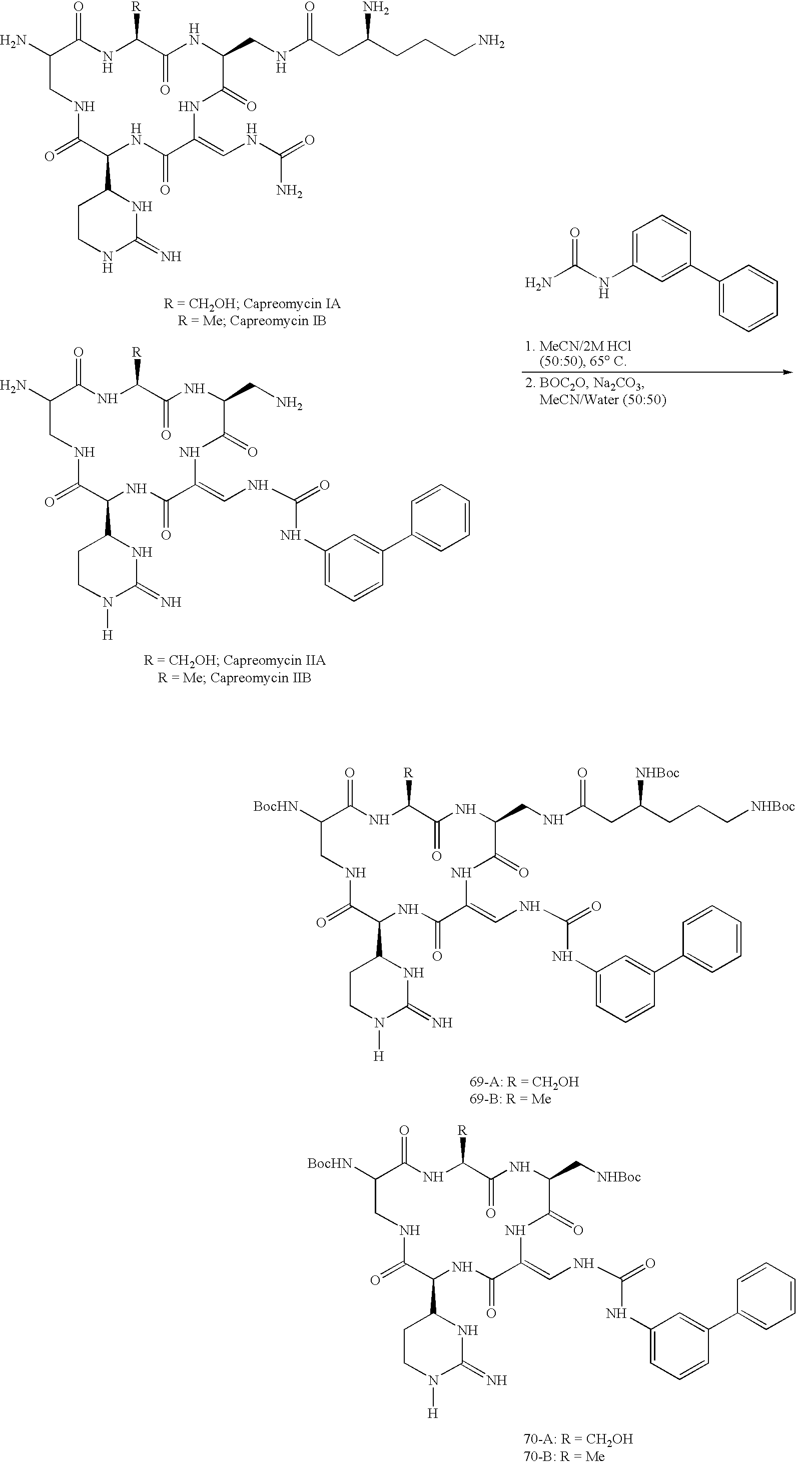

Step A: To a 100 L reaction flask was charged capreomycin sulfate (3.1 kg; capreomycin IB and capreomycin IA 84.8 wt %; capreomycin IIA about 5 wt %, and IIB about 5 wt %), 3-biphenylyl urea (3.5 kg), acetonitrile (26.8 kg, 34.1 L) and 2N HCl (35.77 kg, 34.1 L). The mixture was heated with agitation to 65° C. Agitation was continued until the conversion of capreomycin sulfate to product was ≧90% by HPLC. The reaction mixture was cooled to ambient temperature using an ice/water bath and 25 wt % sodium hydroxide solution (12.7 kg) was charged at a rate to maintain the temperature less than 40° C. providing a pH 7.0 (pH range 6.5-7.5). The slurry mixture was cooled to ambient temperature and agitated for 1 hour. The mixture was then transferred to a 100 L reactor through a tabletop filter funnel by pump rinsing the cake with water (4.7 kg). To the filtrate was charged ethyl acetate (21.6 kg, 24.8 L). The mixture agitated for 10 minutes and allowed to phase split. The top organic layer was transferred to waste. Ethyl acetate (21.6 kg, 24.8 L) was charged to the product rich aqueous layer and the mixture agitated for 10 minutes and allowed to phase split. The top organic layer was transferred to waste, and the bottom layer was used in the next step.

Step B: To the bottom aqueous layer was charged sodium carbonate (1.8 kg), di-tert-butyldicarbonate (2.8 kg) and acetonitrile (38.9 kg, 49.5 L). The mixture was agitated at ambient temperature until the combination of peaks corresponding to compounds 69-A and 69-B were ≧65 area % by HPLC (84.08 area %). To the reaction mixture was charged ethyl acetate (21.7 kg, 24.8 L) and the mixture agitated for 10 minutes and allowed to phase split. The product rich organic layer was transferred to a 100 L cylindrical reactor. The bottom aqueous layer was extracted with ethyl acetate (21.6 kg, 24.8 L). The mixture was agitated for 10 minutes, allowed to phase split, and the bottom aqueous layer was discarded to waste. To the combined organic layers was charged water (24.8 kg). The mixture was agitated for 10 minutes, allowed to phase split and the bottom aqueous layer discarded to waste. To the product rich organic layer was charged water (24.8 kg) and 25 wt % sodium chloride solution (1.2 kg). The mixture was agitated for 10 minutes, allowed to phase split, and the bottom aqueous layer discarded to waste. The product rich organic layer was dried over anhydrous magnesium sulfate (10.8 kg) providing an in-process KF of 3.57 wt %, wherein KF is the Karl Fischer titration value indicating the water content The slurry was filtered and the magnesium sulfate cake washed with ethyl acetate (3.4 kg, 3.9 L). The filtrate was concentrated under vacuum maintaining the bath temperature <41° C. to provide a wet cake which was dried in a vacuum oven at 45° C. until the limit of detection (LOD) was <5 wt % (LOD=3.07 wt %). A product mixture (2.94 kg, 51.3%) containing compounds 69-A, 69-B, 70-A and 70-B was isolated as a pale yellow solid. An HPLC trace of this product mixture is shown in FIG. 1. The peak assignments are as follows: 12.036 minutes: 70-A (4.5 area %); 13.225 minutes: 70-B (4.6 area %); 16.060 minutes: 69-A (42.1 wt %); and 16.813 minutes: 69-B (40.9 wt %).

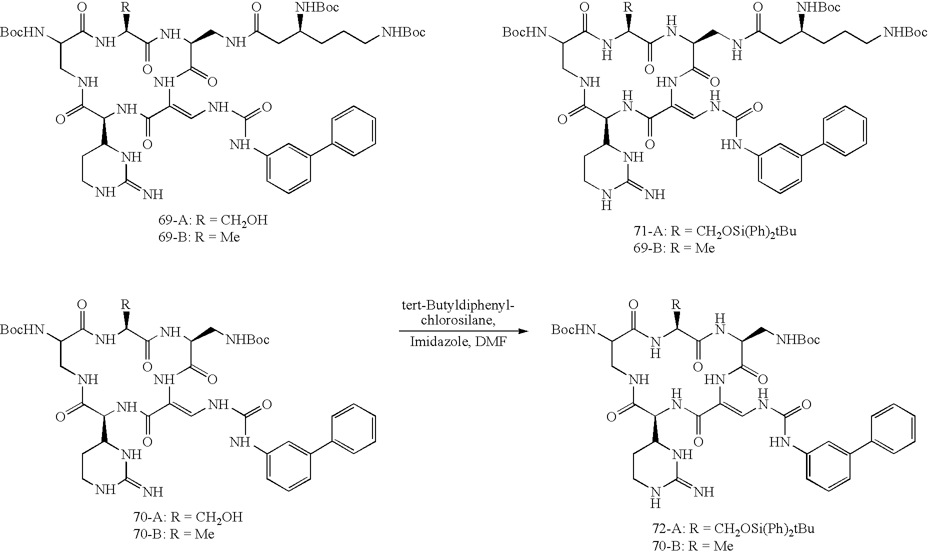

Step C: To a 100 L reaction flask was added 4.4 kg of a product mixture prepared according to Step B (containing 33.42 wt % of compound 69-B), N,N-dimethylformamide (21.2 kg, 22.5 L), imidazole (494 g) and tert-butyldiphenylchlorosilane (1.8 kg). The mixture was agitated at ambient temperature until the ratio of compound 69-B to compound 69-A was ≧97% by HPLC (99.01 area %). The reaction mixture was washed three times with heptane (15.4 kg). Ethyl acetate (20.3 kg, 22.5 L) and water (45 kg) was charged and the mixture agitated for 10 minutes and allowed to phase split. The bottom aqueous layer was extracted with ethyl acetate (20.3 kg) and the combined organic layers were washed twice with a mixture of water (22.5 kg) and 25 wt % sodium chloride solution (3.4 kg). The product rich organic layer was dried over anhydrous magnesium sulfate (3.8 kg), filtered, and the magnesium sulfate cake was washed with ethyl acetate (4.0 kg, 4.4 L). The filtrate was concentrated under vacuum, maintaining the bath temperature <41° C., to provide a wet cake which was dried in a vacuum oven at 45° C. until the LOD was <10 wt % (LOD=5.23 wt %). A product mixture (4.9 kg, 88.8%) containing compounds 71-A, 69-B (27.2 wt %), 72-A and 70-B was isolated as a pale yellow solid.

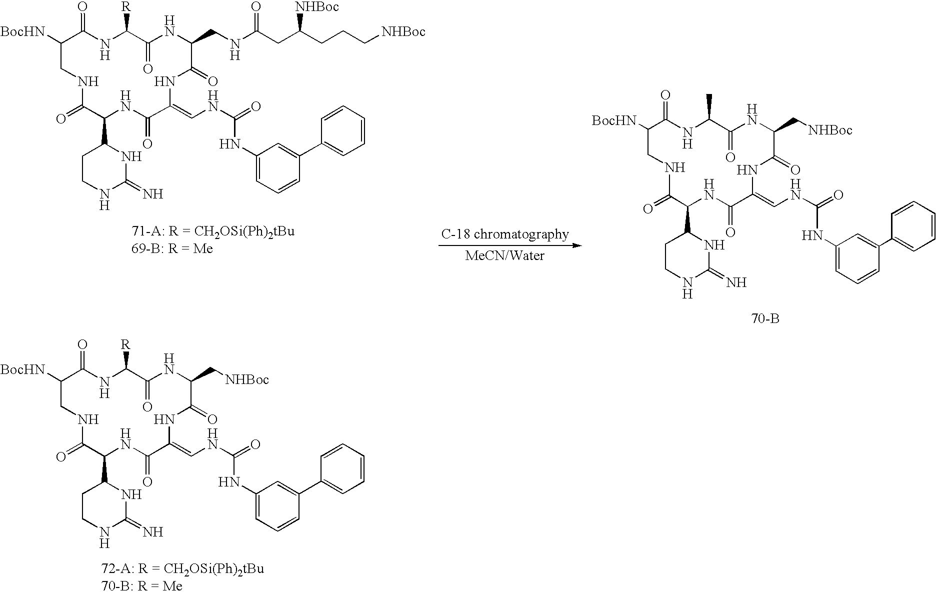

Step D: To a 12 L reaction flask was charged the product mixture containing compounds 71-A, 71-B (27.2 wt %), 72-A and 72-B prepared according to Step C (loading 785-820 g, total 4.8 kg). Water and acetonitrile (2.8-6.6 L, 3.6-8.0 mL/g, 50:50 water:MeCN) were added and the mixture was heated to 40-60° C. Water (707 mL-1.6 L) was charged at a rate so as to maintain the temperature >40° C., providing a final ratio of 60:40 water:acetonitrile. The slurry was then transferred by vacuum to the sample injection module of a Biotage flash 150 chromatography system and charged by pressure onto a C18 reverse phase column in 200-800 mL portions. The column was eluted at a 300 mL/min flow rate using the following solvent system: 15% acetonitrile:water for 5 minutes, then 15 to 50% acetonitrile:water over 90 minutes (linear gradient), followed by 50% acetonitrile:water for 25 minutes, followed by 100% acetonitrile for 25 minutes at 300-400 mL/min. Fractions (500 mL-20 L) were collected manually using a Biotage Flash UV Detector/Recorder Module to detect compound elution. Fractions having >55 area % of compound 72-B were combined in a 100 L cylindrical vessel (75 L). Ethyl acetate (22.5 kg, 25 L) and 20 wt % sodium chloride solution (15.0 L) were charged to the vessel. The mixture was agitated for 10 minutes and then allowed to phase split. The product rich organic layer was concentrated under vacuum maintaining the bath temperature <41° C. to provide a wet cake which was dried in a vacuum oven at 40° C. Compound 70-B was isolated as a colorless solid (46 g, 57 area %).

Step E: The material isolated from Step D was purified on a Biotage flash 60 chromatography system and charged by pressure onto a C18 reverse phase column, eluting with 15% to 20% acetonitrile/water to give 5.2 g of compound 70-B as a white solid.

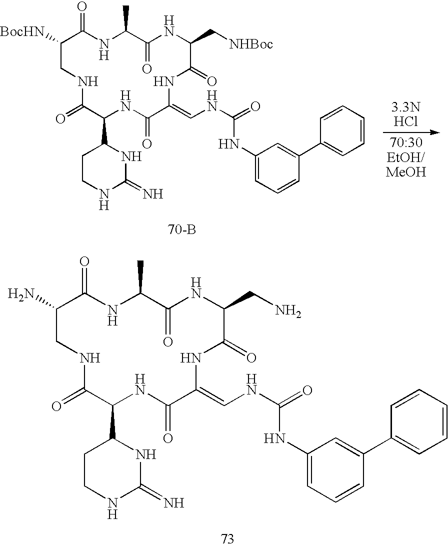

Step F: Compound 70-B (5.2 g) obtained from Step E was dissolved in about 200 mL of 3.3M HCl in 70:30 EtOH/MeOH at room temperature. After stirring for one hour a white solid began to precipitate. After 18 hours the resulting slurry was filtered and the wet cake washed several times with EtOH and dried at room temperature under vacuum to give 3.5 g of compound 73.

### Example 70

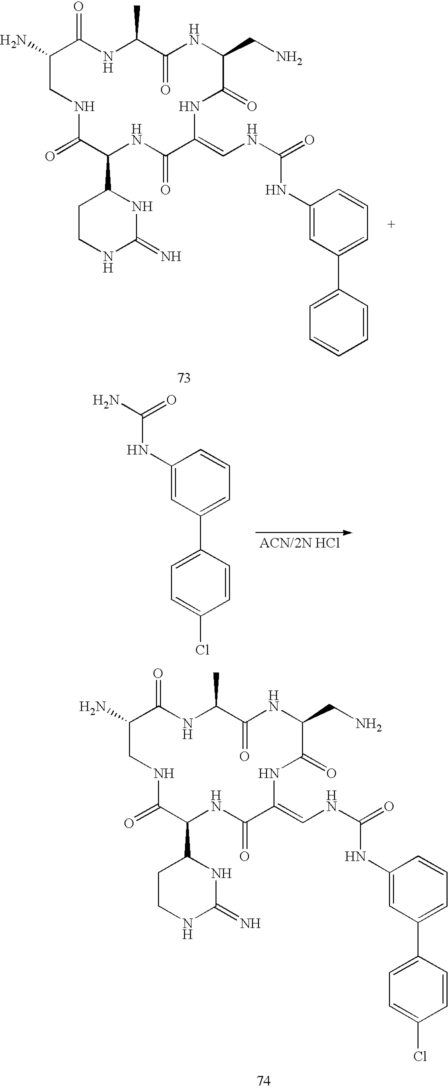

Compound 73 (0.100 g) and 1-(4′-chlorobiphenyl-3-yl)urea (0.365 g) were dissolved/suspended in 8 mL of a 1:1 solution of acetonitrile/2 N aqueous HCl. The reaction mixture was heated at 65° C. overnight. The reaction mixture was cooled to room temperature and filtered. The filtrate was then partitioned between ethyl acetate and water and the aqueous layer was washed once with ethyl acetate. The aqueous layer was concentrated 1-(4′-chlorobiphenyl-3-yl)urea (0.365 g) was added, and the solids were dissolved/suspended in 8 mL of a 1:1 solution of acetonitrile/2 N aqueous HCl. The reaction mixture was heated at 65° C. overnight. The reaction mixture was cooled to room temperature and filtered. The filtrate was then partitioned between ethyl acetate and water and the aqueous layer was washed once with ethyl acetate. The aqueous layer was concentrated to give compound 74 as an HCl salt.

### Example 71

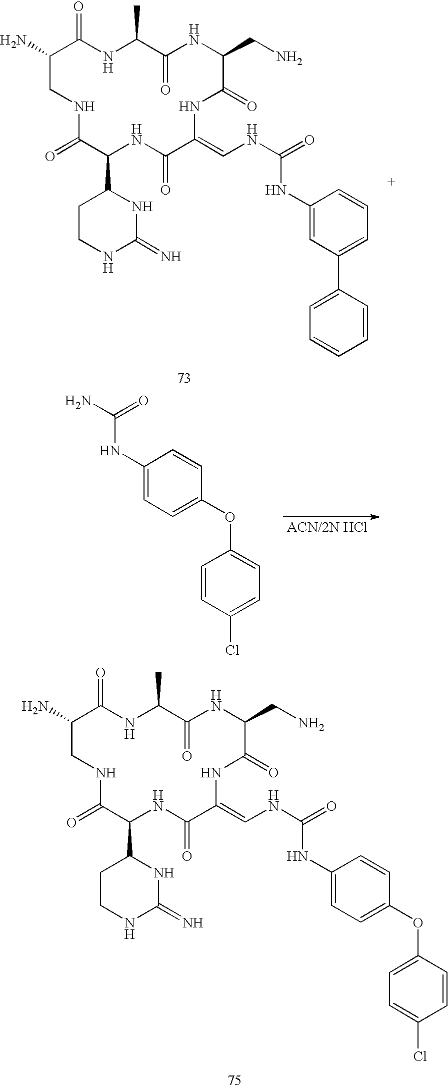

Compound 73 was reacted with 1-(4-(4-chlorophenoxy)phenyl)urea according to the procedure of Example 70 to give compound 75 as an HCl salt.

### Example 72

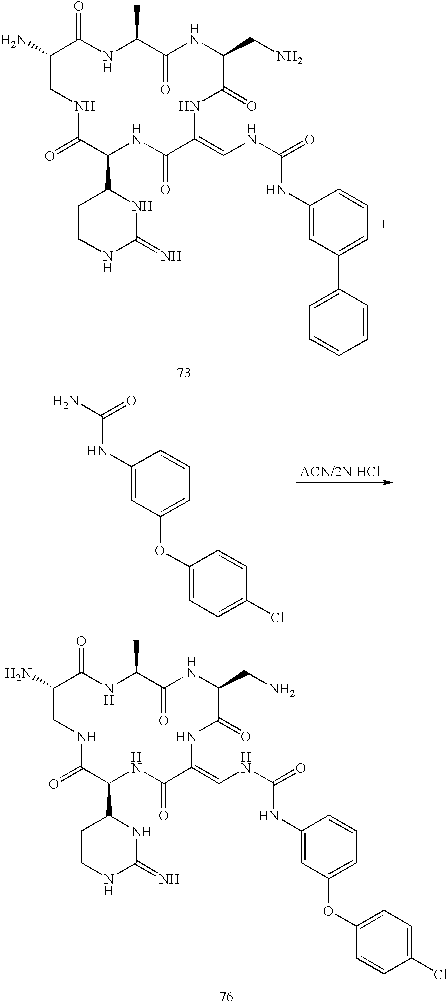

Compound 73 was reacted with 1-(3-(4-chlorophenoxy)phenyl)urea according to the procedure of Example 70 to give compound 76 as an HCl salt.

### Example 73

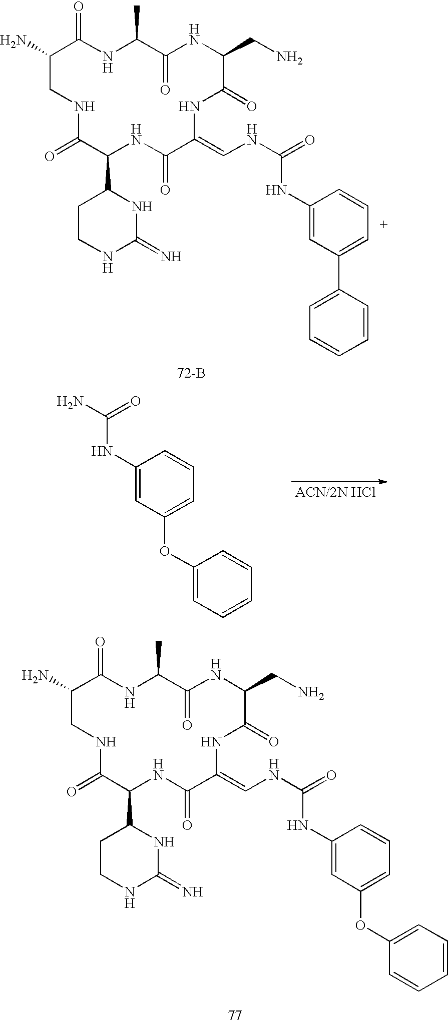

Compound 73 was reacted with 1-(3-phenoxyphenyl)urea according to the procedure of Example 70 to give compound 77 as an HCl salt.

### Example 74

### Production of Capreomycin IIB Via Fermentation of Saccharothrix mutabilis subsp. capreolus

Capreomycin IIB was produced via fermentation with a two-stage seed train. One 2 mL cryovial of Saccharothrix mutabilis subsp. capreolus (AMRI Culture collection MTP 848) was inoculated into 50 mL of a seed medium (30 g/L Nutrisoy 7B Flour (ADM), 20 g/L Cerelose (Corn Products International), 3.3 g/L MgSO₄.7H₂O (Mallinkrodt), 1 g/L CaCO₃ (Mallinkrodt), 0.2 mL/L Antifoam (MAZU) in distilled H₂O and adjusted pH to 7.0). The culture was incubated at 29° C. for 72 hours at 200 rpm with a 1″ throw in a 250 mL flask. After 72 hours of growth, 25 mL from each stage 1 seed flask was aseptically transferred to individual 2.8 L un-baffled Fernbach flasks each containing 600 mL sterile seed medium. These flasks were incubated for 48 hours at 29° C. at 200 rpm with a 1″ throw.

From this second seed stage culture, 0.35 L was then aseptically transferred to a 20 L (TV) fermentor containing 5 g/L (S)-2-aminoethyl-L-cysteine (Sigma) in 12 L of production medium (66 g/L Cerelose (Corn Products International) 3 g/L L-Asparagine (Alfa Aesar) 0.75 g/L CaCl₂.2H₂O (Fluka), 3 g/L MgSO₄.7H₂O (Mallinkrodt), 5 mg/L Fe₂(SO₄)₃ (Sigma), 0.4 g/L KCl (Sigma), 0.2 g/L Na₂HPO₄ (Sigma), 0.2 Ml/l Antifoam (MAZU), and 8 g/L L-Alanine (Sigma) from 100 g/L filter sterilized stock) in distilled H₂O; pH 7.0). The fermentor was initially controlled at 29° C., 0.5 vvm sparged air, back pressure of 10 psi, and 200 rpm agitation. Starting approximately 72 to 90 hours after the initiation of the fermentation, 160 g/L Soytone Select (Difco) in distilled water was fed intermittently as indicated by monitoring of glucose depletion. After 240 hours of fermentation, the entire broth was recovered and ultrafiltered to recover clean fermentation supernatant.

Production of capreomycins was determined by HPLC using an Agilent Zorbax NH₂, (4.6×250 mm 5 micron) column, at ambient temperature 1.5 mL/minute of H₂O (containing 0.5 g/L ammonium bisulfate) and methanol monitored at 268 nm. Capreomycin IIB was produced at 1.1 g/L, representing 76.9% of total capreomycins produced in the fermentation. Total capreomycins were produced at 1.4 g/L with capreomycin IA produced at 0.11 g/L and capreomycin IB produced at 0.17 g/L.

A mixture of capreomycin IIB, IA and IB produced by this method can be reacted with an appropriately substituted phenylurea according to the method of Example 1, Step B to provide a mixture of capreomycin IIB, IA and IB derivatives. The desired capreomycin IIB derivative can then be isolated from this mixture in a manner similar to that described in Example 69.

### Example 75

### Production of Capreomycin IIB via Fermentation of Streptomyces capreolus CAP8-6

Streptomyces capreolus CAP8-6 has been demonstrated to produce capreomycin IIA and IIB selectively when fermented in a particular medium (M. S. Brown, et al., J. Antibiotics, 1997, 696-697). This strain can be fermented in defined media using the procedure of Brown, et al., as follows: agar plugs of cultures are inoculated into 6 mL S-1 medium (1.5% dextrose, 0.5% Difco Bacto Tryptone, 0.25% Difco Yeast Extract, 0.3% MgSO₄.7H₂O, 0.0001% Fe₂(SO₄)₃, and 0.0025% CaCl₂, pH adjusted to 7.0) in 1×6″ culture tubes with metal caps containing 2 glass beads (5 mm), using 6 mm diameter transfer pipets. Tubes are incubated at 29° C., 225 rpm, at an approximately 4° angle. After 48 hours incubation, 0.4 mL of each culture is inoculated into 4 mL of Def-1 medium (6% dextrose, 0.3% L-asparagine, 0.075% CaCl₂2H₂O, 0.3% MgSO₄.7H₂O, 0.0005% Fe₂(SO₄)₃, 0.04% KCl, and 0.02% Na₂HPO₄, pH adjusted to 7.0) in 1×6″ culture tubes with metal caps. L-alanine is optionally added to defined medium as a filter-sterilized, pH 7 stock solution. Tubes are incubated at 29° C., 225 rpm, at an approximately 4° angle for a total of 5 days.

By this method, S. Capreolus CAP8-6 produces capreomycin IIB and IIA in a ratio of 5:1 with 0.8% supplemental L-alanine and in a ratio of 3:1 without supplemental L-alanine.

A mixture of capreomycin IIB and IIA produced by this method can be reacted with an appropriately substituted phenylurea according to the method of Example 1, Step B to provide a mixture of capreomycin IIB and IIA derivatives. The desired capreomycin IIB derivative can then be isolated from this mixture in a manner similar to that described in Example 69.

### Example 76

### Production of Capreomycin IB—Method G-1

This example describes a method of preparing a capreomycin derivative of Formula IB wherein R³, R⁵, R⁶ and R⁷ are H and R⁴ is phenyl.

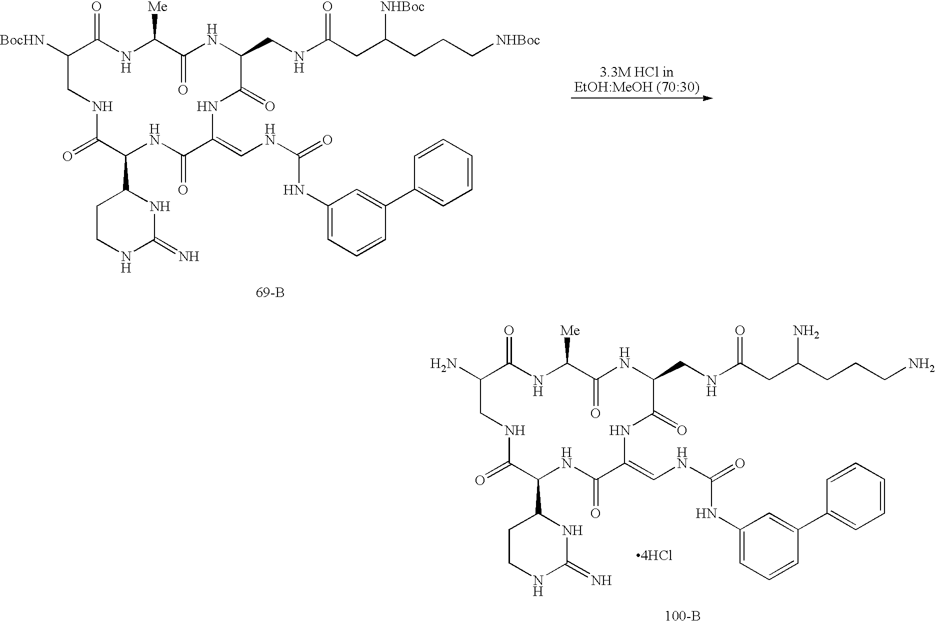

Compound 69-B was prepared according to the method of Example 69, step D, with the exception that the fractions containing compound 69-B were isolated. A 12 L reaction flask was charged with 69-B (347 g, 99.7 wt %). Absolute ethanol 200 proof (2.8 Kg, 3.6 L) was added and the mixture was agitated at ambient temperature until homogeneous. The mixture was transferred by vacuum into a 12 L vessel through a polish filter (Whatman Polycap TF 0.2 micron filter cartridge). Methanol (1.21 Kg, 1.53 L) was added directly to the reaction vessel and the mixture was cooled to a temperature <5° C. HCl gas (626 g) was bubbled into the reaction mixture at a rate so as to maintain the temperature <45° C. The reaction mixture was agitated at ambient temperature until complete as determined by HPLC. The slurry was then filtered and the wet cake washed with absolute ethanol (24.24 Kg, 30.7 L). The wet cake was dried in the vacuum oven at 15-45° C. until the ethanol content was ≦10 wt %. The temperature was then increased to 45-70° C. until the residual ethanol content was ≦5 wt % (preferably ≦1 wt %) to provide the desired compound 100-B (266 g, 97.8 area %). MS (ESI, M+H): for C₃₇H₅₂N₁₄O₇ calculated 805.41, found 805.41. IR (thin film) 1210, 1480, 1490, 1520, 1650, 2900, and 3200 cm⁻¹; ¹H NMR (500 MHz, D₂O) δ 1.33 (3H, d, J 7.3, ²⁸CH₃), 1.62 (1H, m, ²³CH₂), 1.70 (2H, m, ³⁴CH₂), 1.70 (2H, m, ³⁵CH₂), 1.97 (1H, m, ²³CH₂), 2.56 (1H, dd, J 16.2 and 8.7, ³²CH₂), 2.67 (1H, dd, J 16.8 and 4.7, ³²CH₂), 2.94 (2H, m, ³⁶CH₂), 3.24 (2H, m, ²⁴CH₂), 3.25 (1H, m, ²CH₂), 3.55 (1H, penta, J 7.4, ³³CH), 3.61 (1H, dd, J 14.2 and 10.5, ²CH₂), 3.76 (1H, dd, J 14.7 and 10.5, ²CH₂), 4.04 (1H, dd, J 14.0 and 5.1, ²CH₂), 4.22 (1H, dd, J 7.3 and 4.6, ¹CH), 4.26 (1H, m, ²²CH), 4.32 (1H, q, J 7.3, ¹⁴CH), 4.32 (1H, dd, J 10.0 and 5.6, ¹¹CH), 4.84 (1H, dd, J 2.4, ⁵CH), 7.30 (1H, m, ⁴¹CH), 7.32 (1H, m, ⁴³CH), 7.35 (1H, m, ⁴²CH), 7.35 (1H, m, ⁴⁹CH), 7.41 (1H, m, ⁴⁸CH), 7.42 (1H, m, ⁴⁵CH), 7.49 (1H, d, J 7.3, ⁴⁷CH), 8.01 (1H, s, ¹⁷CH). ¹³C NMR (125 MHz, D₂O) δ 18.1, 22.9, 23.1, 29.3, 36.2, 36.6, 38, 39, 39.6, 48.3, 49, 49.2, 51.4, 53.4, 55.1, 105.4, 118.6, 119.5, 123.3, 127.0, 128.1, 129.2, 130, 134.8, 137.6, 139.9, 141.6, 152.1, 154.3, 166.8, 167.1, 171, 172, 172, 175.8. [α]_{D}²⁵=−1.58 (c=20 mg/mL, H₂O, 436 nm).

Isolated 100-B in 95% yield and 90 area %. Observed 5.4-6 area % of an E-isomer. Typical levels of the E-isomer in the wet cake at the conclusion of the reaction were 0.2 to 0.9 area %. Residual EtOH levels were 2.8 to 6 wt %.

### Example 77

### Production of Capreomycin IB—Method G-2

This example describes a method of preparing a capreomycin derivative of Formula IB wherein R³, R⁵, R⁶ and R⁷ are H and R⁴ is phenyl, wherein the level of the E-isomer in the isolated product is less that 1 area %.

In this method, the removal of the Boc-protecting groups was performed on compound 69-B as described in Example 76.

The workup and drying of product 100-B was modified as follows:

Wash volumes 0.1 to 10.0 (preferably 4 to 6). Six Wash Volumes were introduced on the Formulation Pilot Batch reducing the E-isomer level from 5.5 to 0.38 area %.

The isolate wet cake was dried at 40° C. until ethanol content was <10 wt %, and then at 50° C. until ethanol content was ≦1 wt %.

### INDUSTRIAL APPLICABILITY

The compounds of the present subject matter are effective antibacterial agents and useful as medicaments for the treatment of microbial infections and for treating disorders caused by bacterial infections.

The foregoing description is considered as illustrative only of the principles of the present invention. Further, since numerous modifications and changes will be readily apparent to those skilled in the art, it is not desired to limit the present invention to the exact construction and process shown as described above. Accordingly, all suitable modifications and equivalents may be considered to fall within the scope of the present invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. A compound of the Formula IIB 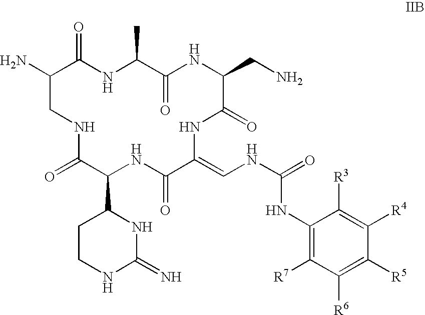
and solvates, metabolites, and pharmaceutically acceptable salts thereof, wherein:
R³, R⁴, R⁵, R⁶ and R⁷ are independently selected from aryl, heteroaryl, X-aryl, X-heteroaryl, hydrogen, halogen, cyano, nitro, trifluoromethyl, difluoromethyl, fluoromethyl, fluoromethoxy, difluoromethoxy, trifluoromethoxy, azido, —OR⁸, SR⁸, —C(O)R⁸, —C(O)OR⁸, NR⁹C(O)OR¹³, —OC(O)R⁸, —NR⁹SO₂R¹³, —SO₂NR⁸R⁹, —NR⁹C(O)R⁸, —C(O)NR⁸R⁹, —NR¹⁰C(O)NR⁸R⁹, —NR¹⁰C(NCN)NR⁸R⁹, —NR⁸R⁹, alkyl, alkenyl, alkynyl, cycloalkyl, cycloalkylalkyl, —S(O)ⱼ(alkyl), —S(O)ⱼ(CR¹¹R¹²)ₘ-aryl, arylalkyl, heteroarylalkyl, heterocyclyl, heterocyclylalkyl, —O(CR¹¹R¹²)ₙ-heterocyclyl or —NR⁹(CR¹¹R¹²)ₙ-heterocyclyl,
wherein at least one of R³, R⁴, R⁵, R⁶ and R⁷ is aryl, heteroaryl, X-aryl or X-heteroaryl, and
wherein said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from oxo, oxime, halogen, cyano, nitro, fluoromethyl, difluoromethyl, trifluoromethyl, fluoromethoxy, difluoromethoxy, trifluoromethoxy, azido, alkyl, alkenyl, alkynyl, cycloalkyl, cycloalkylalkyl, —OR⁸, —C═NOR⁸, —C(O)R⁸, —C(O)OR⁸, —OC(O)R⁸, —C(O)NR⁸R⁹, —NR⁸R⁹, —NR⁹C(O)OR¹³, —NR⁹C(O)R⁸, —NR¹⁰C(O)NR⁸R⁹, —NR¹⁰C(NCN)NR⁸R⁹, —O(CR¹¹R¹²)ₙ-aryl, —NR⁸(CR¹¹R¹²)ₘ-aryl, —O(CR¹¹R¹²)ₙ-heteroaryl, —NR⁹(CR¹¹R¹²)ₘ-heteroaryl, —O(CR¹¹R¹²)ₘ-heterocyclyl, —NR⁹(CR¹¹R¹²)ₙ-heterocyclyl, —S(O)ⱼ(alkyl), —S(O)ⱼ(CR¹¹R¹²)ₘ-aryl, —SO₂NR¹¹R¹², —NR⁹SO₂R¹³, aryl, heteroaryl, arylalkyl, heteroarylalkyl, heterocyclyl and heterocyclylalkyl;
X is O, O(CR¹¹R¹²)ₙ, NR⁹, (CR¹¹R¹²)ₙ, CR¹¹═CR¹², or S(O)ⱼ(CR¹¹R¹²)ₘ;
R⁸ is hydrogen, trifluoromethyl, alkyl, alkenyl, alkynyl, cycloalkyl, cycloalkylalkyl, aryl, arylalkyl, heteroaryl, heteroarylalkyl, heterocyclyl, heterocyclylalkyl, phosphate, or an amino acid residue, wherein any of said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from oxo, halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″,
—SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl;
R⁹, R¹⁰, R¹¹ and R¹² are independently hydrogen or alkyl, and
R¹³ is trifluoromethyl, alkyl, cycloalkyl, aryl, arylalkyl, heteroaryl, heteroarylalkyl, heterocyclyl, or heterocyclylalkyl, wherein any of said alkyl, cycloalkyl, aryl, arylalkyl, heteroaryl, heteroarylalkyl, heterocyclyl and heterocyclylalkyl portions are optionally substituted with one or more groups independently selected from oxo, halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″,
—SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,
or R⁸ and R⁹ together with the atoms to which they are attached form a 4 to 10 membered saturated, partially unsaturated, or fully unsaturated heterocyclic ring, wherein said heterocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,
or R⁹ and R¹⁰ together with the atoms to which they are attached form a 4 to 10 membered saturated, partially unsaturated, or fully unsaturated heterocyclic ring, wherein said heterocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,
or R⁹ and R¹¹ together with the atoms to which they are attached form a 4 to 10 membered saturated, partially unsaturated, or fully unsaturated heterocyclic ring, wherein said heterocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,
or R⁹ and R¹³ together with the atoms to which they are attached form a 4 to 10 membered saturated, partially unsaturated, or fully unsaturated heterocyclic ring, wherein said heterocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,
or R¹¹ and R¹² together with the atoms to which they are attached form a 4 to 10 membered saturated, partially unsaturated, or fully unsaturated carbocyclic ring, wherein said carbocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl;
R′, R″ and R′″ independently are hydrogen, alkyl, alkenyl, aryl and arylalkyl, and R″″ is alkyl, alkenyl, aryl and arylalkyl, or any two of R′, R″, R′″ or R″″ together with the atoms to which they are attached form a 4 to 10 membered carbocyclic, aryl, heteroaryl or heterocyclic ring, wherein said alkyl, alkenyl, and arylalkyl, and said carbocyclic, aryl, heteroaryl and heterocyclic rings are optionally substituted with one or more groups independently selected from halogen, cyano, nitro, trifluoromethyl, difluoromethoxy, trifluoromethoxy, azido, aryl, heteroaryl, arylalkyl, heteroarylalkyl, heterocyclyl, and heterocyclylalkyl;
m is 0, 1, 2, 3, 4 or 5;
n is 1, 2, 3, 4 or 5; and
j is 0, 1 or 2.

2. The compound of claim 1, wherein:
R³, R⁴, R⁵, R⁶ and R⁷ are independently selected from aryl, heteroaryl, X-aryl, X-heteroaryl, hydrogen, halogen, and alkyl,
wherein at least one of R³, R⁴, R⁵, R⁶ and R⁷ is aryl, heteroaryl, X-aryl or X-heteroaryl, and
wherein said aryl, heteroaryl, and alkyl portions are optionally substituted with one or more groups independently selected from halogen, fluoromethyl, difluoromethyl, trifluoromethyl, fluoromethoxy, difluoromethoxy, trifluoromethoxy, alkyl or —OR⁸;
and R⁸ is hydrogen, trifluoromethyl or alkyl.

3. The compound of claim 1, wherein at least one of R³, R⁴, R⁵, R⁶ and R⁷ is aryl, wherein said aryl is optionally substituted with one or more groups independently selected from halogen, alkyl, OR⁸, fluoromethyl, difluoromethyl, trifluoromethyl, fluoromethoxy, difluoromethoxy, and trifluoromethoxy.

4. The compound of claim 1, wherein said aryl is phenyl or naphthyl, wherein said phenyl and naphthyl are optionally substituted with one or more groups independently selected from F, Cl, CF₃, CH₃, OCH₃, and OCF₃.

5. The compound of claim 1, wherein R⁴, R⁵, R⁶ and R⁷ are H and R³ is phenyl or 4-chlorophenyl.

6. The compound of claim 1, wherein R³, R⁵, R⁶ and R⁷ are H, and R⁴ is phenyl, 4-chlorophenyl, 4-trifluoromethylphenyl, 3-trifluoromethylphenyl, 4-tolyl, 4-trifluoromethoxyphenyl, 1-naphthyl or 2-naphthyl.

7. The compound of claim 1, wherein R³, R⁴, R⁶ and R⁷ are H, and R⁵ is phenyl, 4-chlorophenyl, 4-trifluoromethylphenyl, or 3,5-bis-trifluoromethylphenyl.

8. The compound of claim 1, wherein R³ is CH₃, R⁴, R⁵ and R⁷ are H, and R⁶ is phenyl.

9. The compound of claim 1, wherein at least one of R³, R⁴, R⁵, R⁶ and R⁷ is heteroaryl, wherein said heteroaryl is optionally substituted with one or more groups independently selected from halogen and alkyl.

10. The compound of claim 9, wherein R³, R⁵, R⁶ and R⁷ are H, and R⁴ is 2-pyridyl, 3-pyridyl, 4-pyridyl, 5-pyrimidyl, 4-isoquinolyl, 2-thienyl, 2-chlorothien-4-yl, 3-thienyl, 2-chloro-thien-3-yl, 2-benzo[b]thienyl, 3-benzo[b]thienyl, or 3,5-dimethylisoxazol-4-yl.

11. The compound of claim 9, wherein R³, R⁴, R⁶ and R⁷ are H, and R⁵ is 2-pyridyl, 3-pyridyl, 4-pyridyl, or 2-thienyl.

12. The compound of claim 1, wherein at least one of R³, R⁴, R⁵, R⁶ and R⁷ is X-aryl, wherein said aryl is optionally substituted with one or more groups independently selected from halogen, alkyl, fluoromethyl, difluoromethyl, trifluoromethyl or OR⁸.

13. The compound of claim 12, wherein X is selected from O, S, SCH₂, CH₂, CH₂CH₂, CH═CH, CH₂SO₂ or NH.

14. The compound of claim 1, wherein R⁴, R⁵, R⁶ and R⁷ are H and R³ is O-phenyl.

15. The compound of claim 1, wherein R³, R⁵, R⁶ and R⁷ are H and R⁴ is O-phenyl or CH₂-phenyl.

16. The compound of claim 1, wherein R³, R⁴, R⁶ and R⁷ are H and R⁵ is —O-phenyl, —O-(4-chlorophenyl), —O-(4-fluorophenyl), —O-(4-methylphenyl), —O-(4-methoxyphenyl), —O-(4-trifluoromethylphenyl), —O -(3-trifluoromethylphenyl), 4-(3,5-bis-trifluoromethylphenoxy), —OCH₂-phenyl, —S-phenyl, —CH₂-phenyl, —CH₂CH₂-phenyl, —CH═CH-phenyl, —S—CH₂-phenyl, —CH₂SO₂-phenyl, or —NH-phenyl.

17. The compound of claim 1, wherein R³, R⁶ and R⁷ are H, R⁴ is Cl, and R⁵ is O-(4-chlorophenyl).

18. A composition comprising a compound of claim 1.

19. A method of preparing a compound of claim 1 having the formula IIB, said method comprising:
a) providing a mixture of compounds having formulas IA, IB, IIA and IIB 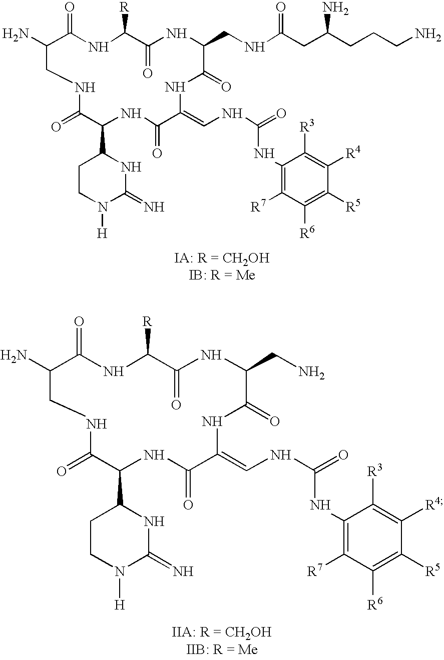
b) treating said mixture from step a) with a reagent that delivers a nitrogen protecting group in the presence of a base and in a suitable organic solvent to provide a mixture of compounds IA-2, IB-2, IIA-2 and IIB-2 having the formulas: 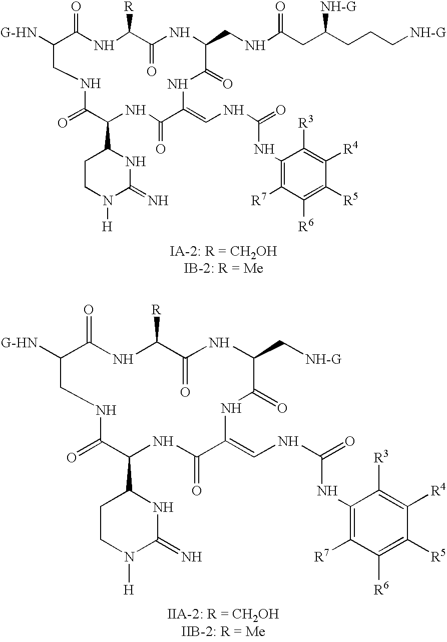
wherein G is a nitrogen protecting group;
c) treating said mixture of compounds IA-2, IB-2, IIA-2 and IIB-2 with a reagent that delivers an alcohol protecting group to provide a mixture of compounds having formulas IA-3, IB-2, IIA-3 and IIB-2 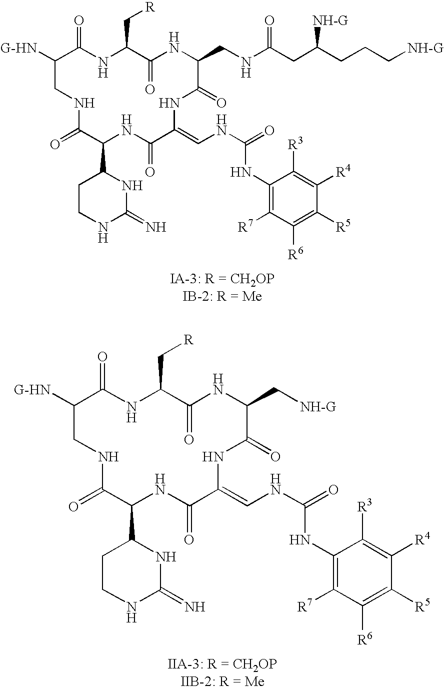
wherein P is an alcohol protecting group;
d) loading the mixture of compounds IA-3, IB-2, IIA-3 and IIB-2 onto a C18 reverse phase resin;
e) eluting fractions containing compound IIB-2 from said resin with a gradient eluent comprising 15-100% acetonitrile:water;
f) collecting said fractions containing compound IIB-2; and
g) removing said nitrogen protecting group from compound IIB-2 to provide said compound having Formula IIB.

20. The method of claim 19, further comprising:
f-1) combining fractions containing compound IIB-2;
f-2) loading the combined fractions onto a second C18 reverse phase column; and
f-3) eluting fractions containing compound IIB-2 from the second column with a gradient eluent comprising 15-100% acetonitrile:water.

21. The method of claim 19, wherein said mixture of compounds having formulas IA, IB, IIA and IIB is prepared by reacting capreomycin sulfate with a reagent having the formula 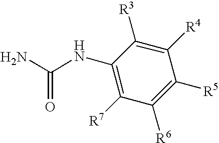
in the presence of an acid.

22. The method of claim 19, wherein said nitrogen protecting group is t-butylcarbonate.

23. The method of claim 19, wherein said alcohol protecting group is tert-butyldiphenylchlorosilane.

24. A method of preparing a compound of claim 1, said method comprising:
a) inoculating Saccarothirix mutaiblis subsp. capreolus into a seed medium at 29° C. for a suitable incubation period;
b) transferring an aliquot of the seed medium from step (a) into a flask containing sterile seed medium;
c) incubating said medium from step (b) at 29° C. for a suitable incubation period;
d) transferring an aliquot of said medium from step (c) into a fermentor containing (S)-2-aminoethyl-L-cysteine in a production medium;
e) adding Soytone Select to said fermentor to provide a mixture;
f) incubating said mixture of step (e) at 29° C.;
g) filtering said production medium to provide a mixture containing capreomycin IIB, capreomycin IA, and capreomycin IB;
h) reacting said mixture containing capreomycin IIB, capreomycin IA, and capreomycin IB with a reagent having the formula 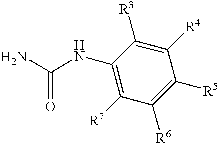
in the presence of an acid, to provide a mixture of compounds having formulas IA, IB, and IB 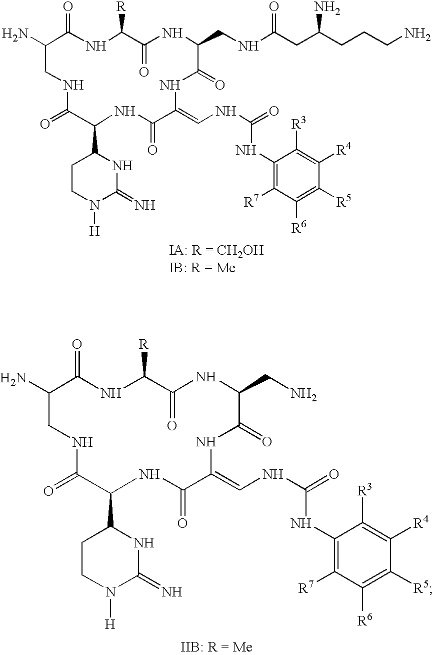
i) treating said mixture from step a) with a reagent that delivers a nitrogen protecting group in the presence of a base and in a suitable organic solvent and to provide a mixture of compounds IA-2, IB-2, and IIB-2 having the formulas: 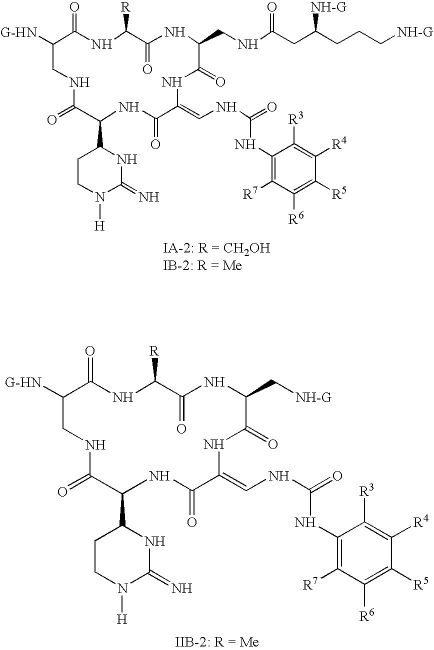
wherein G is a nitrogen protecting group;
j) treating said mixture of IA-2, IB-2, and IIB-2 with a reagent that delivers an alcohol protecting group to provide a mixture of compounds IA-3, IB-2, and IIB-2 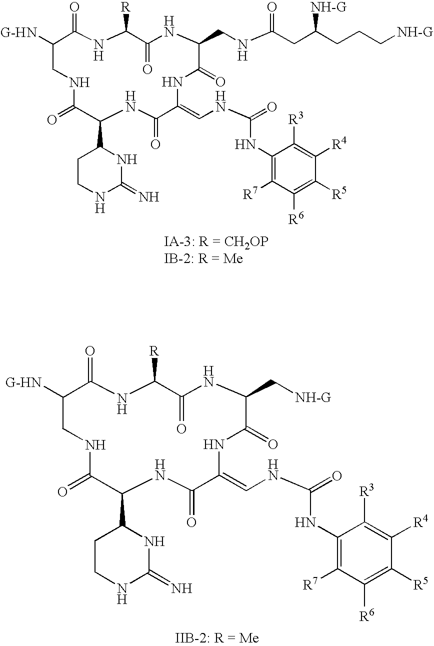
wherein P is an alcohol protecting group;
k) loading the mixture of compounds IA-3, IB-2, and IIB-2 onto a C18 reverse phase resin;
l) eluting fractions containing compound IIB-2 from said resin with a gradient eluent comprising 15-100% acetonitrile:water;
m) collecting said fractions containing compound IIB-2; and
n) removing said nitrogen protecting group from compound IIB-2 to provide said compound having Formula IIB.

25. The method of claim 24, wherein the incubation period in step (a) is 72 hours.

26. The method of claim 24, wherein the incubation period in step (a) is 48 hours.

27. A method of preparing a compound of claim 1, said method comprising:
a) inoculating agar plugs of Streptomyces capreolus CAP8-6 culture in S-1 medium;
b) incubating said culture at 29° C. for a suitable incubation period;
c) inoculated said culture into Def-1 medium;
d) optionally adding 0.8% L-alanine to said culture;
e) incubating either the culture from step c) or d) at 29° C. for a suitable incubation period to provide a mixture containing capreomycin IIA and IIB;
f) treating the mixture of capreomycin IIA and IIB with a reagent having the formula 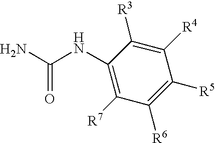
in the presence of an acid, to provide a mixture of compounds having the formulas IIA and IIB 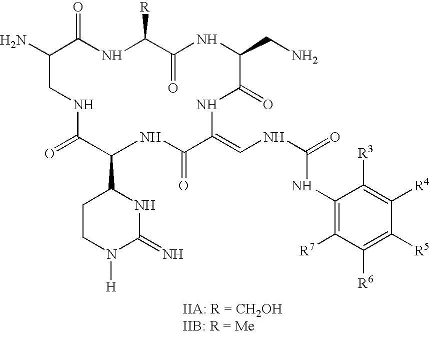
g) treating said mixture from step f) with a reagent that delivers a nitrogen protecting group in the presence of a base and in a suitable organic solvent and to provide a mixture of compounds IIA-2 and IIB-2 having the formulas: 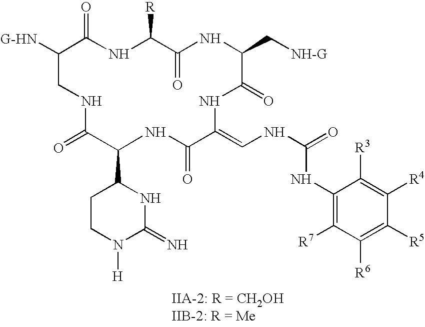
wherein G is a nitrogen protecting group;
h) treating said mixture of compounds IIA-2 and IIB-2 with a reagent that delivers an alcohol protecting group to provide a mixture of compounds having formulas IIA-3 and IIB-2 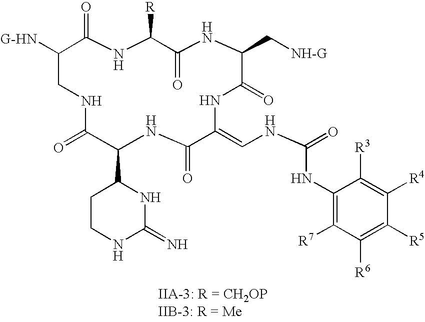
wherein P is an alcohol protecting group;
i) loading the mixture of compounds IIA-3 and IIB-2 onto a C18 reverse phase resin;
j) eluting fractions containing compound IIB-2 from said resin with a gradient eluent comprising 15-100% acetonitrile:water;
k) collecting said fractions containing compound IIB-2; and
l) removing said nitrogen protecting group from compound IIB-2 to provide said compound having Formula IIB.

28. The method of claim 27, wherein the incubation period in step (b) is 48 hours.

29. The method of claim 27, wherein the incubation period in step (e) is 5 days.

30. (canceled)

31. (canceled)

32. (canceled)

33. A method of treating a condition caused by bacterial infection in a mammal, comprising administering to said mammal an effective amount of a compound of claim 1.

34. The method of claim 33, wherein said bacteria is a Gram-negative or Gram-positive pathogen.

35. The method of claim 33, wherein said infection is selected from hospital acquired (nosocomial) infections, pneumonia, otitis media, sinusitis, bronchitis, tonsillitis, mastoiditis, pharyngitis, rheumatic fever, glomerulonephritis, respiratory tract infections, blood and tissue infections, uncomplicated skin and soft tissue infections and abscesses, complicated skin and skin structure infections, puerperal fever, uncomplicated acute urinary tract infections, complicated urinary tract infections, urethritis, cervicitis, sexually transmitted diseases, toxin diseases, ulcers, systemic febrile syndromes, Lyme disease, conjunctivitis, keratitis, dacrocystitis, gastroenteritis, antibiotic-associated diarrhea, colitis, pseudomembraneous colitis, odontogenic infection, persistent cough, gas gangrene, atherosclerosis and cardiovascular disease.

36. The method of claim 33, wherein said bacteria is Clostridium difficile.

37. A method of preparing a compound of claim 1, comprising:
a) reacting a compound having the formula 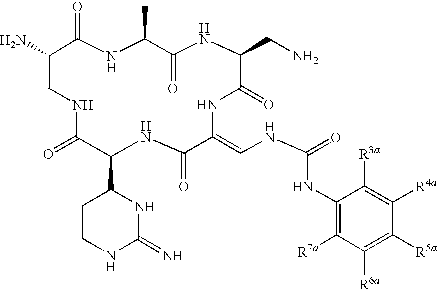
wherein R^{3a}, R^{4a}, R^{5a}, R^{6a} and R^{7a} are as defined for R³, R⁴, R⁵, R⁶ and R⁷, with a substituted phenylurea having the formula 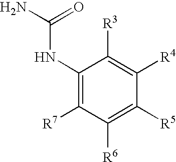
wherein R³, R⁴, R⁵, R⁶ and R⁷ are as defined above, provided that R³ is not the same as R^{3a}, and/or R⁴ is not the same as R^{4a}, and/or R⁵ is not the same as R^{5a}, and/or R⁶ is not the same as R^{6a}, and/or R⁷ is not the same as R^{7a}, in the presence of an acid.

38. A method of preparing a compound of claim 1 having the formula IB 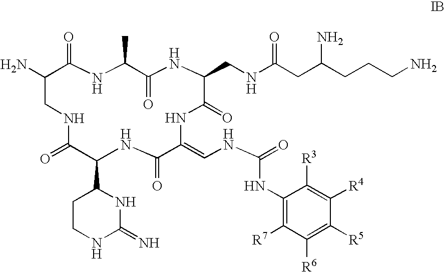
wherein:
R³, R⁴, R⁵, R⁶ and R⁷ are independently selected from aryl, heteroaryl, X-aryl, X-heteroaryl, hydrogen, halogen, cyano, nitro, trifluoromethyl, difluoromethyl, fluoromethyl, fluoromethoxy, difluoromethoxy, trifluoromethoxy, azido, —OR⁸, SR⁸, —C(O)R⁸, —C(O)OR⁸, NR⁹C(O)OR¹³, —OC(O)R⁸, —NR⁹SO₂R¹³, —SO₂NR⁸R⁹, —NR⁹C(O)R⁸, —C(O)NR⁸R⁹, —NR¹⁰C(O)NR⁸R⁹, —NR¹⁰C(NCN)NR⁸R⁹, —NR⁸R⁹, alkyl, alkenyl, alkynyl, cycloalkyl, cycloalkylalkyl, —S(O)ⱼ(alkyl), —S(O)ⱼ(CR¹¹R¹²)ₘ-aryl, arylalkyl, heteroarylalkyl, heterocyclyl, heterocyclylalkyl, —O(CR¹¹R¹²)ₙ-heterocyclyl or —NR⁹(CR¹¹R¹²)ₙ-heterocyclyl,
wherein at least one of R³, R⁴, R⁵, R⁶ and R⁷ is aryl, heteroaryl, X-aryl or X-heteroaryl, and
wherein said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from oxo, oxime, halogen, cyano, nitro, fluoromethyl, difluoromethyl, trifluoromethyl, fluoromethoxy, difluoromethoxy, trifluoromethoxy, azido, alkyl, alkenyl, alkynyl, cycloalkyl, cycloalkylalkyl, —OR⁸, —C═NOR⁸, —C(O)R⁸, —C(O)OR⁸, —OC(O)R⁸, —C(O)NR⁸R⁹, —NR⁸R⁹, —NR⁹C(O)OR¹³, —NR⁹C(O)R⁸, —NR¹⁰C(O)NR⁸R⁹, —NR¹⁰C(NCN)NR⁸R⁹, —O(CR¹¹R¹²)aryl NR⁹(CR¹¹R¹²)ₘ-aryl, —O(CR¹¹R¹²)ₙ-heteroaryl, —NR⁹(CR¹¹R¹²)ₘ-heteroaryl, —O(CR¹¹R¹²)ₙ-heterocyclyl, —NR⁹(CR¹¹R¹²)ₙ-heterocyclyl, —S(O)ⱼ(alkyl), —S(O)ⱼ(CR¹¹R¹²)ₘ-aryl, —SO₂NR⁸R⁹, —NR⁹SO₂R¹³, aryl, heteroaryl, arylalkyl, heteroarylalkyl, heterocyclyl and heterocyclylalkyl,
X is O, O(CR¹¹R¹²)ₙ, NR⁹, (CR¹¹R¹²)ₙ, CR¹¹═CR¹² or S(O)ⱼ(CR¹¹R¹²)ₘ;
R⁸ is hydrogen, trifluoromethyl, alkyl, alkenyl, alkynyl, cycloalkyl, cycloalkylalkyl, aryl, arylalkyl, heteroaryl, heteroarylalkyl, heterocyclyl, heterocyclylalkyl, phosphate, or an amino acid residue, wherein any of said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from oxo, halogen, cyano, nitro, trifluoromethyl, difluoromethoxy, trifluoromethoxy, azido, —NR′SO_R″″, —SO₂NR′R″, —C(O)R″″, —C(O)OR′, —OC(O)R′, —NR′C(O)OR″″, —NR′C(O)R″, —C(O)NR′R″,
—SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,
R⁹, R¹⁰, R¹¹ and R¹² are independently hydrogen or alkyl, and
R¹³ is trifluoromethyl, alkyl, cycloalkyl, aryl, arylalkyl, heteroaryl, heteroarylalkyl, heterocyclyl, or heterocyclylalkyl, wherein any of said alkyl, cycloalkyl, aryl, arylalkyl, heteroaryl, heteroarylalkyl, heterocyclyl and heterocyclylalkyl portions are optionally substituted with one or more groups independently selected from oxo, halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″,
—SO₂R″″, —NR′R″, —NR′C(O)NR′R′″, —NR′C(NCN)NR″R′″, —OR′, aryl, heteroaryl, arylalkyl, heteroarylalkyl, heterocyclyl, and heterocyclylalkyl,
or R⁸ and R⁹ together with the atoms to which they are attached form a 4 to 10 membered saturated, partially unsaturated, or fully unsaturated heterocyclic ring, wherein said heterocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl, or R⁹ and R¹⁰ together with the atoms to which they are attached form a 4 to 10 membered saturated, partially unsaturated, or fully unsaturated heterocyclic ring, wherein said heterocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO_R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,
or R⁹ and R¹¹ together with the atoms to which they are attached form a 4 to 10 membered saturated, partially unsaturated, or fully unsaturated heterocyclic ring, wherein said heterocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,
or R⁹ and R¹³ together with the atoms to which they are attached form a 4 to 10 membered saturated, partially unsaturated, or fully unsaturated heterocyclic ring, wherein said heterocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR′, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl,
or R¹¹ and R¹² together with the atoms to which they are attached form a 4 to 10 membered saturated, partially unsaturated, or fully unsaturated carbocyclic ring, wherein said carbocyclic ring is optionally substituted with one or more groups independently selected from halogen, cyano, nitro, trifluoromethyl, difluoromethoxy, trifluoromethoxy, azido, —NR′SO₂R″″, —SO₂NR′R″, —C(O)R″″, —C(O)OR″, —OC(O)R′, —NR′C(O)OR″″, —NR′C(O)R″, —C(O)NR′R″, —SO₂R″″, —NR′R″, —NR′C(O)NR″R′″, —NR′C(NCN)NR″R′″, —OR′, aryl, heteroaryl, arylalkyl, heteroarylalkyl, heterocyclyl, and heterocyclylalkyl;
R′, R″ and R′″ independently are hydrogen, alkyl, alkenyl, aryl and arylalkyl, and R″″ is alkyl, alkenyl, aryl and arylalkyl, or any two of R′, R″, R′″ or R″″ together with the atoms to which they are attached form a 4 to 10 membered carbocyclic, aryl, heteroaryl or heterocyclic ring, wherein said alkyl, alkenyl, and arylalkyl, and said carbocyclic, aryl, heteroaryl and heterocyclic rings are optionally substituted with one or more groups independently selected from halogen, cyano, nitro, trifluoromethyl, difluoromethoxy, trifluoromethoxy, azido, aryl, heteroaryl, arylalkyl, heteroarylalkyl, heterocyclyl, and heterocyclylalkyl;
m is 0, 1, 2, 3, 4 or 5;
n is 1, 2, 3, 4 or 5; and
j is 0, 1 or 2;
said method comprising:
a) providing a mixture of compounds having formulas IA, IB, IIA and IIB 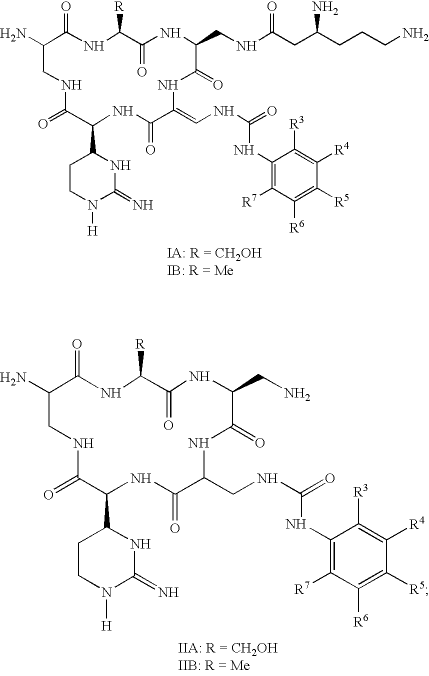
b) treating said mixture from step a) with a reagent that delivers a nitrogen protecting group in the presence of a base and in a suitable organic solvent to provide a mixture of compounds IA-2, IB-2, IIA-2 and IIB-2 having the formulas: 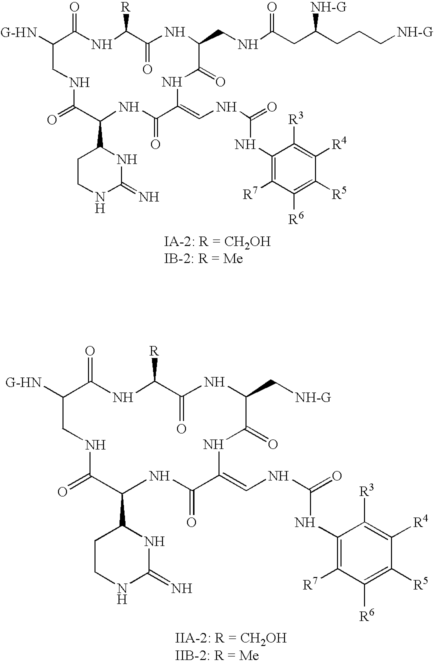
wherein G is a nitrogen protecting group;
c) treating said mixture of compounds IA-2, IB-2, IIA-2 and IIB-2 with a reagent that delivers an alcohol protecting group to provide a mixture of compounds having formulas IA-3, IB-2, IIA-3 and IIB-2 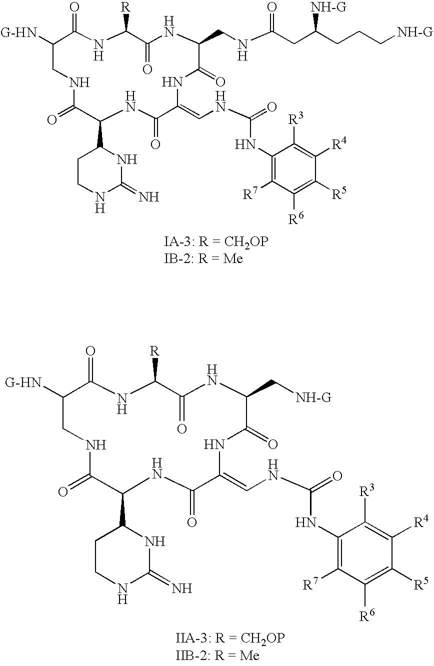
wherein P is an alcohol protecting group;
d) loading the mixture of compounds IA-3, IB-2, IIA-3 and IIB-2 onto a C18 reverse phase resin;
e) eluting fractions containing compound IB-2 from said resin with a gradient eluent comprising 15-100% acetonitrile:water;
f) collecting said fractions containing compound IB-2; and
g) removing said nitrogen protecting group by treating IB-2 with an alcoholic solvent in the presence of HCl to provide said compound having Formula IB.

39. The method of claim 38, further comprising:
h) drying compound IB at a temperature of less than or equal to 40° C. until the alcoholic solvent content is less that 10 wt %; and
i) drying compound IB at a temperature greater than or equal to 45° C. until the alcoholic solvent content is less than or equal to 1 wt %.
